(19) United States
(12) Patent Application Publication
(10) Pub. No.: US 2004/0122026 A1
(43) Pub. Date: Jun. 24, 2004

(54) Imidazolidine compounds

(75) Inventors: Etsuo Ohshima, Nagareyama-shi (JP); Hiroki Sone, Chita-gun (JP); Osamu Kotera, Sunto-gun (JP); Jay R. Luly, Wellesley, MA (US); Gregory J. LaRosa, Newton, MA (US)

Correspondence Address: FITZPATRICK CELLA HARPER & SCINTO, 30 ROCKEFELLER PLAZA, NEW YORK, NY, 10112, US

(73) Assignees: Millennium Pharmaceuticals, Inc., Cambridge, MA; Kyowa Hakko Kogyo Co., Ltd., Chiyoda-ku (JP)

(21) Appl. No.: 10/730,043

(22) Filed: Dec. 9, 2003

Related U.S. Application Data
(62) Division of application No. 10/123,027, filed on Apr. 12, 2002.

(51) Int. Cl. A61K 31/505; A61K 31/415

(52) U.S. Cl. 514/256; 514/400; 544/335; 548/347.1

(57) ABSTRACT

Disclosed are novel compounds and a method of treating inflammatory diseases. The method comprises administering to an individual in need an effective amount of an imidazolidine compound represented by Structural Formula (I):

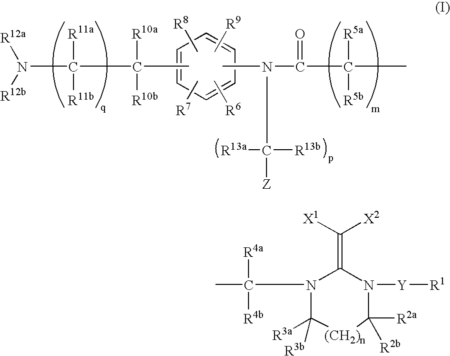

or a physiologically acceptable salt thereof.

### BACKGROUND OF THE INVENTION

Chemokines constitute a family of small cytokines that are produced inflammation and regulate leukocyte recruitment (Baggiolini, M. et al., Adv. Immunol., 55: 97-179 (1994); Springer, T. A., Annu. Rev. Physiol., 57: 827-872 (1995); and Schall, T. J. and K. B. Bacon, Curr. Opin. Immunol., 6: 865-873 (1994)). Chemokines are capable of selectively inducing chemotaxis of the formed elements of the blood (other than red blood cells), including leukocytes such as neutrophils, monocytes, macrophages, eosinophils, basophils, mast cells, and lymphocytes, such as T cells and B cells. In addition to stimulating chemotaxis, other changes can be selectively induced by chemokines in responsive cells, including changes in cell shape, transient rises in the concentration of intracellular free calcium ions ([Ca²⁺]ᵢ), granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

The chemokines are related in primary structure and share four conserved cysteines, which form disulfide bonds. Based upon this conserved cysteine motif, the family can be divided into distinct branches, including the C—X—C chemokines (α-chemokines) in which the first two conserved cysteines are separated by an intervening residue (e.g., IL-8, IP-10, Mig, PF4, ENA-78, GCP-2, GROα, GROβ, GROγ, NAP-2, NAP-4), and the C—C chemokines (β-chemokines), in which the first two conserved cysteines are adjacent residues (e.g., MIP-1α, MIP-1β, RANTES, MCP-1, MCP-2, MCP-3, I-309) (Baggiolini, M. and Dahinden, C. A., Immunology Today, 15:127-133 (1994)). Most CXC-chemokines attract neutrophil leukocytes. For example, the CXC-chemokines interleukin 8 (IL-8), GRO alpha (GROα), and neutrophil-activating peptide 2 (NAP-2) are potent chemoattractants and activators of neutrophils. The CXC-chemokines designated Mig (monokine induced by gamma interferon) and IP-10 (interferon-gamma inducible 10 kDa protein) are particularly active in inducing chemotaxis of activated peripheral blood lymphocytes. CC-chemokines are generally less selective and can attract a variety of leukocyte cell types, including monocytes, eosinophils, basophils, T lymphocytes and natural killer cells. CC-chemokines such as human monocyte chemotactic proteins 1-3 (MCP-1, MCP-2 and MCP-3), RANTES (Regulated on Activation, Normal T Expressed and Secreted), and the macrophage inflammatory proteins 1α and 1β (MIP-1α and MIP-1β) have been characterized as chemoattractants and activators of monocytes or lymphocytes, but do not appear to be chemoattractants for neutrophils.

Chemokines (e.g., CC- and CXC-chemokines) act through receptors which belong to a superfamily of seven transmembrane spanning G protein-coupled receptors (Murphy, P. M., Annu. Rev. Immunol., 12: 593-633 (1994); Gerard, C. and N. P. Gerard, Curr. Opin. Immunol., 6: 140-145 (1994)). This family of G protein-coupled (serpentine) receptors comprises a large group of integral membrane proteins, containing seven transmembrane-spanning regions. The receptors are coupled to G proteins, which are heterotrimeric regulatory proteins capable of binding GTP and mediating signal transduction from coupled receptors, for example, by the production of intracellular mediators.

The chemokine receptors can be divided into groups, which include, CC-chemokine receptors 1 through 9 (CCR1- 9), which can bind certain CC-chemokines, and CXC-chemokine receptors 1 through 4 (CXCR1-4), which can bind certain CXC-chemokines. In general, the CC-chemokine receptors occur on several types of leukocytes, and are important for the migration of monocytes, eosinophils, basophils, and T cells (Qin, S. et al., Eur. J. Immunol., 26: 640-647 (1996); Carr, M. W. et al., Proc. Natl. Acad. Sci. USA, 91(9): 3652-3656 (1994); Taub, D. D. et al., J. Clin. Invest., 95(3): 1370-1376 (1995); Neote, K. et al., Cell, 72: 415-425 (1993); Gao, J.-L. et al., J. Exp. Med., 177: 1421-1427 (1993); Charo, I. F. et al., Proc. Natl. Acad. Sci. USA, 91: 2752-2756 (1994); Myers, S. J. et al., J. Biol. Chem., 270: 5786-5792 (1995); Combadiere, C. et al., J. Biol. Chem., 270(27): 16491-16494 (1995); Ponath, P. D. et al., J. Exp. Med., 183: 2437-2448 (1996); Daugherty, B. L. et al., J. Exp. Med., 183: 2349-2354 (1996); Power, C. A. et al., J. Biol. Chem., 270: 19495-19500 (1995); Hoogewerf, A. J. et al., Biochem. Biophys. Res. Commun., 218: 337-343 (1996); and Samson, M. et al., Biochemistry, 35: 3362-3367 (1996)).

In contrast, the two IL-8 receptors, CXCR1 and CXCR2, are largely restricted to neutrophils and are important for the migration of neutrophils (Baggiolini, M. et al., Adv. Immunol., 55: 97-179 (1994)). The IL-8 receptors, CXCR1 (IL-8R1, interleukin-8 receptor type 1; Holmes, W. E. et al., Science, 253: 1278-1280 (1991)) and CXCR2 (IL-8R2, interleukin-8 receptor type 2; Murphy, P. M. and H. L. Tiffany, Science, 253: 1280-1283 (1991)) both bind IL-8 and appear to recognize the NH₂-terminal Glu-Leu-Arg (ELR) motif as an essential binding epitope observed in CXC-chemokines that induce neutrophil chemotaxis (Clark-Lewis, I. et al., J. Biol. Chem., 266: 23128-23134 (1991); Hert, C. A. et al., J. Biol. Chem., 266: 18989-18994 (1991); and Clark-Lewis, I., et al., Proc. Natl. Acad. Sci. USA, 90: 3574-3577 (1993)). The CXCR1 receptor of human neutrophils binds only IL-8 with high affinity, while the CXCR2 receptor binds IL-8 with similar affinity as CXCR1 but also binds other ELR-containing CXC-chemokines (Baggiolini, M. et al., Adv. Immunol., 55: 97-179 (1994)). Both receptors are capable of coupling to the same G protein a-subunits, exhibiting functional coupling to Gαi2, Gαi 3, Gα14, Gα15, and Gα16 (Wu, et al., Science, 261: 101-103 (1993)). Whether these two receptor subtypes play distinct physiologic roles is not clear.

In contrast to granulocytes and monocytes, lymphocyte responses to chemokines are not well understood. Notably, none of the receptors of known specificity appear to be restricted to lymphocytes and the chemokines that recognize these receptors cannot, therefore, account for events such as the selective recruitment of T lymphocytes that is observed in T cell-mediated inflammatory conditions. Moreover, although a number of proteins with significant sequence similarity and similar tissue and leukocyte subpopulation distribution to known chemokine receptors have been identified and cloned, the ligands for these receptors remain undefined. Thus, these proteins are referred to as orphan receptors. The characterization of the ligand(s) of a receptor, is essential to an understanding of the interaction of chemokines with their target cells, the events stimulated by this interaction, including chemotaxis and cellular activation of leukocytes, and the development of therapies based upon modulation of receptor function.

A chemokine receptor that binds the CXC-chemokines IP-10 and Mig has been cloned and characterized (Loetscher, M. et al., J. Exp. Med., 184: 963-969 (1996)). The receptor mediates Ca²⁺ (calcium ion) mobilization and chemotaxis in response to IP-10 and Mig. CXCR3 expressing cells show no significant response to the CXC-chemokines IL-8, GROα, NAP-2, GCP-2 (granulocyte chemotactic protein-2), ENA78 (epithelial-derived neutrophil-activating peptide 78), PF4 (platelet factor 4), or the CC-chemokines MCP-1, MCP-2, MCP-3, MCP-4, MIP-1α MIP-1β, RANTES, I309, eotaxin or lymphotactin. Moreover, recently a third ligand for CXCR3, I-TAC (Interferon-inducible T cell Alpha Chemoattractant), has also been found to bind to the receptor with high affinity and mediate functional responses (Cole, K. E. et al., J. Exp. Med., 187: 2009-2021 (1998).

The restricted expression of human CXCR3 in activated T lymphocytes and the ligand selectivity of CXCR3 are noteworthy. The human receptor is highly expressed in IL-2 activated T lymphocytes, but was not detected in resting T lymphocytes, monocytes or granulocytes (Qin, S. et al., J. Clin. Invest., 101: 746-754 (1998)). Additional studies of receptor distribution indicate that it is mostly CD3⁺ cells that express CXCR3, including cells which are CD95⁺, CD45RO⁺, and CD45RA^{low}, a phenotype consistent with previous activation, although a proportion of CD20⁺ (B) cells and CD56⁺ (NK) cells also express this receptor. The selective expression in activated T lymphocytes is of interest, because other receptors for chemokines which have been reported to attract lymphocytes (e.g., MCP-1, MCP-2, MCP-3, MIP-1α, MIP-1β, and RANTES) are also expressed by granulocytes, such as neutrophils, eosinophils, and basophils, as well as monocytes. These results suggest that the CXCR3 receptor is involved in the selective recruitment of effector T cells.

CXCR3 recognizes unusual CXC-chemokines, designated IP-10, Mig, and I-TAC. Although these belong to the CXC-subfamily, in contrast to IL-8 and other CXC-chemokines which are potent chemoattractants for neutrophils, the primary targets of IP-10, Mig, and I-TAC are lymphocytes, particularly effector cells such as activated or stimulated T lymphocytes and natural killer (NK) cells (Taub, D. D. et al., J. Exp. Med., 177: 18090-1814 (1993); Taub, D. D. et al., J. Immunol., 155: 3877-3888 (1995); Cole, K. E. et al., J. Exp. Med., 187: 2009-2021 (1998)). (NK cells are large granular lymphocytes, which lack a specific T cell receptor for antigen recognition, but possess cytolytic activity against cells such as tumor cells and virally infected cells.) Consistently, IP-10, Mig, and I-TAC lack the ELR motif, an essential binding epitope in those CXC-chemokines that efficiently induce neutrophil chemotaxis (Clark-Lewis, I. et al., J. Biol. Chem., 266: 23128-23134(1991); Hert, C. A. et al., J. Biol. Chem., 266: 18989-18994 (1991); and Clark-Lewis, I. et al., Proc. Natl. Acad. Sci. USA, 90: 3574-3577 (1993)). In addition, both recombinant human Mig and recombinant human IP-10 have been reported to induce calcium flux in tumor infiltrating lymphocytes (TIL) (Liao, F. et al., J. Exp. Med., 182: 1301-1314 (1995)). While IP-10 has been reported to induce chemotaxis of monocytes in vitro (Taub, D. D. et al., J. Exp. Med., 177: 1809-1814 (1993), the receptor responsible has not been identified), human Mig and I-TAC appear highly selective, and do not show such an effect (Liao, F. et al., J. Exp. Med., 182: 1301-1314 (1995); Cole, K. E. et al., J. Exp. Med., 187: 2009-2021 (1998)). IP-10 expression is induced in a variety of tissues in inflammatory conditions such as psoriasis, fixed drug eruptions, cutaneous delayed-type hypersensitivity responses, tuberculoid leprosy, and in experimental glomerulonephritis, and experimental allergic encephalomyelitis. IP-10 has a potent in vivo antitumor effect that is T cell dependent, is reported to be an inhibitor of angiogenesis in vivo, and can induce chemotaxis and degranulation of NK cells in vitro, suggesting a role as a mediator of NK cell recruitment and degranulation (in tumor cell destruction, for example) (Luster, A. D. and P. Leder, J. Exp. Med., 178: 1057-1065 (1993); Luster, A. D. et al., J. Exp. Med., 182: 219-231 (1995); Angiolillo, A. L. et al., J. Exp. Med., 182: 155-162 (1995); Taub, D. D. et al., J. Immunol., 155: 3877-3888 (1995)). The expression patterns of IP-10, Mig, and I-TAC are also distinct from that of other CXC chemokines in that expression of each is induced by interferon-gamma (IFNγ), while the expression of IL-8 is down-regulated by IFNγ (Luster, A. D. et al., Nature, 315: 672-676 (1985); Farber, J. M., Proc. Natl. Acad. Sci. USA, 87: 5238-5242 (1990); Farber, J. M., Biochem. Biophys. Res. Commun., 192 (1): 223-230 (1993), Liao, F. et al., J. Exp. Med., 182: 1301-1314 (1995); Seitz, M., et al., J. Clin. Invest., 87: 463469 (1991); Galy, A. H. M. and H. Spits, J. Immunol., 147: 3823-3830 (1991); Cole, K. E. et al., J. Exp. Med., 187: 2009-2021 (1998)).

Chemokines are recognized as the long-sought mediators for the recruitment of lymphocytes. Several CC-chemokines were found to elicit lymphocyte chemotaxis (Loetscher, P. et al., FASEB J., 8: 1055-1060 (1994)), however, they are also active on granulocytes and monocytes (Uguccioni, M. et al., Eur. J. Immunol., 25: 64-68 (1995); Baggiolini, M. and C. A. Dahinden, Immunol. Today, 15:127-133 (1994)). The situation is different for IP-10, Mig, and I-TAC, which are selective in their action on lymphocytes, including activated T lymphocytes and NK cells, and which bind CXCR3, a receptor which does not recognize numerous other chemokines and which displays a selective pattern of expression.

In view of these observations, it is reasonable to conclude that the formation of the characteristic infiltrates in inflammatory lesions, such as delayed-type hypersensitivity lesions, sites of viral infection, and certain tumors is a process mediated via CXCR3 and regulated by CXCR3 expression. Lymphocytes, particularly T lymphocytes, bearing a CXCR3 receptor as a result of activation can be recruited into inflammatory lesions, sites of infection, and/or tumors by IP-10, Mig, and/or I-TAC, which can be induced locally by interferon-gamma. Thus, CXCR3 plays a role in the selective recruitment of lymphocytes, particularly effector cells such as activated or stimulated T lymphocytes.

Many existing drugs have been developed as antagonists of the receptors for biogenic amines, for example, as antagonists of the dopamine and histamine receptors. However, no antagonists of the receptors for larger proteins such as chemokines and C5a have been successfully developed and marketed. Small molecule antagonists of the interaction between CXC-chemokine receptors and their ligands, including IP-10, Mig, and I-TAC, would provide compounds useful for inhibiting harmful inflammatory processes “triggered” by receptor ligand interaction, as well as valuable tools for the investigation of receptor-ligand interactions.

Diaminoethylene derivatives possessing an electron withdrawing group(s) are known as a histamine H2 receptor antagonist and a drug useful to treat peptic ulcer (Principles of Medicinal Chemistry, Foye, W. O., Ed. Lea & Febiger, Philadelphia, 1989, 3rd ed.).

### SUMMARY OF THE INVENTION

The present invention relates to small organic compounds which modulate chemokine receptor activity and are useful in the treatment (e.g., palliative therapy, curative therapy, maintenance therapy, prophylactic therapy) of certain diseases or conditions, e.g., inflammatory diseases (e.g., psoriasis), autoimmune diseases (e.g., rheumatoid arthritis, multiple sclerosis), graft rejection (e.g., allograft rejection, xenograft rejection), infectious diseases, cancers. It has now been found that a number of small organic molecules are antagonists of chemokine receptor function (e.g., CXCR3), and can inhibit leukocyte activation and/or recruitment. An antagonist of chemokine receptor function is a molecule which can inhibit the binding of one or more chemokines, such as, CXC-chemokines, for example, IP-10, Mig, and I-TAC, to one or more chemokine receptors on leukocytes and/or other cell types. As a consequence, and by virtue of the fact that antagonists lack chemokine agonist properties, processes and cellular responses mediated by chemokine receptors can be inhibited with these small organic molecules. In one aspect, the invention relates to small organic compounds which are antagonists of CXCR3. Such CXCR3 antagonists can inhibit binding of one or more chemokines (e.g., CXC-chemokines, such as IP-10, Mig and/or I-TAC) to CXCR3.

The invention also relates to a method of modulating (inhibiting or promoting) an inflammatory response in an individual in need of such therapy. The method comprises administering a therapeutically effective amount of a compound (e.g., small organic molecule) which inhibits or promotes mammalian CXCR3 function to an individual in need thereof.

The invention also relates to a method of treating (including prophylaxis) an individual having a disease associated with pathogenic leukocyte recruitment and/or activation, such as the inflammatory and autoimmune diseases discussed herein. The method comprises administering to the individual a therapeutically effective amount of a compound or small organic molecule which is an antagonist of chemokine receptor function. Compounds or small organic molecules which have been identified as antagonists of chemokine receptor function are discussed in detail herein, and can be used for the manufacture of a medicament for treating or for preventing a disease associated with pathogenic leukocyte recruitment and/or activation.

The invention also relates to the compounds and small organic molecules described herein for use in therapy (including prophylaxis) or diagnosis, and to the use of such a compound or small organic molecule for the manufacture of a medicament for the treatment of a particular disease or condition as described herein (e.g., inflammatory disease, autoimmune disease, allergic disease, graft rejection, cancer).

The invention also includes pharmaceutical compositions comprising one or more of the compounds or small organic molecules which have been identified herein as antagonists of chemokine function and a suitable pharmaceutical carrier. The invention further relates to novel compounds which can be used to treat an individual with a disease associated with inflammation and/or pathogenic leukocyte recruitment and/or activation.

### BRIEF DESCRIPTION OF THE DRAWINGS

FIG. 1 is a schematic showing the preparation of compounds represented by Structural Formula (VI).

FIG. 2 is a schematic showing the preparation of compounds represented by Structural Formula (X).

FIG. 3 is a schematic showing the preparation of compounds represented by Structural Formula (XIV).

FIG. 4 is a schematic showing the preparation of compounds represented by Structural Formula (I).

FIG. 5 is a schematic showing the preparation of compounds represented by Structural Formula (XV).

FIG. 6 is a schematic showing the preparation of compounds represented by Structural Formula (I).

FIG. 7 is a schematic showing the preparation of compounds represented by Structural Formula (XVI).

FIG. 8 is a schematic showing the preparation of compounds represented by Structural Formula (I).

### DETAILED DESCRIPTION OF THE INVENTION

The present invention relates to small organic compounds which modulate chemokine receptor activity and are useful in the prevention or treatment of certain autoimmune or inflammatory diseases or conditions, including, for example, rheumatoid arthritis, psoriasis, and multiple sclerosis.

Specifically, the present invention relates to imidazolidine derivatives represented by Structural Formula (I):

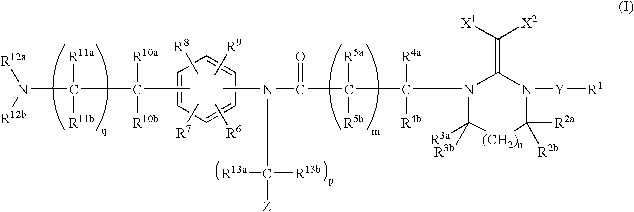

and physiologically or pharmaceutically acceptable salts thereof, wherein:

Z is

hydrogen,

halogen,

hydroxy,

—COOH,

—CONH₂,

substituted or unsubstituted lower alkyl,

substituted or unsubstituted haloalkyl,

substituted or unsubstituted heteroalkyl,

substituted or unsubstituted cycloalkyl,

substituted or unsubstituted polycycloalkyl,

substituted or unsubstituted lower alkenyl,

substituted or unsubstituted cycloalkenyl,

substituted or unsubstituted polycycloalkenyl,

substituted or unsubstituted lower alkoxy,

substituted or unsubstituted lower alkanoyloxy,

substituted or unsubstituted lower alkanoyl,

substituted or unsubstituted lower alkoxycarbonyl,

substituted or unsubstituted aralkyl,

substituted or unsubstituted heteroaralkyl,

substituted or unsubstituted aryl,

substituted or unsubstituted heteroaryl, or

a substituted or unsubstituted non-aromatic heterocyclic group, or

Z and R⁶ taken together form a bond, or

Z and R^{13a} taken together form a bond;

X¹ and X² are each, independently,

hydrogen,

—CN,

—NO₂,

—SO₂R^{15a},

—SO₂NR^{15a}R^{15b},

—C(═O)—R^{15a},

—C(═O)—OR^{15a}, or

—C(═O)—NR^{15a}R^{15b}, wherein

R^{15a} and R^{15b} are each, independently,

hydrogen,

substituted or unsubstituted lower alkyl,

substituted or unsubstituted cycloalkyl,

substituted or unsubstituted aryl, or

substituted or unsubstituted aralkyl;

Y is

a bond,

—(C═O)—, or

—(CR^{16a}R^{16b})—, wherein

R^{16a} and R^{16b} are each, independently,

hydrogen,

substituted or unsubstituted lower alkyl,

substituted or unsubstituted cycloalkyl,

substituted or unsubstituted aryl, or

substituted or unsubstituted aralkyl;

R¹ is

substituted or unsubstituted lower alkyl,

substituted or unsubstituted cycloalkyl,

substituted or unsubstituted polycycloalkyl,

substituted or unsubstituted lower alkenyl,

substituted or unsubstituted cycloalkenyl,

substituted or unsubstituted lower alkoxy,

substituted or unsubstituted lower alkanoyloxy,

substituted or unsubstituted aralkyl,

substituted or unsubstituted heteroaralkyl,

substituted or unsubstituted aryl,

substituted or unsubstituted heteroaryl, or

a substituted or unsubstituted non-aromatic heterocyclic group;

R^{2a}, R^{2b}, R^{3a}, R^{3b}, R^{4a}, R^{4b}, R^{5a}, and R^{5b} are each, independently,

hydrogen,

substituted or unsubstituted lower alkyl,

substituted or unsubstituted cycloalkyl,

substituted or unsubstituted aryl,

substituted or unsubstituted aralkyl, or

substituted or unsubstituted heteroaralkyl;

R⁶, R⁷, R⁸, and R⁹ are each, independently,

hydrogen,

hydroxy,

substituted or unsubstituted lower alkyl,

substituted or unsubstituted lower alkoxy,

substituted or unsubstituted lower alkanoyl,

substituted or unsubstituted lower alkanoyloxy,

substituted or unsubstituted lower alkoxycarbonyl,

substituted or unsubstituted aryl,

substituted or unsubstituted heteroaryl,

halogen,

—CN,

—NO₂,

—C(═O)—OR^{17a},

—NR^{17a}R^{17b}, or

—C(═O)—NR^{17a}R^{17b}, wherein

R^{17a} and R^{17b} are each, independently,

hydrogen,

substituted or unsubstituted lower alkyl,

substituted or unsubstituted cycloalkyl,

substituted or unsubstituted aryl, or

substituted or unsubstituted aralkyl, or

R^{17a} and R^{17b} taken together with the nitrogen atom to which they are bonded form a substituted or unsubstituted heterocyclic group containing at least one nitrogen atom;

R^{10a}, R^{10b}, R^{11a}, and R^{11b} are each, independently,

hydrogen,

substituted or unsubstituted lower alkyl,

substituted or unsubstituted cycloalkyl,

substituted or unsubstituted aryl,

substituted or unsubstituted aralkyl,

substituted or unsubstituted heteroaralkyl, or

substituted or unsubstituted lower alkoxyalkyl;

R^{12a} and R^{12b} are each, independently,

hydrogen,

substituted or unsubstituted lower alkyl,

substituted or unsubstituted cycloalkyl,

substituted or unsubstituted aryl,

substituted or unsubstituted aralkyl, or

substituted or unsubstituted heteroaralkyl, or

R^{12a} and R^{12b} taken together with the nitrogen atom to which they are bonded form a substituted or unsubstituted heterocyclic group containing at least one nitrogen atom;

R^{13a} and R^{13b} are each, independently,

hydrogen,

substituted or unsubstituted lower alkyl,

substituted or unsubstituted cycloalkyl,

substituted or unsubstituted aryl,

substituted or unsubstituted aralkyl, or

substituted or unsubstituted heteroaralkyl,

wherein when p is 2 or more, multiple R^{13a}'s are independently the same or different and multiple R^{13b}'s are independently the same or different;

m is an integer from 0 to 4;

n is an integer from 0 to 6;

p is an integer from 0 to 9; and

q is an integer from 0 to 5

Hereinafter, the compound(s) represented by Formula (I) are referred to as Compound(s) (I). The same applies to the compounds of other formula numbers.

As used herein, the term “alkoxy” refers to —O-alkyl; “alkanoyloxy” refers to —O—C(O)-alkyl; “alkanoyl” refers to —C(O)-alkyl; “alkoxycarbonyl” refers to —C(O)—O-alkyl.

As used herein, the term “lower alkyl” refers to straight-chain or branched alkyl groups having from 1 to about 8 carbon atoms. Lower alkyl groups, and the lower alkyl moiety of the lower alkoxy, the lower alkanoyloxy, the lower alkanoyl, the lower alkoxycarbonyl, and the lower alkoxyalkyl include, for example, methyl, ethyl, propyl, isopropyl, butyl, isobutyl, sec-butyl, tert-butyl, pentyl, hexyl, heptyl, and octyl.

A “haloalkyl” group is an alkyl group substituted with 1 or more halogens, preferably 1 to 3 halogens.

A “heteroalkyl” group is an alkyl group containing 1 or more hetero atoms, preferably 1 hetero atom, such as nitrogen, oxygen, sulfur and the like, for example, lower alkylthio, and lower alkylamino. The “alkyl moiety” of the lower alkylthio and the lower alkylamino has the same meaning as the lower alkyl defined above.

A “cycloalkyl” group is a cyclic alkyl group having from 3 to about 10 carbon atoms, for example, cyclopropyl, cyclobutyl, cyclopentyl, cyclohexyl, cycloheptyl, cyclooctyl, cyclononyl, and cyclodecyl.

A “polycycloalkyl” group is a polycyclic alkyl group having from 4 to about 12 carbon atoms, for example, bicyclo[3.2.1]octyl, bicyclo[4.3.2]undecyl, adamantyl, and noradamantyl.

A “lower alkenyl” group is a straight-chain or branched C₂ to C₈ alkyl group having one or more carbon-carbon double bonds, for example, vinyl, 1-propenyl, allyl, methacryl, 1-butenyl, crotyl, pentenyl, isoprenyl, hexenyl, heptenyl, and octenyl.

A “cycloalkenyl” group is a cyclic alkenyl group having from 4 to about 10 carbon atoms, for example, cyclobutenyl, cyclopentenyl, cyclohexenyl, cycloheptenyl, cyclooctenyl, cyclononenyl, and cyclodecenyl.

A “polycycloalkenyl” group is a polycyclic alkenyl group having from 4 to about 12 carbon atoms, for example, 6,6-dimethylbicyclo[3.1.1]hept-2-enyl, and bicyclo[3.2.1 ]oct-2-enyl.

The term “aryl” refers to carbocyclic aromatic groups, including fused polycyclic aromatic ring systems in which a carbocyclic aromatic ring is fused to one or more other carbocyclic aromatic rings. Aryl groups include, for example, phenyl, and naphthyl.

“Aralkyl” refers to an aryl-alkyl group having from 7 to about 15 carbon atoms, for example, benzyl, phenethyl, benzhydryl, naphthylmethyl, and acenaphthenyl.

The “alkyl moiety” of the haloalkyl, the aralkyl and the heteroaralkyl has the same meaning as the lower alkyl defined above.

The “alkyl moiety” of the alkyl sulfonyl, or the hydroxyalkyl has the same meaning as the lower alkyl defined above.

The term “heteroaryl” or a “heteroaryl moiety” of the heteroaralkyl refers to aromatic heterocyclic groups, including fused polycyclic aromatic ring systems in which an aromatic heterocyclic ring is fused to one or more other aromatic rings (e.g., carbocyclic aromatic or heteroaromatic), for example, pyridyl, pyrazinyl, pyrimidinyl, pyridazinyl, quinolinyl, isoquinolinyl, phthalazinyl, quinazolinyl, quinoxalinyl, naphthyridinyl, pyrrolyl, pyrazolyl, imidazolyl, triazolyl, tetrazolyl, thienyl, furyl, thiazolyl, oxazolyl, indolyl, indazolyl, benzimidazolyl, benzotriazolyl, purinyl, phenothiazinyl, and phenoxazinyl.

A “non-aromatic heterocyclic” group or a “non-aromatic heterocyclo moiety” of the non-aromatic heteroalkyl is a cycloaliphatic group that contains one or more hetero atoms, such as nitrogen, oxygen and sulfur. A non-aromatic heterocyclic group can be unsubstituted or can be substituted with a suitable substituent. Suitable substituents for a non-aromatic heterocyclic group include those substituents described herein, including fused aromatic or non-aromatic rings. Non-aromatic heterocyclic groups suitable for use in the invention include, for example, pyrrolidinyl, piperidino, piperazinyl, morpholino, thiomorpholino, homopiperidino, homopiperazinyl, tetrahydropyridinyl, tetrahydroquinolinyl, tetrahydroisoquinolinyl, pyrrolinyl, indolinyl, benzimidazolin-2-on-1-yl, imidazolin-2-on-1-yl, piperazin-2-on-4-yl, piperazine-2,3-dion-1-yl, piperazine-2,5-dion-1-yl, 1-methylpiperazin-4-yl, 1-(2-hydroxyethyl)piperazin-4-yl, 1-(3-hydroxypropyl)piperazin-4-yl, 1-benzylpiperazin-4-yl, dioxanyl, tetrahydropyranyl, and phthalimido.

A “heterocyclic group containing at least one nitrogen atom” can be an aromatic group or a cycloaliphatic group, and includes fused polycyclic ring system in which a ring containing at least one nitrogen atom is fused to one or more other rings. Examples of heterocyclic groups which contain at least one nitrogen atom include pyrrolidinyl, piperidino, piperazinyl, morpholino, thiomorpholino, homopiperidino, homopiperazinyl, tetrahydropyridinyl, tetrahydroquinolinyl, tetrahydroisoquinolinyl, pyrrolinyl, indolinyl, benzimidazolin-2-on-1-yl, imidazolin-2-on-1-yl, piperazin-2-on-4-yl, piperazine-2,3-dion-1-yl, piperazine-2,5-dion-1-yl, 1-methylpiperazin-4-yl, 1-(2-hydroxyethyl)piperazin-4-yl, 1-(3-hydroxypropyl)piperazin-4-yl, 1-benzylpiperazin4-yl, imidazolidyl, imidazolyl, benzimidazolyl, azabenzimidazolyl, phthalimido and the like.

Halogens include fluorine, chlorine, bromine, and iodine atoms.

Suitable substituents on lower alkyl, haloalkyl, heteroalkyl, cycloalkyl, polycycloalkyl, lower alkenyl, cycloalkenyl, polycycloalkenyl, lower alkoxy, lower alkanoyloxy, lower alkanoyl, lower alkoxycarbonyl, lower alkoxyalkyl, aralkyl, heteroaralkyl, aryl, heteroaryl, a non-aromatic heterocyclic group, or a heterocyclic group containing at least one nitrogen atom include, for example, halogen, —CN, —NO₂, —CF₃, hydroxy, oxo, lower alkyl, cycloalkyl, lower alkoxy, lower alkanoyl, lower alkoxycarbonyl, substituted or unsubstituted aryl (said substituent includes halogen), aralkyl, heteroaryl, heteroaralkyl, a non-aromatic heterocyclic group, hydroxyalkyl, —COOR^{18a}, —NR^{18a}R^{18b}, and —CONR^{18a}R^{18b}.

R^{18a} and R^{18b} are each, independently, hydrogen, lower alkyl, alkyl sulfonyl, cycloalkyl, aryl, or aralkyl; or R^{18a} and R^{18b} taken together with the nitrogen atom to which they are bonded form a heterocyclic group containing at least one nitrogen atom.

When a ring (e.g., cycloalkyl, polycycloalkyl, cycloalkenyl, polycycloalkenyl, aryl, heteroaryl, aralkyl, heteroaralkyl, a non-aromatic heterocyclic group, or a heterocyclic group containing at least one nitrogen atom) is substituted with one or more other rings, the rings can be fused. For example, when a phenyl ring is substituted with dioxolane the rings can be fused to create a benzodioxolanyl group. The substituted groups described herein can have one or more substituents.

Two substituents taken together can form —OCH₂O—.

In a preferred embodiment, the compound is represented by Structural Formula (I) wherein: Z is hydrogen, halogen, hydroxy, —COOH, —CONH₂, substituted or unsubstituted lower alkyl, substituted or unsubstituted haloalkyl, substituted or unsubstituted heteroalkyl, substituted or unsubstituted cycloalkyl, substituted or unsubstituted lower alkenyl, substituted or unsubstituted alkoxy, substituted or unsubstituted alkanoyloxy, substituted or unsubstituted alkanoyl, substituted or unsubstituted alkoxycarbonyl, substituted or unsubstituted aralkyl, substituted or unsubstituted heteroaralkyl, substituted or unsubstituted aryl, substituted or unsubstituted heteroaryl, or a substituted or unsubstituted non-aromatic heterocyclic group, or Z and R⁶ taken together form a bond, or Z and R^{13a} taken together form a bond; X¹ and X² are each, independently, hydrogen, —CN, —NO₂, —C(═O)—R^{15a}, —C(═O)—OR^{15a}, or —C(═O)—NR^{15a}R^{15b}, wherein R^{15a} and R^{15b} are each, independently, hydrogen, substituted or unsubstituted lower alkyl, substituted or unsubstituted cycloalkyl, substituted or unsubstituted aryl, or substituted or unsubstituted aralkyl; R⁶, R⁷, R⁸, and R⁹ are each, independently, hydrogen, substituted or unsubstituted lower alkyl, substituted or unsubstituted lower alkoxy, substituted or unsubstituted lower alkanoyl, substituted or unsubstituted lower alkoxycarbonyl, substituted or unsubstituted aryl, substituted or unsubstituted heteroaryl, halogen, —CN, —NO₂, —C(═O)—OR^{17a}, —NR^{17a}R^{17b}, or —C(═O)—NR^{17a}R^{17b}, wherein R^{17a} and R^{17b} are each, independently, hydrogen, substituted or unsubstituted lower alkyl, substituted or unsubstituted cycloalkyl, substituted or unsubstituted aryl, or substituted or unsubstituted aralkyl, or R^{17a} and R^{17b} taken together with the nitrogen atom to which they are bonded form a substituted or unsubstituted heterocyclic group containing at least one nitrogen atom; m is an integer from 0 to about 3; n is an integer from 0 to about 3; p is an integer from 0 to about 8; and q is an integer from 0 to about 3.

In a particularly preferred embodiment, X¹ and X² are each, independently hydrogen, —CN, or —NO₂; R¹ is substituted or unsubstituted lower alkyl, substituted or unsubstituted cycloalkyl, substituted or unsubstituted lower alkenyl, substituted or unsubstituted alkoxy, substituted or unsubstituted alkanoyloxy, substituted or unsubstituted aralkyl, substituted or unsubstituted heteroaralkyl, substituted or unsubstituted aryl, substituted or unsubstituted heteroaryl, or a substituted or unsubstituted non-aromatic heterocyclic group; R^{2a}, R^{2b}, R^{3a}, R^{3b}, R^{4a}, R^{4b}, R^{5a}, and R^{5b} are each, independently, hydrogen, substituted or unsubstituted lower alkyl, substituted or unsubstituted cycloalkyl, substituted or unsubstituted aryl, or substituted or unsubstituted aralkyl; R⁶, R⁷, R⁸, and R⁹ are each, independently, hydrogen, substituted or unsubstituted lower alkyl, substituted or unsubstituted lower alkoxy, substituted or unsubstituted heteroaryl, halogen, —CN, or —NO₂; and R^{10a}, R^{10b}, R^{11a}, and R^{11b} are each, independently, hydrogen, substituted or unsubstituted lower alkyl, substituted or unsubstituted cycloalkyl, or substituted or unsubstituted aryl.

Physiologically or pharmaceutically acceptable salts of Compounds (I) include acceptable acid addition salts, metal salts, ammonium salts, and organic amine addition salts. Pharmaceutically or physiologically acceptable acid addition salts of Compounds (I) include inorganic acid addition salts such as hydrochloride, sulfate, nitrate, phosphate and the like, and organic acid addition salts such as acetate, maleate, fumarate, citrate and the like. Pharmaceutically acceptable metal salts include alkali metal salts such as sodium salts and potassium salts, alkaline earth metal salts such as magnesium salts calcium salts, aluminum salts, zinc salts and the like. Pharmaceutically acceptable ammonium salts include ammonium and tetramethylammonium; and pharmaceutically acceptable organic amine addition salts include addition salts with morpholine piperidine and the like.

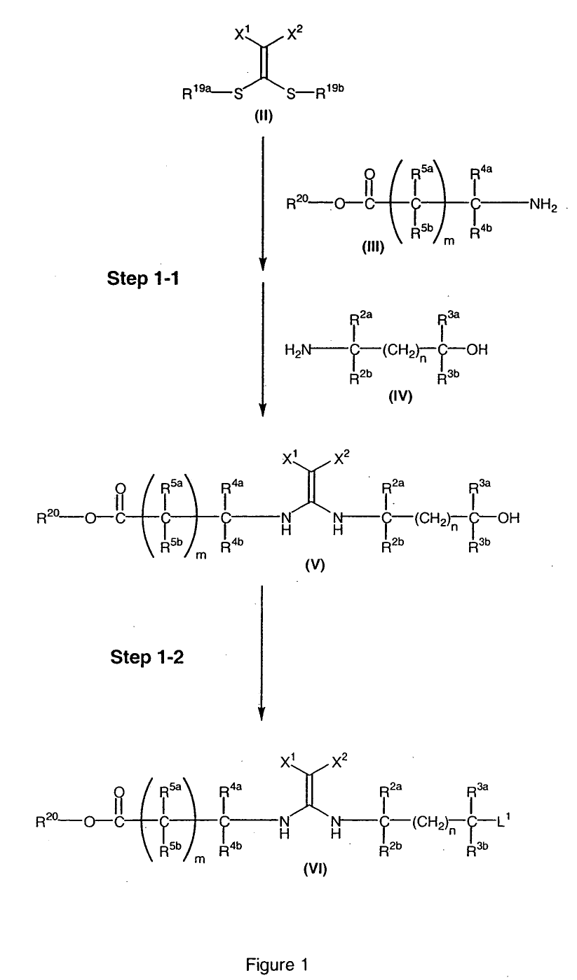

The compounds described herein can be prepared by the synthetic processes shown in FIGS. 1 to 8 described below, or by other suitable methods.

FIG. 1 is a schematic showing the preparation of compounds represented by Structural Formula (VI) by Process 1. In FIG. 1, step 1-1, R^{19a}, R^{19b} and R²⁰ are each an alkyl group. The other symbols are as defined above.

Step 1-1:

Compound (V) can be prepared by reacting Compound (II) with Compound (III) in the presence or absence of a suitable polar solvent, such as tetrahydrofuran, N,N-dimethylformamide or ethanol, at a temperature between about room temperature and about the boiling point of the solvent, evaporating the solvent, followed by adding Compound (IV) to the residue, and allowing the resulting mixture to react in the presence or absence of a suitable polar solvent, such as tetrahydrofuran, N,N-dimethylformamide or ethanol, at a temperature between about room temperature and about the boiling point of the solvent. Compound (II) can be prepared in a conventional manner using any of a variety of suitable methods known in the art. One suitable method is disclosed in Chem. Ber., vol. 95, p. 2861 (1962). The entire teachings of Chem. Ber., vol. 95, p. 2861 (1962) are incorporated herein by reference.

Step 1-2:

In FIG. 1, step 1-2, L¹ is a suitable leaving group, such as a sulfonate group (e.g., tosylate or mesylate) or a halogen atom (e.g., chlorine, bromine or iodine). The other symbols are as defined above.

Conversion of Compound (V) into Compound (VI) can be carried out using suitable methods. For example, Compound (VI) wherein L¹ is a sulfonate group can be prepared by reacting Compound (V) with a sulfonyl halide in a suitable basic solvent, e.g., pyridine, at a temperature between about 0° C. and about room temperature for about 5 minutes to about 12 hours. Compound (VI) wherein L¹ is a halogen atom can be prepared by treating Compound (V) with a halogenating agent, such as thionyl chloride, phosphorous pentachloride or phosphorous tribromide, or by allowing the above-prepared sulfonate compound to react with lithium chloride, lithium bromide, lithium iodide, or the like.

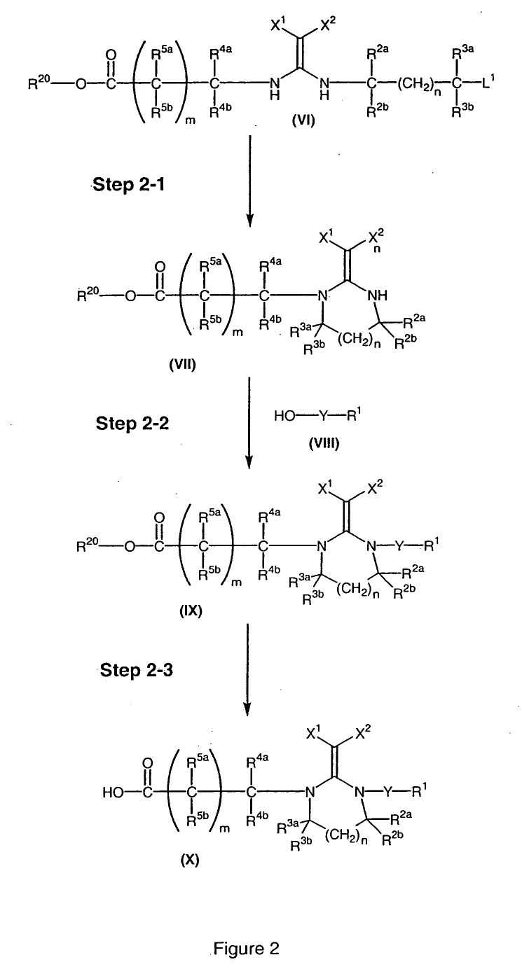

FIG. 2 is a schematic showing the preparation of compounds represented by Structural Formula (X) by Process 2. In FIG. 2, step 2-1, the symbols are as defined above.

Step 2-1:

Compound (VII) can be obtained by treating Compound (VI) with a suitable base, such as potassium tert-butoxide, sodium hydride, or 1,8-diazabicyclo[5.4.0]undec-7-ene (DBU), in a suitable solvent, such as tetrahydrofuran or N,N-dimethylformamide, at a temperature of between about 0° C. and about room temperature for about 0.5 to about 12 hours.

Step 2-2:

In FIG. 2, step 2-2, the symbols are as defined above.

Compound (IX) can be prepared by reacting Compound (VII) with Compound (VIII) using conditions described for the Mitsunobu reaction (see Carey, F. A., Sundberg, R. J. (Eds.), Advanced Organic Chemistry, 3rd ed., Plenum, New York (1990)). For example, Compound (VII) and Compound (VIII) can be treated with triphenylphosphine and diethyl azodicarboxylate in a suitable inert solvent under an inert gas atmosphere, at a temperature of between about −50° C. and about room temperature for about 5 minutes to about 48 hours to give Compound (IX).

Inert solvents suitable for use in the Mitsunobu reaction include, for example, tetrahydrofuran, dioxane, dichloromethane, toluene and benzene.

Inert gases suitable for use in the Mitsunobu reaction include, for example, argon, helium and nitrogen.

Step 2-3:

In FIG. 2, step 2-3, the symbols are as defined above.

Compound (X) can be prepared by hydrolyzing Compound (IX) in the presence of a suitable base. For example, Compound (IX) can be treated with water and a suitable base in a suitable organic solvent at a temperature between about 0° C. to about 50° C. for about 0.5 hours to about 48 hours to produce Compound (X).

Bases suitable for use in the hydrolysis include, for example, lithium hydroxide, sodium hydroxide, potassium hydroxide, barium hydroxide, sodium carbonate, potassium carbonate, and cesium carbonate. Organic solvents suitable for use in the hydrolysis include, for example, tetrahydrofuran, dioxane, methanol, ethanol, butanol, and isopropyl alcohol.

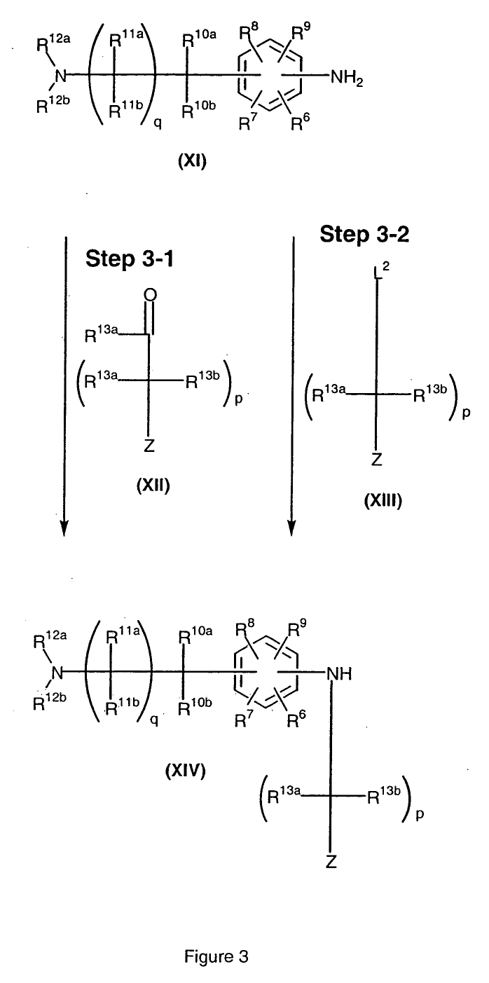

FIG. 3 is a schematic showing the preparation of compounds represented by Structural Formula (XIV) by Process 3. In FIG. 3, step 3-1, p′ is an integer from 0 to about 8, and the other symbols are as defined above.

Step 3-1:

Compound (XIV) can be obtained by reacting Compound (XI) with Compound (XII) using suitable methods in a conventional manner (see, for example, Jikken Kagaku Koza, 4th ed., vol. 20, p. 300, Maruzen (1990)). For example, Compound (XI) can be reacted with Compound (XII) in a suitable inert solvent, and the product can then be treated with a suitable reducing agent at a temperature of between about −78° C. and about the boiling point of the solvent for about 5 minutes to about 48 hours.

Solvents suitable for use in the reaction include, for example, tetrahydrofuran, dioxane, diethyl ether, ethylene glycol, dichloromethane, chloroform, methanol, ethanol, butanol, isopropyl alcohol, benzene, toluene, and water.

Reducing agents suitable for use in the reaction include, for example, lithium aluminum hydride, sodium bis(2-methoxyethoxy)aluminum hydride, potassium borohydride, sodium borohydride, sodium cyanoborohydride, sodium triacetoxyborohydride, a borane-dimethyl sulfoxide complex, a borane-dimethylamine complex, and diisobutylaluminum hydride. Compound (XI) can be prepared using suitable methods, for example, using the methods disclosed in WO99/32468.

Step 3-2:

In FIG. 3, step 3-2, L² is a suitable leaving group. The other symbols are as defined above.

Suitable leaving groups represented by L² include those defined above for the leaving groups represented by L¹.

Compound (XIV) can be prepared by reacting Compound (XI) with Compound (XIII) in a suitable inert solvent in the presence of a suitable base at a temperature of between about −50° C. and about the boiling point of the solvent for about 5 minutes to about 48 hours using suitable methods, for example, by the methods disclosed in J. Chem. Soc., 2813 (1964). If desired, Compound (XIV) can also be prepared by protecting Compound (XI) with a suitable protective group in a conventional manner (see for example Greene, T. W. and Wuts, P. G. M., Protective Groups in Organic Synthesis, 2nd ed., John Wiley & Sons, Inc., New York (1991)) and reacting the protected Compound (XI) with Compound (XIII). The protecting group can be removed from the product following the reaction in a conventional manner (see for example Greene, T. W. and Wuts, P. G. M., Protective Groups in Organic Synthesis, 2nd ed., John Wiley & Sons, Inc., New York (1991)).

Bases suitable for use in the reaction include, for example, sodium hydroxide, potassium hydroxide, sodium carbonate, potassium carbonate, sodium methoxide, potassium ethoxide, potassium tert-butoxide, butyl lithium, lithium diisopropylamide, lithium amide, triethylamine, tributylamine, N-methylmorpholine, sodium hydride, 1,8-diazabicicyclo[5.4.0]undec-7-ene (DBU), and 1,5-diazabicyclo[4.3.0]non-5-ene (DBN).

Inert solvents suitable for use in the reaction include, for example, toluene, tetrahydrofuran, dioxane, methanol, ethanol, 2-propanol, 1-butanol, dichloromethane, toluene, benzene, hexane, dimethyl sulfoxide, and N,N-dimethylformamide. Protective groups suitable for use in the reaction include, for example, a tert-butyloxycarbonyl group, a tosyl group, a 2,4-dinitrobenzenesulfonyl group, and an acetyl group.

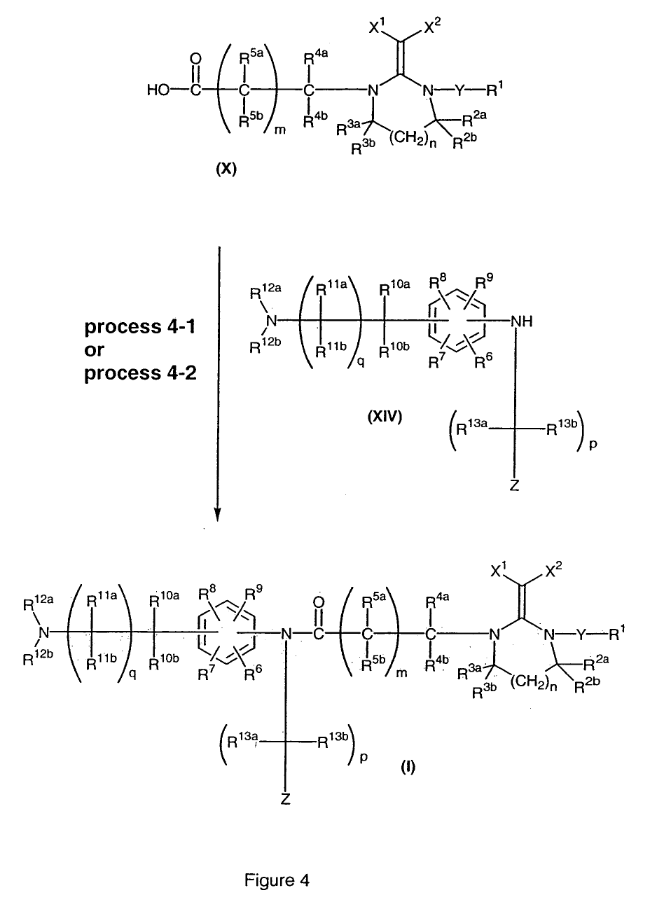

FIG. 4 is a schematic showing the preparation of compounds represented by Structural Formula (I) by Process 4. In FIG. 4, step 4-1, the symbols are as defined above.

Step 4-1:

Compound (I) can be prepared by reacting Compound (X) with Compound (XIV) in a suitable-organic solvent in the presence of a suitable condensing reagent and a suitable base at a temperature between about 0° C. and about 50° C. for between about 5 minutes and about 48 hours.

Organic solvents suitable for use in the reaction include, for example, tetrahydrofuran, dioxane, dichloromethane, N,N-dimethylformamide, and dimethyl sulfoxide. Condensing reagents suitable for use in the reaction include, for example, dicyclohexylcarbodiimide, 1-(3-dimethylaminopropyl)-3-ethylcarbodiimide, diethylphosphoric cyanide, and benzotriazol-1-yloxy-tris(dimethylamino)phosphonium hexafluorophosphate.

Bases suitable for use in the reaction include, for example, triethylamine, diisopropylethylamine, N-methylmorpholine, 1-hydroxy-7-azabenzotriazole, and 1-hydroxybenzotriazole.

Step 4-2:

In FIG. 4, step 4-2, the other symbols are as defined above.

Compound (I) can also prepared by reacting Compound (X) with a suitable halogenating agent, such as thionyl chloride, phosphorus pentachloride or phosphorus tribromide, and allowing the product to react with Compound (XIV) in a suitable polar solvent in the presence of a base at a temperature between about 0° C. and about 50° C. for about 5 minutes to about 48 hours.

Polar solvents suitable for use in the reaction include, for example, tetrahydrofuran, dioxane, N,N-dimethylformamide, and dimethyl sulfoxide.

Bases suitable for the reaction include, for example, sodium hydroxide, potassium hydroxide, sodium carbonate, potassium carbonate, sodium methoxide, potassium ethoxide, potassium tert-butoxide, butyl lithium, lithium diisopropylamide, sodium hydride, 1,8-diazabicyclo[5.4.0]undec-7-ene (DBU), 1,5-diazabicyclo[4.3.0]non-5-ene (DBN), and triethylamine.

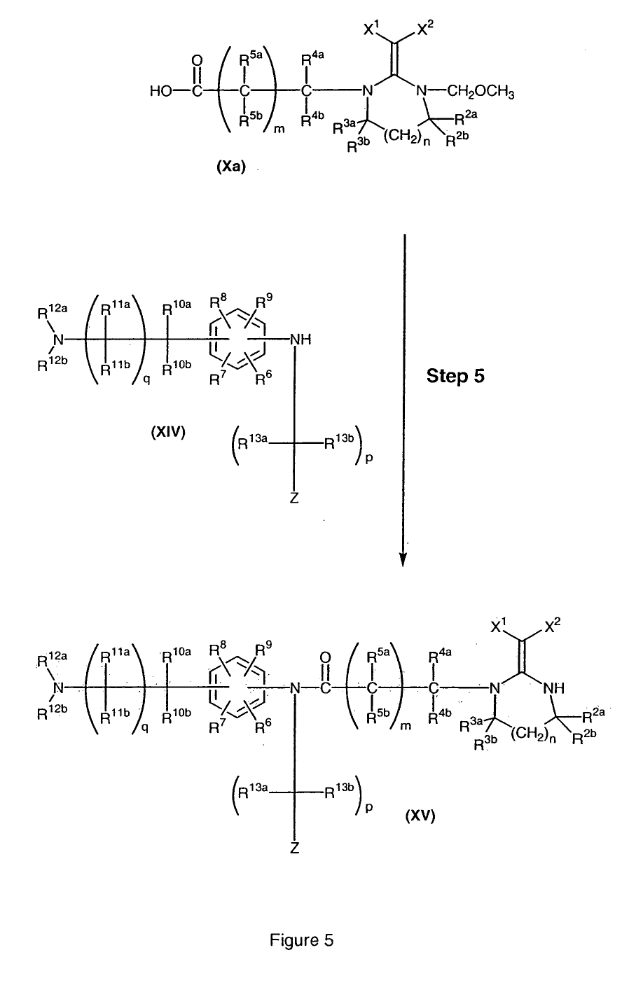

FIG. 5 is a schematic showing the preparation of compounds represented by Structural Formula (XV) by Process 5. In FIG. 5, step 5, the other symbols are as defined above.

Step 5:

Compound (XV) can be prepared from Compound (Xa), which is Compound (X) obtained in step 2-3, in which —Y—R¹ is —CH₂O—CH₃ as described in step 4-2.

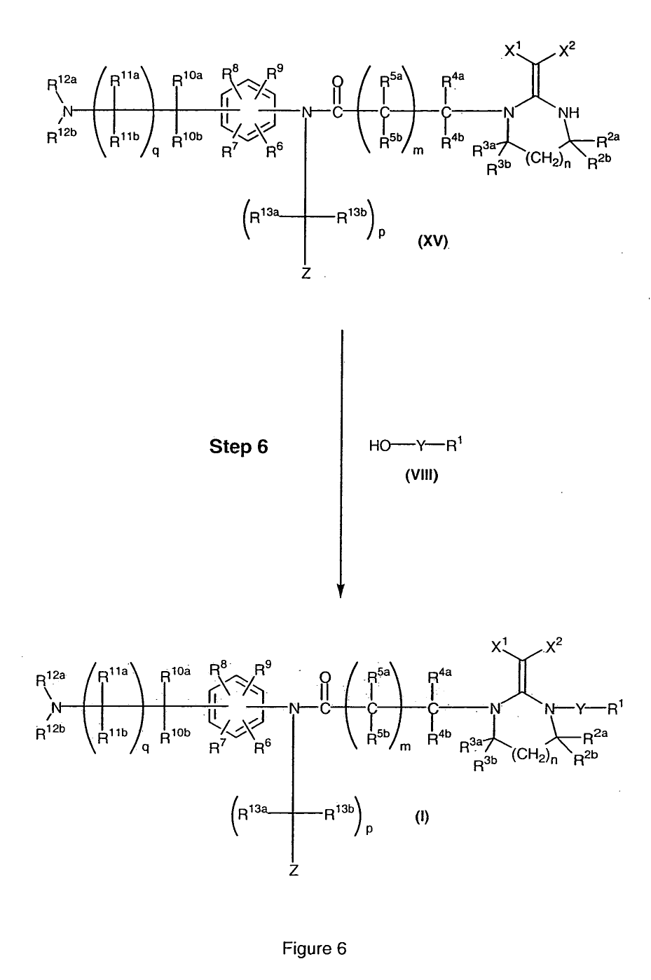

FIG. 6 is a schematic showing the preparation of compounds represented by Structural Formula (I) by Process 6. In FIG. 6, step 6, the other symbols are as defined above.

Step 6:

Compound (I) can be prepared using Compound (XV), obtained in step 5, and Compound (VIII) as described in step 2-2.

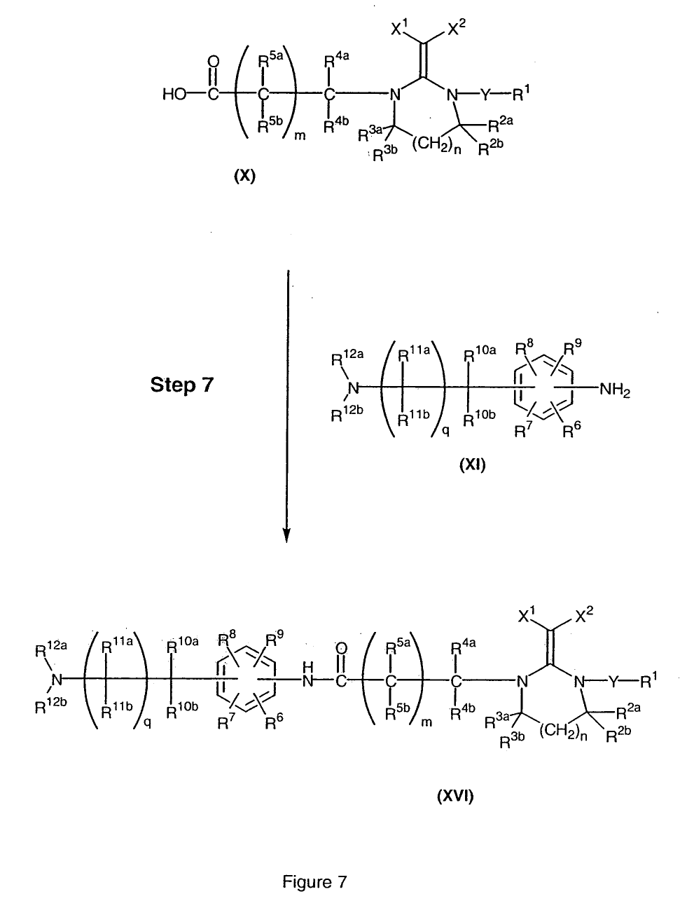

FIG. 7 is a schematic showing the preparation of compounds represented by Structural Formula (XVI) by Process 7. In FIG. 7, step 7, the other symbols are as defined above.

Step 7:

Compound (XVI) can be prepared by reacting Compound (X), obtained in step 2-3, with Compound (M) using the method described in step 4-1.

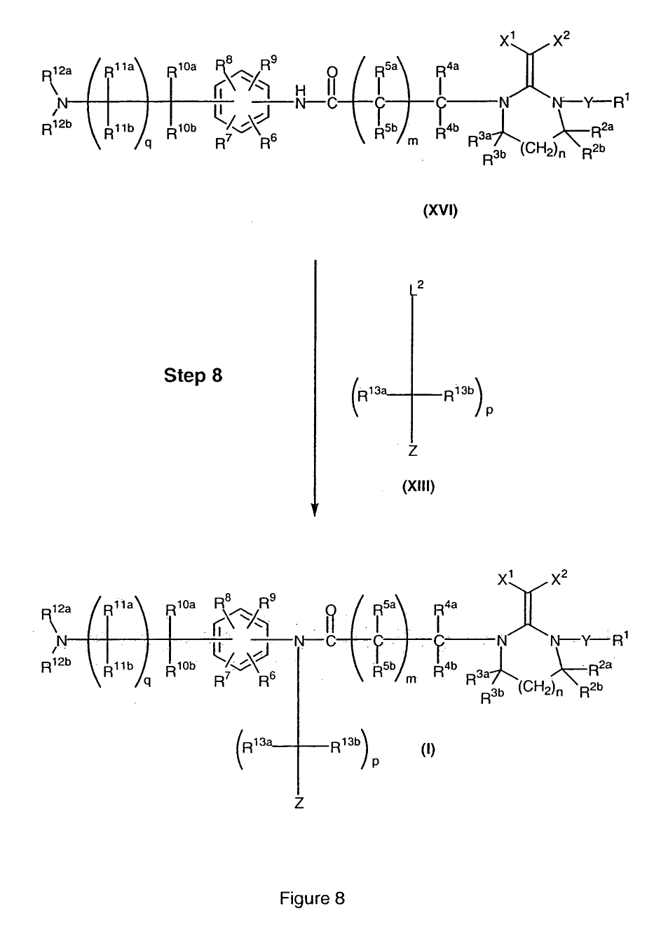

FIG. 8 is a schematic showing the preparation of compounds represented by Structural Formula (I) by Process 8. In FIG. 8, step 8, the other symbols are as defined above.

Step 8:

Compound (I) can be prepared by reacting Compound (XVI) obtained in step 7 with Compound (XIII) as described in step 3-2.

The Z group of Compound (I) can be converted to other desired groups through well-known organic chemical techniques. For example, a protective group can be removed using suitable methods, for example, using the methods disclosed in Greene, T. W., Protective Groups in Organic Synthesis, John Wiley & Sons, Inc., New York (1991). Further, COOC₂H₅ can be converted to C(CH₃)₂OH using a Grignard reagent.

The intermediates and products produced by the processes described herein can be isolated using suitable methods, for example, filtration, extraction, washing, drying, concentration, recrystallization and various kinds of chromatography. The intermediates can be subjected to subsequent reactions without isolation.

The compounds of the invention can be produced as salts or as free compounds. The desired salt of a compound of the invention can be prepared, for example, by dissolving or suspending the compound in a suitable solvent and adding a suitable acid or base to the solution, thereby forming a salt. When the compound is produced as a salt, it can be purified as such.

Compound (I) and physiologically or pharmaceutically acceptable salts thereof can be in the form of adducts with water or various solvents, which are also within the scope of the present invention.

The activity of the compounds of the present invention can be assessed using a suitable assay, such as a receptor binding assay, a chemotaxis assay, an extracellular acidification assay or a calcium flux assay (see, for example, Hesselgesser et al., J. Biol. Chem., 273(25): 15687-15692 (1998) and WO 98/02151). For example, as described herein, small organic molecule antagonists of CXCR3/IP-10 binding have been identified utilizing cells engineered to express recombinant human CXCR3 (CXCR3.L1/2) and which bind ¹²⁵I-IP-10 and chemotax in response to IP-10, Mig, or I-TAC. Specifically, a high through-put receptor binding assay, which monitors ¹²⁵I-IP-10 biding to CXCR3.L1/2 cell membranes, was used to identify small molecule antagonists. Binding assays can be performed using other ligands of CXCR3, such as, Mig, and/or I-TAC.

The activity of the compounds can also be assessed by monitoring cellular responses induced by active receptor, using suitable cells expressing receptor. For instance, exocytosis (e.g., degranulation of cells leading to release of one or more enzymes or other granule components, such as esterases (e.g., serine esterases), perforin, and/or granzymes), inflammatory mediator release (such as release of bioactive lipids such as leukotriens (e.g., leukotriene C₄)), and respiratory burst, can be monitored by methods known in the art or other suitable methods (see e.g., Taub, D. D. et al., J. Immunol., 155: 3877-3888 (1995), regarding assays for release of granule-derived serine esterases; Loetscher et al., J. Immunol., 156: 322-327 (1996), regarding assays for enzyme and granzyme release; Rot, A. et al., J. Exp. Med., 176: 1489-1495 (1992), regarding respiratory burst; Bischoff, S. C. et al., Eur. J. Immunol., 23: 761-767 (1993) and Baggliolini, M. and C. A. Dahinden, Immunology Today, 15: 127-133 (1994)).

In one embodiment, an antagonist of CXCR3 is identified by monitoring the release of an enzyme upon degranulation or exocytosis by a cell capable of this function. Cells expressing CXCR3 can be maintained in a suitable medium under suitable conditions, and degranulation can be induced. The cells are contacted with an agent to be tested, and enzyme release can be assessed. The release of an enzyme into the medium can be detected or measured using a suitable assay, such as in an immunological assay, or biochemical assay for enzyme activity.

The medium can be assayed directly, by introducing components of the assay (e.g., substrate, co-factors, antibody) into the medium (e.g., before, simultaneous with or after the cells and agent are combined). The assay can also be performed on medium which has been separated from the cells or further processed (e.g., fractionated) prior to assay. For example, convenient assays are available for enzymes, such as serine esterases (see e.g., Taub, D. D. et al., J. Immunol., 155: 3877-3888 (1995) regarding release of granule-derived serine esterases).

In another embodiment, cells expressing CXCR3 are combined with a ligand of CXCR3 (e.g., IP-10, Mig, I-TAC) or promotor of CXCR3 function, a compound to be tested is added before, after or simultaneous therewith, and degranulation is assessed. Inhibition of ligand- or promoter-induced degranulation is indicative that the compound is an inhibitor of mammalian CXCR3 function (a CXCR3 antagonist).

Therapeutic Applications:

The compounds of the present invention are useful in the treatment of certain diseases or conditions (e.g., autoimmune, inflammatory, infectious, cancer). Modulation of mammalian CXCR function according to the present invention, through the inhibition or promotion of at least one function characteristic of a mammalian CXCR protein, provides an effective and selective way of inhibiting or promoting receptor-mediated functions. As CXC-chemokine receptors selectively expressed on activated lymphocytes, responsive to chemokines such as IP-10, Mig, and I-TAC whose primary targets are lymphocytes, particularly effector cells such as activated or stimulated T lymphocytes and NK cells, mammalian CXCR3 proteins provide a target for selectively interfering with or promoting lymphocyte function in a mammal, such as a human. Once lymphocytes are recruited to a site, other leukocyte types, such as monocytes, may be recruited by secondary signals. Thus, agents which inhibit or promote CXCR3 function, including ligands, inhibitors (antagonists) and/or promoters (agonists), such as the compounds described herein, can be used to modulate leukocyte function (e.g., leukocyte infiltration including recruitment and/or accumulation), particularly of lymphocytes, for therapeutic purposes.

In one aspect, the present invention is a method of modulating (inhibiting or promoting) an inflammatory response in an individual in need of such therapy, comprising administering a compound which inhibits or promotes mammalian CXCR3 function to an individual in need of such therapy. In one embodiment, a compound which inhibits one or more functions of a mammalian CXCR3 protein (e.g., a human CXCR3) is administered to inhibit (i.e., reduce or prevent) inflammation. For example, the small organic molecules of the present invention, including compound (I), can be used in the method. As a result, one or more inflammatory processes, such as leukocyte emigration, chemotaxis, exocytosis (e.g., of enzymes) or inflammatory mediator release, can be inhibited. For example, leukocytic infiltration of inflammatory sites (e.g., in a delayed-type hypersensitivity response) can be inhibited according to the present method. The inflammation can be a consequence of an autoimmune disease, allergic reaction, infection (e.g., bacterial, viral, fungal, parasitic) or trauma (e.g., ischemia/reperfusion injury), for example.

In another embodiment, a compound (e.g., receptor agonist) which promotes one or more functions of a mammalian CXCR3 protein (e.g., a human CXCR3) is administered to induce (trigger or enhance) an inflammatory response, such as leukocyte emigration, chemotaxis, exocytosis (e.g., of enzymes) or inflammatory mediator release, resulting in the beneficial stimulation of inflammatory processes. For example, natural killer cells can be recruited to combat viral infections or neoplastic disease.

In another embodiment, the present invention is a method of treating (e.g., palliative therapy, curative therapy, maintenance therapy, prophylactic therapy) an individual having a disease associated with pathogenic leukocyte recruitment and/or activation. The method comprising administering a compound which inhibits mammalian CXCR3 function (e.g., a compound of Structural Formula (I) or physiologically or pharmaceutically acceptable salts thereof) to an individual in need of such therapy. Where the individual has a relapsing or chronic condition, an effective amount of a compound which inhibits mammalian CXCR3 function (e.g., a compound of Structural Formula I or physiologically or pharmaceutically acceptable salt thereof) can be administered to treat the condition, and therapy can be continued (maintenance therapy) with the same or different dosing as indicated, to inhibit relapse or renewed onset of symptoms.

The term “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species. Diseases and conditions associated with inflammation, infection, and cancer can be treated using the method. In a preferred embodiment, the disease or condition is one in which the actions of lymphocytes, particularly effector cells such as activated or stimulated T lymphocytes and natural killer (NK) cells, are to be inhibited or promoted for therapeutic (including prophylactic) purposes. In a particularly preferred embodiment, the inflammatory disease or condition is a T cell-mediated disease or condition.

Diseases or conditions, including acute and/or chronic diseases, of humans or other species which can be treated with inhibitors of CXC chemokine receptor 3 (CXCR3) function, include, but are not limited to:

inflammatory or allergic diseases and conditions, including systemic anaphylaxis or hypersensitivity responses, drug allergies (e.g., to penicillin, cephalosporins), insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis (e.g., necrotizing, cutaneous, and hypersensitivity vasculitis); spondyloarthropathies; scleroderma; respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, or other autoimmune conditions);

autoimmune diseases, such as arthritis (e.g., rheumatoid arthritis, psoriatic arthritis), multiple sclerosis, systemic lupus erythematosus, myasthenia gravis, diabetes, including diabetes mellitus and juvenile onset diabetes, glomerulonephritis and other nephritides, autoimmune thyroiditis, Behcet's disease;

graft rejection (e.g., in transplantation), including allograft rejection or graft-versus-host disease;

other diseases or conditions in which undesirable inflammatory responses are to be inhibited can be treated, including, but not limited to, atherosclerosis, restinosis, cytokine-induced toxicity, myositis (including polymyositis, dermatomyositis);

diseases in which angiogenesis or neovascularization plays a role, including neoplastic disease (e.g., tumor formation and growth), retinopathy (e.g., retinopathy of prematurity, diabetic retinopathy), and macular degeneration (e.g., age related macular degradation), hemangiomas, arthritis (e.g., rheumatoid arthritis) and psoriasis.

Diseases or conditions of humans or other species which can be treated with a promoter (e.g., an agonist) of CXCR3 function, include, but are not limited to:

cancers, particularly those with leukocytic infiltration of the skin or organs such as cutaneous T cell lymphoma (e.g., mycosis fungoides);

diseases in which angiogenesis or neovascularization plays a role, including neoplastic disease, retinopathy (e.g., diabetic retinopathy), and macular degeneration;

infectious diseases, such as bacterial infections and tuberculoid leprosy, and especially viral infections;

immunosuppression, such as that in individuals with immunodeficiency syndromes such as AIDS, and that in individuals undergoing radiation therapy, chemotherapy, or other therapy which causes immunosuppression; immunosuppression due to congenital deficiency in receptor function or other causes. Promoters of CXCR3 function can also have protective effects useful to combat stem cell depletion during cancer chemotherapy (Sarris, A. H. et al., J. Exp. Med., 178: 1127-1132 (1993)).

Modes of Administration:

According to the method, one or more compounds can be administered to an individual by an appropriate route, either alone or in combination with another drug. A therapeutically effective amount of an agent (e.g., a small organic molecule which inhibits ligand binding) is administered.

A “therapeutically effective amount” of a compound is an amount which is sufficient to achieve a desired therapeutic and/or prophylactic effect, such an amount which results in the prevention or a decrease in the severity of symptoms associated with an inflammatory disease or condition. For example, an effective amount of an antagonist of CXCR3 function is an amount sufficient to inhibit a (i.e., one or more) function of CXCR3 (e.g., ligand (e.g., IP-10, Mig, I-TAC) binding, ligand-induced leukocyte migration, ligand-induced integrin activation, ligand-induced transient increases in the concentration of intracellular free calcium [Ca²⁺]ᵢ and ligand-induced granule release of proinflammatory mediators).

The amount of compound administered to the individual will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, a therapeutically effective amount of the compound can range from about 0.1 mg per day about 100 mg per day for an adult. Preferably, the dosage ranges from about 1 mg per day to about 100 mg per day. An antagonist of chemokine receptor function can also be administered in combination with one or more additional therapeutic agents, e.g., theophylline, b-adrenergic bronchdilators, corticosteroids, antihistamines, antiallergic agents, immunosuppressive agents and the like.

The compound of the invention can be administered by any suitable route, including, for example, orally in capsules, suspensions or tablets or by parenteral administration. Parenteral administration can include, for example, intramuscular, intravenous, subcutaneous, or intraperitoneal administration. The compound can also be administered orally (e.g., dietary), transdermally, topically, by inhalation (e.g., intrabronchial, intranasal, oral inhalation or intranasal drops) or rectally. Administration can be local or systemic as indicated. The preferred mode of administration can vary depending upon the particular disease or condition to be treated, however, oral or parenteral administration is generally preferred.

The compound can be administered to the individual in conjunction with a pharmaceutically acceptable carrier as part of a pharmaceutical composition for treatment (e.g., palliative therapy, curative therapy, maintenance therapy, prophylactic therapy) or prevention of inflammation, an inflammatory disease or other disease (e.g., an autoimmune disease), as described herein. Formulation of a compound to be administered will vary according to the route of administration selected (e.g., solution, emulsion, capsule). Suitable pharmaceutically acceptable carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutically acceptable carriers for parenteral administration include, for example, sterile water, physiological saline, bacteriostatic saline (saline containing about 0.9% mg/mL benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986).

The compounds of the present invention can also be administered to treat inflammatory and/or autoimmune diseases and/or conditions in combination with a variety of other anti-inflammatory and/or immunosuppressive drugs, such as cyclosporin A, steroids (e.g., prednisone, methylprednisolone), azothioprine, methotrexate, or FK506 (tacrolimus). Such combination therapy can result in more efficacious therapy with reduced doses of the anti-inflammatory or immunosuppressive drugs. The ability to reduce the dose of the anti-inflammatory or immunosuppressive drug can greatly benefit the patient as many of these drugs have severe and well-known side effects (Spencer, C. M. et al., Drugs, 54(6): 925-075 (1997); Physicians Desk Reference, 53^{rd} Edition, Medical Economics Co., pp. 2081-2082 (1999)).

The invention is illustrated by the following Examples, Reference Examples and Test Examples which are not intended to be limiting in any way.

Exemplification

### EXAMPLE 1

### N-Methyl-N-[3-(piperidinomethyl)phenyl]-3-[3-(3,5-dimethylbenzyl)-2-dicyanomethylidene imidazolidin-1-yl]propionamide (Compound 1)

Thionyl chloride (2.0 mL) was added to Compound B (0.20 g) obtained in Reference Example 2, followed by stirring at room temperature for 20 minutes. Thionyl chloride was evaporated under reduced pressure. Toluene was added thereto to cause azeotropy to give a crude acid chloride. Separately, Compound E (0.14 g) obtained in Reference Example 5 was dissolved in tetrahydrofuran (1.0 mL), and triethylamine (0.42 mL) was added thereto, followed by stirring at room temperature for 5 minutes. To the resulting mixture was added dropwise a solution of the above prepared acid chloride in tetrahydrofuran (2.0 mL), followed by stirring at room temperature for 12 hours. A saturated aqueous sodium bicarbonate solution was added thereto, followed by extraction with chloroform. The extract was dried over anhydrous sodium sulfate, and the solvent was evaporated under reduced pressure. The residue was purified by silica gel preparative thin layer chromatography (hexane:ethylacetate:triethylamine=5:10:1) to give Compound 1(0.12 g, 37%) as a pale yellow oily substance.

¹H NMR (270 MHz, CDCl₃) δ 7.45-7.30 (2H, m), 7.24 (1H, brs), 7.08 (1H, brd), 6.95 (1H, brs), 6.86 (2H, brs), 4.64 (2H, s), 3.90-3.74 (2H, m), 3.77-3.61 (2H, m), 3.55 (2H, s), 3.48-3.32 (2H, m), 3.27 (3H, s), 2.61-2.45 (2H, m), 2.44 (4H, m), 2.30 (6H, s), 1.61 (4H, m), 1.46 (2H, m). MASS (m/e) 511 [(M+H)⁺]

### EXAMPLE 2

### N-Propyl-N-[3-(piperidinomethyl)phenyl]-3-[3-(3,5-dimethylbenzyl)-2-dicyanomethylidene imidazolidin-1-yl]propionamide (Compound 2)

Compound 2 (0.053 g, 16%) was obtained as a pale yellow oily substance using Compound B (0.20 g) obtained in Reference Example 2, Compound F (0.16 g) obtained in Reference Example 6, thionyl chloride (2.0 mL), triethylamine (0.42 mL), and tetrahydrofuran (3.0 mL) as described in Example 1.

¹H NMR (270 MHz, CDCl₃) δ 7.45-7.32 (2H, m), 7.23 (1H, brs), 7.05 (1H, m), 6.95 (1H, brs), 6.86 (2H, brs), 4.64 (2H, s), 3.90-3.74 (2H, m), 3.78-3.59 (4H, m), 3.58 (2H, s), 3.48-3.32 (2H, m), 2.60-2.35 (6H, m), 2.30 (6H, s), 1.82-1.38 (8H, m), 0.89 (3H, t). MASS (m/e) 539 [(M+H)⁺]

### EXAMPLE 3

### N-Isopropyl-N-[3-(piperidinomethyl)phenyl]-3-[3-(3,5-dimethylbenzyl)-2-dicyanomethylidene imidazolidin-1-yl]propionamide (Compound 3)

Thionyl chloride (1.0 mL) was added to Compound B (0.17 g) obtained in Reference Example 2. After the solution was stirred at room temperature for 20 minutes, thionyl chloride was evaporated under reduced pressure. Toluene was added thereto to cause azeotropy to give a crude acid chloride. Separately, Compound G (0.15 g) obtained in Reference Example 7 was dissolved in tetrahydrofuran (1.0 mL), and a 60% dispersion (0.052 g) of sodium hydride in mineral oil was added thereto, followed by stirring at room temperature for 10 minutes. After ice-cooling, a solution of the above prepared acid chloride in tetrahydrofuran (1.0 mL) was added dropwise thereto, followed by stirring at room temperature for 20 minutes. After ice-cooling, a saturated aqueous sodium bicarbonate solution was added thereto, followed by extraction with chloroform. The extract was dried over anhydrous sodium sulfate, and the solvent was evaporated under reduced pressure. The residue was purified by silica gel preparative thin layer chromatography (hexane:ethyl acetate:triethylamine=5:10:1) to give Compound 3 (0.17 g, 61%) as a pale yellow oily substance.

¹H NMR (270 MHz, CDCl₃) δ 7.48-7.30 (2H, m), 7.15 (1H, brs), 7.00 (1H, m), 6.95 (1H, brs), 6.86 (2H, brs), 4.95 (1H, septet), 4.74-4.52 (2H, m), 3.88-3.72 (2H, m), 3.76-3.58 (2H, m), 3.53 (2H, brs), 3.48-3.30 (2H, m), 2.52-2.22 (6H, m), 2.30 (6H, s), 1.58 (4H, m), 1.45 (2H, m), 1.06 (6H, d). MASS (m/e) 539 [(M+H)⁺]

### EXAMPLE 4

### N-Cyclohexyl-N-[3-(piperidinomethyl)phenyl]-3-[3-(3,5-dimethylbenzyl)-2-dicyanomethylidene imidazolidin-1-yl]propionamide (Compound 4)

Compound 4 (0.18 g, 60%) was obtained as a pale yellow oily substance using Compound B (0.17 g) obtained in Reference Example 2, Compound H (0.17 g) obtained in Reference Example 8, thionyl chloride (1.0 mL), and a 60% dispersion (0.052 g) of sodium hydride in mineral oil as described in Example 3.

¹H NMR (270 MHz, CDCl₃) δ 7.44-7.30 (2H, m), 7.14 (1H, brs), 6.98 (1H, m), 6.94 (1H, brs), 6.86 (2H, brs), 4.74-4.52 (2H, m), 4.53 (1H, m), 3.88-3.72 (2H, m), 3.76-3.58 (2H, m), 3.64-3.42 (2H, m), 3.48-3.30 (2H, m), 2.50-2.24 (6H, m), 2.30 (6H, s), 1.92-0.78 (16H, m). MASS (m/e) 579 [(M+H)⁺]

### EXAMPLE 5

### N-Benzyl-N-[3-(piperidinomethyl)phenyl]-3-[3-(3,5-dimethylbenzyl)-2-dicyanomethylidene imidazolidin-1-yl]propionamide (Compound 5)

Compound 5 (0.014 g, 4%) was obtained as a pale yellow oily substance using Compound B (0.20 g) obtained in Reference Example 2, Compound I (0.19 g) obtained in Reference Example 9, thionyl chloride (2.0 mL), triethylamine (0.42 mL), and tetrahydrofuran (3.0 mL) as described in Example 1.

¹H NMR (270 MHz, CDCl₃) δ 7.40-7.10 (7H, m), 7.04-6.80 (5H, m), 4.87 (2H, s), 4.64 (2H, s), 3.93-3.77 (2H, m), 3.76-3.58 (2H, m), 3.47 (2H, brs), 3.44-3.28 (2H, m), 2.61-2.45 (2H, m), 2.32 (4H, m), 2.31 (6H, s), 1.56 (4H, m), 1.42 (2H, m). MASS (m/e) 587 [(M+H)⁺]

### EXAMPLE 6

### N-(2-Acetoxyethyl)-N-[3-(piperidinomethyl)phenyl]-3-[3-(3,5-dimethylbenzyl)-2-dicyanomethylidene imidazolidin-1-yl]propionamide (Compound 6)

Compound J (0.10 g) obtained in Reference Example 10 was dissolved in dimethyl sulfoxide (0.40 mL), and 2-bromoethyl acetate (0.40 mL) and potassium hydroxide (0.016 g) were added thereto, followed by stirring at room temperature for 30 minutes. To the reaction mixture were further added 2-bromoethyl acetate (0.20 mL), potassium hydroxide (0.016 g), and dimethyl sulfoxide (0.20 mL), followed by stirring at room temperature for 1.5 hours. Water was added thereto, followed by extraction with ethyl acetate. The extract was dried over anhydrous sodium sulfate, and the solvent was evaporated under reduced pressure. The residue was purified by silica gel preparative thin layer chromatography (chloroform:methanol=10:1) to give Compound 6 (0.015 g, 12%) as a pale yellow oily substance.

¹H NMR (270 MHz, CDCl₃) δ 7.48-7.30 (2H, m), 7.23 (1H, brs), 7.08 (1H, m), 6.95 (1H, brs), 6.86 (2H, brs), 4.64 (2H, s), 4.23 (2H, t), 3.95 (2H, t), 3.81 (2H, t), 3.77-3.61 (2H, m), 3.53 (2H, brs), 3.47-3.31 (2H, m), 2.51 (2H, t), 2.41 (4H, m), 2.31 (6H, s), 1.98 (3H, s), 1.60 (4H, m), 1.46 (2H, m). MASS (m/e) 583 [(M+H)⁺]

### EXAMPLE 7

### N-(2-Hydroxyethyl)-N-[3-(piperidinomethyl)phenyl]-3-[3-(3,5-dimethylbenzyl)-2-dicyanomethylidene imidazolidin-1-yl]propionamide (Compound 7)

Compound 6 (0.015 g) obtained in Example 6 was dissolved in tetrahydrofuran (0.10 mL), and a 0.5 mol/L aqueous lithium hydroxide solution (0.10 mL) and methanol (0.050 mL) were added thereto, followed by stirring at room temperature for 30 minutes. A saturated aqueous sodium bicarbonate solution was added thereto, followed by extraction with chloroform. The extract was dried over anhydrous sodium sulfate, and the solvent was evaporated under reduced pressure. The residue was purified by silica gel preparative thin layer chromatography (chloroform:methanol=10:1) to give Compound 7 (0.012 g, 90%) as a pale yellow oily substance.

¹H NMR (270 MHz, CDCl₃) δ 7.46-7.32 (2H, m), 7.28 (1H, brs), 7.12 (1H, m), 6.95 (1H, brs), 6.86 (2H, brs), 4.65 (2H, s), 3.94-3.72 (6H, m), 3.76-3.60 (2H, m), 3.53 (2H, s), 3.48-3.32 (2H, m), 2.61-2.45 (2H, m), 2.41 (4H, m), 2.31 (6H, s), 1.60 (4H, m), 1.46 (2H, m). The signal which corresponds to a hydroxyl group was not observed. MASS (m/e) 541 [(M+H)⁺]

### EXAMPLE 8

### N-Ethoxycarbonylmethyl-N-[3-(piperidinomethyl)phenyl]-3-[3-(3,5-dimethylbenzyl)-2-dicyanomethylidene imidazolidin-1-yl]propionamide (Compound 8)

Compound J (0.050 g) obtained in Reference Example 10 was dissolved in tetrahydrofuran (1.5 mL), and the solution was cooled with ice. Potassium tert-butoxide (0.017 g) was added thereto under ice-cooling, followed by stirring while ice-cooling for 30 minutes. Ethyl bromoacetate (0.017 mL) was added thereto, followed by stirring under ice-cooling for 30 minutes. A saturated aqueous sodium bicarbonate solution was added thereto, followed by extraction with chloroform. The extract was dried over anhydrous sodium sulfate, and the solvent was evaporated under reduced pressure. The residue was purified by silica gel preparative thin layer chromatography (chloroform:methanol=10:1) to give Compound 8 (0.030 g, 50%).

¹H NMR (270 MHz, CDCl₃) δ 7.46-7.34 (2H, m), 7.33 (1H, brs), 7.22 (1H, m), 6.95 (1H, brs), 6.88 (2H, brs), 4.66 (2H, s), 4.35 (2H, s), 4.18 (2H, q), 3.93-3.77 (2H, m), 3.79-3.63 (2H, m), 3.55 (2H, brs), 3.50-3.34 (2H, m), 2.68-2.52 (2H, m), 2.43 (4H, m), 2.31 (6H, s), 1.61 (4H, m), 1.46 (2H, m), 1.27 (3H, t). MASS (m/e) 583 [(M+H)⁺]

### EXAMPLE 9

### N-Carboxymethyl-N-[3-(piperidinomethyl)phenyl]-3-[3-(3,5-dimethylbenzyl)-2-dicyanomethylidene imidazolidin-1-yl]propionamide (Compound 9)

Compound 8 (0.022 g) obtained in Example 8 was dissolved in tetrahydrofuran (0.10 mL), and a 0.7 mol/L aqueous lithium hydroxide solution (0.10 mL) and methanol (0.10 mL) were added thereto, followed by stirring at room temperature for 1.5 hours. The solvent was evaporated under reduced pressure, and ethyl acetate was added thereto, followed by extraction with an aqueous potassium hydroxide solution. The pH of the aqueous layer was adjusted to about 7 by adding 1 mol/L hydrochloric acid, followed by extraction with chloroform. The extract was washed with a saturated aqueous sodium chloride solution and dried over anhydrous sodium sulfate. The solvent was evaporated under reduced pressure to give Compound 9 (0.013 g, 60%) as pale yellow crystals.

¹H NMR (270 MHz, CD₃OD) δ 7.66-7.42 (4H, m), 6.96 (1H, brs), 6.91 (2H, brs), 4.63 (2H, brs), 4.28 (2H, brs), 4.25 (2H, s), 3.88-3.72 (2H, m), 3.82-3.66 (2H, m), 3.58-3.42 (2H, m), 3.18 (4H, m), 2.68-2.52 (2H, m), 2.29 (6H, s), 1.83 (4H, m), 1.66 (2H, m). The signal which corresponds to a carboxyl group was not observed. MASS (m/e) 555 [(M+H)⁺]

### EXAMPLE 10

### N-(2-Hydroxy-2-methylpropyl)-N-[3-(piperidinomethyl)phenyl]-3-[3-(3,5-dimethylbenzyl)-2-dicyanomethylidene imidazolidiny-1-yl]propionamide (Compound 10)

Compound 8 (0.029 g) obtained in Example 8 was dissolved in tetrahydrofuran (0.50 mL), and a 0.93 mol/L solution (0.20 mL) of methylmagnesium bromide in tetrahydrofuran was added thereto, followed by stirring at room temperature for 15 minutes. A saturated aqueous sodium bicarbonate solution was added thereto, followed by extraction with chloroform. The solvent was evaporated under reduced pressure, and the residue was purified by silica gel preparative thin layer chromatography (chloroform:methanol=10:1) to give Compound 10 (0.013 g, 47%) as a pale yellow oily substance.

¹H NMR (270 MHz, CDCl₃) δ 7.42 (1H, brt), 7.33 (1H, brd), 7.28 (1H, brs), 7.11 (1H, brd), 6.95 (1H, brs), 6.86 (2H, brs), 4.65 (2H, s), 3.89-3.73 (2H, m), 3.79 (2H, s), 3.78-3.62 (2H, m), 3.53 (2H, brs), 3.47-3.31 (2H, m), 2.66-2.50 (2H, m), 2.41 (4H, m), 2.31 (6H, s), 1.59 (4H, m), 1.46 (2H, m), 1.21 (6H, s). The signal which corresponds to a hydroxyl group was not observed. MASS (m/e) 569 [(M+H)⁺]

### EXAMPLE 11

### N-(3-Fluoropropyl)-N-[3-(piperidinomethyl)phenyl]-3-[3-(3,5-dimethylbenzyl)-2-dicyanomethylidene imidazolidin-1-yl]propionamide (Compound 11)

Compound 11 (0.033 g, 54%) was obtained as a pale yellow oily substance using Compound J (0.054 g) obtained in Reference Example 10, 1-bromo-3-fluoropropane (0.22 mL), potassium hydroxide (0.014 g), and dimethyl sulfoxide (0.22 mL) as described in Example 6.

¹H NMR (270 MHz, CDCl₃) δ 7.41 (1H, brt), 7.35 (1H, brd), 7.21 (1H, brs), 7.06 (1H, brd), 6.95 (1H, brs), 6.86 (2H, brs), 4.64 (2H, s), 4.57 (1H, t), 4.40 (1H, t), 3.92-3.74 (4H, m), 3.76-3.60 (2H, m), 3.53 (2H, brs), 3.47-3.31 (2H, m), 2.57-2.41 (2H, m), 2.41 (4H, m), 2.31 (6H, s), 1.99 (1H, m), 1.90 (1H, m), 1.59 (4H, m), 1.27 (2H, m). MASS (m/e) 557 [(M+H)⁺]

### EXAMPLE 12

### N-Aminocarbonylmethyl-N-[3-(piperidinomethyl)phenyl]-3-[3-(3,5-dimethylbenzyl)-2-dicyanomethylidene imidazolidin-1-yl]propionamide (Compound 12)

Compound 12 (0.0080 g, 13%) was obtained as a pale yellow oily substance using Compound J (0.057 g) obtained in Reference Example 10, iodoacetamide (0.029 g), potassium hydroxide (0.012 g), and dimethyl sulfoxide (0.20 mL) as described in Example 6.

¹H NMR (270 MHz, CDCl₃) δ 7.74 (1H, brs), 7.54-7.24 (3H, m), 6.94 (1H, brs), 6.87 (2H, brs), 6.55 (1H, brs), 5.53 (1H, brs), 4.66 (2H, s), 4.37 (2H, brs), 3.93-3.77 (2H, m), 3.92 (2H, brs), 3.79-3.63 (2H, m), 3.51-3.35 (2H, m), 2.84 (4H, m), 2.74-2.58 (2H, m), 2.30 (6H, s), 1.87 (4H, m), 1.60 (2H, m). MASS (m/e) 557 [(M+H)⁺]

### EXAMPLE 13

### N-(1H-tetrazol-5-ylmethyl)-N-[3-(piperidinomethyl)phenyl]-3-[3-(3,5-dimethylbenzyl)-2-dicyanomethylidene imidazolidin-1-yl]propionamide (Compound 13)

Compound K (0.0082 g) obtained in Reference Example 11 was dissolved in methanol (0.070 mL), and 1 mol/L hydrochloric acid (0.020 mL) was added thereto, followed by stirring at room temperature for 1.5 hours. The solvent was evaporated under reduced pressure, and the residue was purified by silica gel preparative thin layer chromatography (chloroform:methanol=2: 1) to give Compound 13 (0.0030 g, 52%) as a pale yellow oily substance.

¹H NMR (270 MHz, CD₃OD) δ 7.40-7.26 (2H, m), 7.18-7.06 (2H, m), 6.95 (1H, brs), 6.90 (2H, brs), 5.15 (2H, s), 4.63 (2H, s), 3.91-3.75 (2H, m), 3.78-3.62 (2H, m), 3.58-3.40 (2H, m), 3.52 (2H, s), 2.63-2.47 (2H, m), 2.38 (4H, m), 2.28 (6H, s), 1.58 (4H, m), 1.44 (2H, m). The signal which corresponds to a tetrazolyl group was not observed. MASS (m/e) 579 [(M+H)⁺]

### EXAMPLE 14

### N-Isopropyl-N-[3-(piperidinomethyl)phenyl]-3-[3-(2,3-dimethylbenzyl)-2-dicyanomethylidene imidazolidin-1-yl]propionamide (Compound 14)

Compound M (0.040 g) obtained in Reference Example 13 and 2,3-dimethylbenzyl alcohol (0.11 g) were dissolved in tetrahydrofuran (0.50 mL). Triphenylphosphine (0.21 g) and diethyl azodicarboxylate (0.12 mL) were added thereto under ice-cooling, followed by stirring at room temperature for 12 hours. The solvent was evaporated under reduced pressure, and the residue was purified by silica gel preparative thin layer chromatography (hexane:ethyl acetate:triethylamine=5:10:1) to give Compound 14 (0.016 g, 32%) as a pale yellow oily substance.

¹H NMR (270 MHz, CDCl₃) δ 7.50-7.38 (2H, m), 7.30-6.94 (5H, m), 4.96 (1H, septet), 4.86-4.66 (2H, m), 3.88-3.50 (6H, m), 3.38-3.22 (2H, m), 2.52 (4H, m), 2.46-2.30 (2H, m), 2.29 (3H, s), 2.17 (3H, s), 1.68 (4H, m), 1.49 (2H, m), 1.07 (6H, d). MASS (m/e) 539 [(M+H)⁺]

### EXAMPLE 15

### N-Isopropyl-N-[3-(piperidinomethyl)phenyl]-3-[3-(1-acenaphthenyl)-2-dicyanomethylidene imidazolidin-1-yl]propionamide (Compound 15)

Compound 15 (0.011 g, 20%) was obtained as a pale yellow oily substance using Compound M (0.040 g) obtained in Reference Example 13, 1-acenaphthenol (0.13 g), triphenylphosphine (0.21 g), diethyl azodicarboxylate (0.12 mL), and tetrahydrofuran (0.50 mL) as described in Example 14.

¹H NMR (270 MHz, CDCl₃) δ 7.77 (1H, brd), 7.67 (1H, brd), 7.60-7.22 (7H, m), 7.06 (1H, m), 6.39 (1H, m), 4.95 (1H, septet), 4.08-3.48 (7H, m), 3.38-2.94 (3H, m), 2.80-2.20 (6H, m), 1.71 (4H, m), 1.51 (2H, m), 1.07 (6H, d). MASS (m/e) 573 [(M+H)⁺]

### EXAMPLE 16

### N-Isopropyl-N-[3-(piperidinomethyl)phenyl]-3-[3-(1-naphthalenylmethyl)-2-dicyanomethylidene imidazolidin-1-yl]propionamide (Compound 16)

Compound 16 (0.12 g, 73%) was obtained as a pale yellow substance using Compound C (0.10 g) obtained in Reference Example 3, thionyl chloride (1.0 mL), Compound G (0.067 g) obtained in Reference Example 7, a 60% dispersion (0.023 g) of sodium hydride in mineral oil, and tetrahydrofuran (10.0 mL) as described in Example 3.

¹H NMR (270 MHz, CDCl₃) δ 7.91-7.84 (3H, m), 7.60-7.00 (8H, m), 5.18-5.17 (2H, m), 4.95 (1H, septet), 3.79-3.84 (2H, m), 3.63-3.57 (4H, m), 3.27-3.20(2H, m), 2.49-2.37 (6H, m), 1.60 (4H, m), 1.45 (2H, m), 1.06 (6H, d).

### EXAMPLE 17

### N-Isopropyl-N-[3-(piperidinomethyl)phenyl]-3-[3-(3,5-dichlorobenzyl)-2-dicyanomethylidene imidazolidin-1-yl]propionamide (Compound 17)

Compound 17 (0.13 g, 64%) was obtained as a pale yellow oily substance using Compound G (0.080 g) obtained in Reference Example 7, thionyl chloride (1.0 mL), Compound D (0.18 g) obtained in Reference Example 4, a 60% dispersion (0.029 g) of sodium hydride in mineral oil, and tetrahydrofuran (2.7 mL) as described in Example 3.

¹H NMR (270 MHz, CDCl₃) δ 7.43-6.99 (7H, m), 5.01-4.91 (1H, m), 4.75 (1H, d), 4.65 (1H, d), 3.83-3.76 (4H, m), 3.54-3.44 (4H, m), 2.40-2.36 (6H, m), 1.59-1.44 (6H, m), 1.07 (6H, d) MASS (m/e) 579 [(M+H)⁺]

### EXAMPLE 18

### N-Propyl-N-[2-methyl-5-(piperidinomethyl)phenyl]-3-[3-(3,5-dichlorobenzyl)-2-dicyanomethylidene imidazolidin-1-yl]propionamide (Compound 18)

Compound 18 (0.022 g, 18%) was obtained as a pale yellow oily substance using Compound O (0.12 g) obtained in Reference Example 15, 1-iodopropane (0.33 mL), potassium hydroxide (0.013 g), and dimethyl sulfoxide (0.42 mL) as described in Example 6.

¹H NMR (270 MHz, CDCl₃) δ 7.32 (1H, t), 7.28-7.24 (2H, m), 7.16 (2H, d), 7.13 (1H, brs), 4.77 (1H, d), 4.62 (1H, d), 4.03 (1H, m), 3.93-3.72 (4H, m), 3.57-3.38 (4H, m), 3.08 (1H, m), 2.50-2.20 (6H, m), 2.20 (3H, s), 1.69-1.38 (8H, m), 0.90 (3H, t). MASS (m/e) 593 [(M+H)⁺]

### EXAMPLE 19

### N-Propyl-N-[2-chloro-5-(piperidinomethyl)phenyl]-3-[3-(3,5-dichlorobenzyl)-2-dicyanomethylidene imidazolidin-1-yl]propionamide (Compound 19)

Compound 19 (0.034 g, 15%) was obtained as a pale yellow oily substance using Compound Q (0.22 g) obtained in Reference Example 17, 1-iodopropane (0.59 mL), potassium hydroxide (0.025 g), and dimethyl sulfoxide (0.76 mL) as described in Example 6.

¹H NMR (270 MHz, CDCl₃) δ 7.49 (1H, brd), 7.42-7.20 (3H, m), 7.18 (2H, d), 4.69 (2H, s), 3.96 (1H, m), 3.94-3.70 (4H, m), 3.58-3.32 (4H, m), 3.26 (1H, m), 2.45-2.24 (6H, m), 1.74-1.32 (8H, m), 0.91 (3H, t). MASS (m/e) 613 [(M+H)⁺]

### EXAMPLE 20

### N-Propyl-N-[2-methyl-3-(piperidinomethyl)phenyl]-3-[3-(3,5-dichlorobenzyl)-2-dicyanomethylidene imidazolidin-1-yl]propionamide (Compound 20)

Compound 20 (0.018 g, 14%) was obtained as a pale yellow oily substance using Compound S (0.11 g) obtained in Reference Example 19, 1-iodopropane (0.32 mL), potassium hydroxide (0.013 g), and dimethyl sulfoxide (0.41 mL) as described in Example 6.

¹H NMR (270 MHz, CDCl₃) δ 7.39 (1H, brd), 7.33 (1H, t), 7.24 (1H, t), 7.17 (2H, d), 6.99 (1H, brd), 4.70 (2H, s), 4.02 (1H, m), 3.89-3.73 (4H, m), 3.51-3.37 (4H, m), 3.09 (1H, m), 2.50-2.27 (6H, m), 2.22 (3H, s), 1.67-1.37 (8H, m), 0.90 (3H, t). MASS (m/e) 593 [(M+H)⁺]

### EXAMPLE 21

### N-Ethyl-N-[3-(propylaminomethyl)phenyl]-3-[3-(3,5-dimethylbenzyl)-2-dicyanomethylidene imidazolidin-1-yl]propionamide (Compound 21)

Trifluoroacetic acid (1.5 mL) was added to Compound T (0.15 g) obtained in Reference Example 20. After 10 minutes, the solvent was evaporated under reduced pressure, and a saturated aqueous sodium bicarbonate solution was added thereto, followed by extraction with ethyl acetate. The extract was washed with a saturated aqueous sodium chloride solution and dried over anhydrous magnesium sulfate. The solvent was evaporated under reduced pressure. The residue was purified by silica gel column chromatography (chloroform:methanol=10:1) to give Compound 21 (0.10 g, 83%) as a pale yellow oily substance.

¹H NMR (270 MHz, CDCl₃) δ 7.46-7.35 (2H, m), 7.30-7.18 (1H, m), 7.12-7.01 (1H, m), 6.95 (1H, s), 6.86 (2H, s), 4.63 (2H, s), 3.92 (2H, s), 3.90-3.60 (6H, m), 3.40 (2H, dd), 2.74 (2H, t), 2.46 (2H, t), 2.31 (6H, s), 1.62 (2H, tq), 1.12 (3H, t), 0.96 (3H, t). The signal which corresponds to a secondary amino group was not observed. MASS (m/e) 499 [(M+H)⁺]

### EXAMPLE 22

### N-Isopropyl-N-[3-(morpholinomethyl)phenyl]-3-[3-(3,5-dimethylbenzyl)-2-dicyanomethylidene imidazolidin-1-yl]propionamide (Compound 22)

Compound W (0.058 g) obtained in Reference Example 22, sodium iodide (0.018 g), and morpholine (0.10 mL) were dissolved in acetonitrile (10 mL), followed by stirring at room temperature for 12 hours. The solvent was removed by evaporation, an aqueous sodium bicarbonate solution was added thereto, followed by extraction with ethyl acetate, the extract was dried over anhydrous sodium sulfate, and the solvent was evaporated. The residue was purified by silica gel thin layer chromatography (chloroform:methanol=10:1) to give Compound 22 (0.044 g, 69%) as a pale yellow oily substance.

¹H NMR (270 MHz, CDCl₃) δ 7.44-7.36 (2H, m), 7.12 (1H, brs), 7.02-6.93 (1H, m), 6.89 (1H, brs), 6.84 (2H, brs), 4.93 (1H, septet), 4.66-4.58 (2H, m), 3.79-3.74 (2H, m), 3.73-3.66 (6H, m), 3.55-3.54 (2H, m), 3.40-3.33 (2H, m), 2.46-2.42 (4H, m), 2.35 (2H, t), 2.29 (6H, s), 1.04 (6H, d). MASS (m/e) 541 [(M+H)⁺]

### EXAMPLE 23

### N-Isopropyl-N-[3-(4-methyl-1-piperazinylmethyl)phenyl]-3-[3-(3,5-dimethylbenzyl)-2-dicyanomethylidene imidazolidin-1-yl]propionamide (Compound 23)

Compound 23 (0.039 g, 68%) was obtained as a pale yellow oily substance using Compound W (0.050 g) obtained in Reference Example 22, sodium iodide (0.015 g), 1-methylpiperazine (0.11 mL), and acetonitrile (10 mL) as described in Example 22.

¹H NMR (270 MHz, CDCl₃) δ 7.42-7.34 (2H, m), 7.11 (1H, brs), 6.99-6.96 (1H, m), 6.93 (1H, brs), 6.84 (2H, brs), 4.93 (1H, septet), 4.67-4.58 (2H, m), 3.79-3.74 (2H, m), 3.72-3.66 (2H, m), 3.56 (2H, s), 3.47 (3H, s), 3.40-3.33 (2H, m), 2.47 (8H, m), 2.35 (2H, m), 2.29 (6H, s), 1.04 (6H, d). MASS (m/e) 554 [(M+H)⁺]

### EXAMPLE 24

### N-Isopropyl-N-[3-(diethylaminomethyl)phenyl]-3-[3-(3,5-dimethylbenzyl)-2-dicyanomethylidene imidazolidin-1-yl]propionamide (Compound 24)

Compound 24 (0.034 g, 64%) was obtained as a pale yellow oily substance using Compound W (0.050 g) obtained in Reference Example 22, sodium iodide (0.015 g), diethylamine (0.11 mL), and acetonitrile (10 mL) as described in Example 22.

¹H NMR (270 MHz, CDCl₃) δ 7.39-7.37 (2H, m), 7.12 (1H, brs), 6.96 (1H, m), 6.93 (1H, brs), 6.84 (2H, brs), 4.93 (1H, septet), 4.66-4.58 (2H, m), 3.80-3.76 (2H, m), 3.71-3.65 (2H, m), 3.61 (2H, s), 3.40-3.33 (2H, m), 2.52 (4H, q), 2.35 (2H, m), 2.29 (6H, s), 1.05-1.00 (12H, m). MASS (m/e) 527 [(M+H)⁺]

### EXAMPLE 25

### N-Isopropyl-N-[3-(4-ethoxycarbonylpiperidinomethyl)phenyl]-3-[3-(3,5-dimethylbenzyl)-2-dicyanomethylidene imidazolidin-1-yl]propionamide (Compound 25)

Compound 25 (0.059 g, 96%) was obtained as a pale yellow oily substance using Compound W (0.050 g) obtained in Reference Example 22, sodium iodide (0.015 g), ethyl isonipecotate (0.16 g) and acetonitrile (10 mL) as described in Example 22.

¹H NMR (270 MHz, CDCl₃) δ 7.39-7.36 (2H, m), 7.11 (1H, brs), 6.98-6.95 (1H, m), 6.93 (1H, brs), 6.84 (2H, brs), 4.93 (1H, septet), 4.67-4.57 (2H, m), 4.11 (2H, q), 3.80-3.75 (2H, m), 3.72-3.65 (2H, m), 3.54-3.52 (2H, m), 3.40-3.33 (2H, m), 2.85-2.80 (2H, m), 2.37-2.23 (9H, m), 2.06-2.02 (2H, m), 1.90-1.75 (4H, m), 1.23 (3H, t), 1.04 (6H, d). MASS (m/e) 611 [(M+H)⁺]

### EXAMPLE 26

### N-Isopropyl-N-[3-(bis(2-hydroxyethyl)aminomethyl)phenyl]-3-[3-(3,5-dimethylbenzyl)-2-dicyanomethylidene imidazolidin-1-yl]propionamide (Compound 26)

Compound 26 (0.053 g, 93%) was obtained as a pale yellow oily substance using Compound W (0.050 g) obtained in Reference Example 22, sodium iodide (0.015 g), diethanolamine (0.098 mL) and acetonitrile (10 mL) as described in Example 22.

¹H NMR (270 MHz, CDCl₃) δ 7.42-7.32 (2H, m), 7.22 (1H, brs), 6.98-6.95 (1H, m), 6.93 (1H, brs), 6.83 (2H, brs), 4.93 (1H, septet), 4.64-4.57 (2H, m), 3.76-3.65 (6H, m), 3.58 (4H, t), 3.44-3.32 (2H, m), 2.70 (4H, t), 2.42-2.39 (2H, m), 2.31 (6H, s), 1.05 (6H, d). The signals which correspond to two hydroxyl groups were not observed. MASS (m/e) 559 [(M+H)⁺]

### EXAMPLE 27

### N-Isopropyl-N-[3-[(N-(2-hydroxyethyl)-N-methylamino)methyl]phenyl]-3-[3-(3,5-dimethylbenzyl)-2-dicyanomethylidene imidazolidin-1-yl]propionamide (Compound 27)

Compound 27 (0.038 g, 75%) was obtained as a pale yellow oily substance using Compound W (0.047 g) obtained in Reference Example 22, sodium iodide (0.015 g), N-methylethanolamine (0.072 g) and acetonitrile (10 mL) as described in Example 22.

¹H NMR (270 MHz, CDCl₃) δ 7.46-7.35 (2H, m), 7.15 (1H, brs), 7.03-7.00 (1H, m), 6.95 (1H, brs), 6.86 (2H, brs), 4.95 (1H, septet), 4.68-4.58 (2H, m), 3.81-3.76 (2H, m), 3.75-3.60 (6H, m), 3.39-3.35 (2H, m), 2.67 (2H, t), 2.38 (2H, t), 2.31 (6H, s), 2.30 (3H, s), 1.07 (6H, d). The signal which corresponds to a hydroxyl group was not observed.

### EXAMPLE 28

### N-(3-Methoxycarbonylbenzyl)-N-[3-(piperidinomethyl)phenyl]-3-[3-(3,5-dimethylbenzyl)-2-dicyanomethylidene imidazolidin-1-yl]propionamide (Compound 28)

Compound 28 (0.48 g, 73%) was obtained as a pale yellow oily substance using Compound B (0.33 g) obtained in Reference Example 2, Compound X (0.35 g) obtained in Reference Example 23, thionyl chloride (3.0 mL), a 60% dispersion (0.065 g) of sodium hydride in mineral oil, and tetrahydrofuran (6.0 mL) as described in Example 3.

¹H NMR (270 MHz, CDCl₃) δ 7.91 (1H, m), 7.84 (1H, m), 7.43-7.24 (4H, m), 7.01 (1H, brs), 6.95 (1H, brs), 6.91 (1H, m), 6.86 (2H, brs), 4.93 (2H, brs), 4.64 (2H, s), 3.94-3.78 (2H, m), 3.87 (3H, s), 3.76-3.62 (2H, m), 3.47 (2H, brs), 3.46-3.32 (2H, m), 2.64-2.46 (2H, m), 2.32 (4H, m), 2.30 (6H, s), 1.56 (4H, m), 1.43 (2H, m). MASS (m/e) 644 [(M+H)⁺]

### EXAMPLE 29

### N-(3-Carboxybenzyl)-N-[3-(piperidinomethyl)phenyl]-3-[3-(3,5-dimethylbenzyl)-2-dicyanomethylidene imidazolidin-1-yl]propionamide (Compound 29)

Compound 28 (0.48 g) obtained in Example 28 was dissolved in tetrahydrofuran (8.0 mL), and a 1.4 mol/L aqueous lithium hydroxide solution (8.0 mL) and methanol (8.0 mL) were added thereto, followed by stirring at room temperature for 1 hour. The solvent was evaporated under reduced pressure, and water, 1 mol/L hydrochloric acid and a saturated aqueous sodium bicarbonate solution were added thereto to adjust the pH to about 8, followed by extraction with chloroform. The extract was dried over anhydrous sodium sulfate, and the solvent was evaporated under reduced pressure. The residue was purified by silica gel preparative thin layer chromatography (chloroform:methanol=5:1) to give Compound 29 (0.28 g, 59%) as colorless crystals.

¹H NMR (270 MHz, CD₃OD) δ 7.90-7.74 (2H, m), 7.52-7.36 (2H, m), 7.36-7.16 (4H, m), 6.91 (1H, brs), 6.87 (2H, brs), 4.95 (2H, brs), 4.59 (2H, brs), 4.09 (2H, brs), 3.90-3.32 (6H, m), 2.87 (4H, m), 2.68-2.42 (2H, m), 2.24 (6H, s), 1.73 (4H, m), 1.52 (2H, m). The signal which corresponds to a carboxyl group was not observed. MASS (m/e) 631 [(M+H)+]

### EXAMPLE 30

### N-(3-Methoxycarbonyl-α-methylbenzyl)-N-[3-(piperidinomethyl)phenyl]-3-[3-(3,5-dimethylbenzyl)-2-dicyanomethylidene imidazolidin-1-yl]propionamide

Compound 30 (0.22 g, 41%) was obtained as a pale yellow oily substance using Compound B (0.29 g) obtained in Reference Example 2, Compound Y (0.29 g) obtained in Reference Example 24, thionyl chloride (2.5 mL), a 60% dispersion (0.067 g) of sodium hydride in mineral oil, and tetrahydrofuran (5.0 mL) as described in Example 3.

¹H NMR (270 MHz, CDCl₃) δ 8.04-7.76 (2H, m), 7.50-6.04 (10H, m), 4.82-4.46 (2H, m), 4.10-3.04 (11H, m), 2.76-1.10 (21H, m). MASS (m/e) 659 [(M+H)⁺]

### EXAMPLE 31

### N-(3-Carboxy-α-methylbenzyl)-N-[3-(piperidinomethyl)phenyl]-3-[3-(3,5-dimethylbenzyl)-2-dicyanomethylidene imidazolidin-1-yl]propionamide (Compound 31)

Compound 31 (0.13 g, 60%) was obtained as a pale yellow amorphous solid using Compound 30 (0.22 g) obtained in Example 30, a 1.3 mol/L aqueous lithium hydroxide solution (7.0 mL), tetrahydrofuran (7.0 mL), and methanol (7.0 mL) as described in Example 29.

¹H NMR (270 MHz, CD₃OD) δ 8.00-6.00 (12H, m), 4.72-4.42 (2H, m), 4.38-2.04 (20H, m), 2.00-1.24 (9H, m). The signal which corresponds to a carboxyl group was not observed. MASS (m/e) 645 [(M+H)⁺]

### EXAMPLE 32

### N-(5-Methoxycarbonylfurfuryl)-N-[3-(piperidinomethyl)phenyl]-3-[3-(3,5-dimethylbenzyl)-2-dicyanomethylidene imidazolidin-1-yl]propionamide (Compound 32)

Compound 32 (0.10 g, 43%) was obtained as a pale yellow oily substance using Compound B (0.15 g) obtained in Reference Example 2, Compound Z (0.12 g) obtained in Reference Example 25, thionyl chloride (0.80 mL), a 60% dispersion (0.032 g) of sodium hydride in mineral oil, and tetrahydrofuran (2.4 mL) as described in Example 3.

¹H NMR (270 MHz, CDCl₃) δ 7.43-7.28 (2H, m), 7.13 (1H, brs), 7.05 (1H, d), 6.99 (1H, m), 6.95 (1H, brs), 6.86 (2H, brs), 6.29 (1H, d), 4.92 (2H, s), 4.65 (2H, s), 3.94-3.77 (2H, m), 3.83 (3H, s), 3.77-3.63 (2H, m), 3.50 (2H, brs), 3.48-3.36 (2H, m), 2.60-2.48 (2H, m), 2.37 (4H, m), 2.30 (6H, s), 1.58 (4H, m), 1.45 (2H, m). MASS (m/e) 635 [(M+H)⁺]

### EXAMPLE 33

### N-(5-Carboxyfurfuryl)-N-[3-(piperidinomethyl)phenyl]-3-[3-(3,5-dimethylbenzyl)-2-dicyanomethylidene imidazolidin-1-yl]propionamide (Compound 33)

Compound 33 (0.049 g, 83%) was obtained as a pale yellow amorphous solid using Compound 32 (0.060 g) obtained in Example 32, a 1.6 mol/L aqueous lithium hydroxide solution (1.0 mL), tetrahydrofuran (1.0 mL), and methanol (1.0 mL) as described in Example 29.

¹H NMR (270 MHz, CD₃OD) δ 7.54-7.40 (3H, m), 7.34 (1H, m), 6.93 (1H, brs), 6.89 (2H, brs), 6.78 (1H, d), 6.22 (1H, brd), 4.90 (2H, brs), 4.61 (2H, s), 4.20 (2H, s), 3.84-3.70 (2H, m), 3.70-3.55 (2H, m), 3.53-3.38 (2H, m), 3.03 (4H, m), 2.60-2.46 (2H, m), 2.26 (6H, s), 1.80 (4H, m), 1.60 (2H, m). The signal which corresponds to a carboxyl group was not observed. MASS (m/e) 621 [(M+H)⁺]

### EXAMPLE 34

### N-(5-Methoxycarbonyl-2-thenyl)-N-[3-(piperidinomethyl)phenyl]-3-[3-(3,5-dimethylbenzyl)-2-dicyanomethylidene imidazolidin-1-yl]propionamide (Compound 34)

Compound 34 (0.26 g, 73%) was obtained as a pale yellow oily substance using Compound B (0.21 g) obtained in Reference Example 2, Compound AA (0.19 g) obtained in Reference Example 26, thionyl chloride (1.0 mL), a 60% dispersion (0.039 g) of sodium hydride in mineral oil, and tetrahydrofuran (4.0 mL) as described in Example 3.

¹H NMR (270 MHz, CDCl₃) δ 7.57 (1H, d), 7.42-7.32 (2H, m), 7.09 (1H, brs), 6.97 (1H, m), 6.95 (1H, brs), 6.86 (2H, brs), 6.81 (1H, brd), 4.99 (2H, brs), 4.64 (2H, s), 3.90-3.78 (2H, m), 3.83 (3H, s), 3.77-3.63 (2H, m), 3.50 (2H, brs), 3.47-3.35 (2H, m), 2.60-2.48 (2H, m), 2.37 (4H, m), 2.30 (6H, s), 1.57 (4H, m), 1.44 (2H, m). MASS (m/e) 651 [(M+H)⁺]

### EXAMPLE 35

### N-(5-Carboxy-2-thenyl)-N-[3-(piperidinomethyl)phenyl]-3-[3-(3,5-dimethylbenzyl)-2-dicyanomethylidene imidazolidin-1-yl]propionamide (Compound 35)

Compound 35 (0.16 g, 67%) was obtained as a pale yellow amorphous solid using Compound 34 (0.24 g) obtained in Example 34, a 1.4 mol/L aqueous lithium hydroxide solution (4.0 mL), tetrahydrofuran (4.0 mL), and methanol (4.0 mL) as described in Example 29.

¹H NMR (270 MHz, CDCl₃) δ 8.15 (1H, brs), 7.52-7.22 (4H, m), 7.13 (1H, m), 6.92 (1H, brs), 6.85 (2H, brs), 6.67 (1H, brd), 4.92 (2H, brs), 4.62 (2H, brs), 4.05 (2H, brs), 3.94-3.54 (4H, m), 3.54-3.26 (2H, m), 2.88 (4H, m), 2.64-2.38 (2H, m), 2.28 (6H, s), 1.82 (4H, m), 1.52 (2H, m). MASS (m/e) 637 [(M+H)⁺]

### EXAMPLE 36

### N-(4-Hydroxy-3-nitrobenzyl)-N-[3-(piperidinomethyl)phenyl]-3-[3-(3,5-dimethylbenzyl)-2-dicyanomethylidene imidazolidin-1-yl]propionamide (Compound 36)

Compound 36 (0.16 g, 35%) was obtained as a yellow oily substance using Compound B (0.47 g) obtained in Reference Example 2, Compound AB (0.25 g) obtained in Reference Example 27, thionyl chloride (2.5 mL), a 60% dispersion (0.20 g) of sodium hydride in mineral oil, and tetrahydrofuran (8.0 mL) as described in Example 3.

¹H NMR (270 MHz, CDCl₃) δ 7.86 (1H, d), 7.64 (1H, brs), 7.46 (1H, dd), 7.40-7.25 (2H, m), 7.10-7.02 (2H, m), 6.98-6.87 (2H, m), 6.86 (2H, brs), 4.85 (2H, brs), 4.63 (2H, s), 3.92-3.76 (2H, m), 3.74-3.60 (2H, m), 3.51 (2H, brs), 3.48-3.33 (2H, m), 2.60-2.46 (2H, m), 2.36 (4H, m), 2.29 (6H, s), 1.56 (4H, m), 1.44 (2H, m). MASS (m/e) 648 [(M+H)⁺]

### EXAMPLE 37

### N-[4-(1H-Tetrazol-5-yl)benzyl]-N-[3-(piperidinomethyl)phenyl]-3-[3-(3,5-dimethylbenzyl)-2-dicyanomethylidene imidazolidin-1-yl]propionamide (Compound 37)

Compound AF (0.45 g) obtained in Reference Example 31 was dissolved in methanol (3.0 mL) and chloroform (3.0 mL), 1 mol/L hydrochloric acid (1.0 mL) was added thereto, followed by stirring at room temperature for 40 minutes, and 6 mol/L hydrochloric acid (1.0 mL) and methanol (2.0 mL) were further added thereto, followed by stirring at room temperature for 1.5 hours. The solvent was evaporated under reduced pressure, and water and a saturated aqueous sodium bicarbonate solution were added thereto to adjust the pH to about 8, followed by extraction with chloroform. The extract was dried over anhydrous sodium sulfate, and the solvent was evaporated under reduced pressure. The residue was purified by silica gel preparative thin layer chromatography (chloroform:methanol=5:1) to give Compound 37 (0.23 g, 74%) as a pale yellow amorphous solid.

¹H NMR (270 MHz, CD₃OD) δ 7.92 (2H, m), 7.52-7.17 (5H, m), 7.13 (1H, brs), 6.90 (1H, brs), 6.86 (2H, brs), 4.97 (2H, brs), 4.59 (2H, s), 3.93 (2H, brs), 3.92-3.72 (2H, m), 3.72-3.52 (2H, m), 3.52-3.34 (2H, m), 2.70 (4H, m), 2.66-2.44 (2H, m), 2.24 (6H, s), 1.57 (4H, m), 1.39 (2H, m). The signal which corresponds to a tetrazolyl group was not observed. MASS (m/e) 655 [(M+H)⁺]

### EXAMPLE 38

### N-[α-Methyl-3-(1H-tetrazol-5-yl)benzyl]-N-[3-(piperidinomethyl)phenyl]-3-[3-(3,5-dimethylbenzyl)-2-dicyanomethylidene imidazolidin-1-yl]propionamide (Compound 38)

Compound AJ (0.052 g) obtained in Reference Example 35 was dissolved in methanol (1.0 mL) and chloroform (0.5 mL), and 6 mol/L hydrochloric acid (0.20 mL) was added thereto, followed by stirring at room temperature for 1.5 hours. The solvent was evaporated under reduced pressure, and water and a saturated aqueous sodium bicarbonate solution was added thereto to adjust the pH to about 8, followed by extraction with chloroform. The extract was dried over anhydrous sodium sulfate, and the solvent was evaporated under reduced pressure. The residue was purified by silica gel preparative thin layer chromatography (chloroform:methanol=5:1) to give Compound 38 (0.033 g, 88%) as a pale yellow amorphous solid.

¹H NMR (270 MHz, CD₃OD) δ 8.06-6.00 (12H, m), 4.74-4.45 (2H, m), 4.45-2.05 (20H, m), 2.00-1.10 (9H, m). The signal which corresponds to a tetrazolyl group was not observed. MASS (m/e) 669 [(M+H)⁺]

### EXAMPLE 39

### N-(1H-Benzotriazol-5-ylmethyl)-N-[3-(piperidinomethyl)phenyl]-3-[3-(3,5-dimethylbenzyl)-2-dicyanomethylidene imidazolidin-1-yl]propionamide (Compound 39)

Compound AL (0.15 g) obtained in Reference Example 37 was dissolved in tetrahydrofuran (5.0 mL), and 6 mol/L hydrochloric acid (5.0 mL) was added thereto, followed by stirring at room temperature for 1 hour and further stirring at 50° C. for 1 hour. The mixture was allowed to stand for cooling until room temperature, and then a saturated aqueous sodium bicarbonate solution was added thereto to adjust the pH to about 8, followed by extraction with chloroform. The extract was dried over anhydrous sodium sulfate, and the solvent was evaporated under reduced pressure. The residue was purified by silica gel preparative thin layer chromatography (chloroform:methanol=10:1) to give Compound 39 (0.093 g, 72%) as a pale yellow oily substance.

¹H NMR (270 MHz, CDCl₃) δ 9.86 (1H, brs), 7.68 (1H, d), 7.59 (1H, brs), 7.32-7.17 (3H, m), 7.10 (1H, brs), 6.98 (1H, m), 6.91 (1H, brs), 6.84 (2H, brs), 5.01 (2H, brs), 4.62 (2H, s), 3.94-3.80 (2H, m), 3.72-3.58 (2H, m), 3.52 (2H, brs), 3.46-3.32 (2H, m), 2.64-2.50 (2H, m), 2.37 (4H, m), 2.26 (6H, s), 1.50 (4H, m), 1.39 (2H, m). MASS (m/e) 628 [(M+H)⁺]

### EXAMPLE 40

### N-Isopropyl-N-[3-(piperidinomethyl)phenyl]-3-[3-(2,3,5-trimethylbenzyl)-2-dicyanomethylidene imidazolidin-1-yl]propionamide (Compound 40)

Compound 40 (0.031 g, 54%) was obtained as a pale yellow oily substance using Compound M (0.043 g) obtained in Reference Example 13, Compound AO (0.012 g) obtained in Reference Example 40, triphenylphosphine (0.22 g), diethyl azodicarboxylate (0.13 mL) and tetrahydrofuran (0.50 mL) as described in Example 14.

¹H NMR (270 MHz, CDCl₃) δ7.50-7.30 (2H, m), 7.13 (1H, brs), 7.06-6.86 (2H, m), 6.78 (1H, brs), 4.95 (1H, septet), 4.80-4.58 (2H, m), 3.90-3.40 (6H, m), 3.38-3.14 (2H, m), 2.56-2.20 (6H, m), 2.27 (3H, brs), 2.25 (3H, brs), 2.12 (3H, brs), 1.58 (4H, m), 1.45 (2H, m), 1.06 (6H, d). MASS (m/e) 553 [(M+H)⁺]

### EXAMPLE 41

### N-[(1-Isopropyl-3-methoxycarbonylpyrazol-5-yl)methyl]-N-[3-(piperidinomethyl)phenyl]-3-[3-(3,5-dimethylbenzyl)-2-dicyanomethylidene imidazolidin-1-yl]propionamide (Compound 41)

Thionyl chloride (4.0 mL) was added to Compound B (0.39 g) obtained in Reference Example 2, followed by stirring at room temperature for 10 minutes. Thionyl chloride was evaporated under reduced pressure. Toluene was added to the residue to cause azeotropy to give a crude acid chloride. Separately, Compound AS (0.36 g) obtained in Reference Example 44 was dissolved in toluene (4.0 mL) and N,N-dimethylformamide (0.40 mL). To the resulting mixture was added dropwise a solution of the above prepared acid chloride in toluene-N,N-dimethylformamide (10:1; 2.2 mL), followed by stirring at room temperature for 1 hour. A saturated aqueous sodium bicarbonate solution and water were added thereto to adjust the pH to about 8, followed by extraction with ethyl acetate. The extract was dried over anhydrous sodium sulfate, and the solvent was evaporated under reduced pressure. The residue was purified by silica gel column chromatography (chloroform:methanol=8:1) to give Compound 41 (0.53 g, 80%) as a pale yellow oily substance.

¹H NMR (270 MHz, CDCl₃) δ 7.43-7.26 (2H, m), 7.04 (1H, brs), 6.95 (1H, brs), 6.88 (1H, m), 6.85 (2H, brs), 6.46 (1H, s), 4.97 (2H, brs), 4.64 (2H, brs), 4.56 (1H, septet), 3.86 (3H, s), 3.94-3.74 (2H, m), 3.74-3.56 (2H, m), 3.48-3.30 (2H, m), 3.47 (2H, brs), 2.60-2.42 (2H, m), 2.34 (4H, m), 2.30 (6H, s), 1.56 (4H, m), 1.44 (2H, m), 1.42 (6H, d). MASS (m/e) 677 [(M+H)⁺]

### EXAMPLE 42

### N-[(3-Carboxy-1-isopropylpyrazol-5-yl)methyl]-N-[3-(piperidinomethyl)phenyl]-3-[3-(3,5-dimethylbenzyl)-2-dicyanomethylidene imidazolidin-1-yl]propionamide (Compound 42)

Compound 42 (0.13 g, 25%) was obtained as a pale yellow oily amorphous solid using Compound 41 (0.53 g) obtained in Example 41, a 2.1 mol/L aqueous lithium hydroxide solution (5.0 mL), tetrahydrofuran (5.0 mL) and methanol (5.0 mL) as described in Example 29.

¹H NMR (270 MHz, CD₃OD) δ 7.60-7.40 (2H, m), 7.37-7.16 (2H, m), 6.94 (1H, brs), 6.89 (2H, brs), 6.33 (1H, brs), 5.06 (2H, brs), 4.66 (1H, m), 4.62 (2H, brs), 4.05 (2H, brs), 3.90-3.34 (6H, m), 2.88 (4H, m), 2.68-2.40 (2H, m), 2.27 (6H, s), 1.76 (4H, m), 1.57 (2H, m), 1.36 (6H, brd). The signal which corresponds to a carboxyl group was not observed. MASS (m/e) 663 [(M+H)⁺]

### EXAMPLE 43

### N-[5-(Methanesulfonylaminocarbonyl)furfuryl]-N-[3-(piperidinomethyl)phenyl]-3-[3-(3,5-dimethylbenzyl)-2-dicyanomethylidene imidazolidin-1-yl]propionamide (Compound 43)

Compound 33 (0.017 g) obtained in Example 33 was dissolved in dichloromethane (0.1 mL), and 1-(3-dimethylaminopropyl)-3-ethylcarbodiimide hydrochloride (0.0069 g), 4-dimethylaminopyridine (0.0016 g), and methanesulfonyl amine (0.0059 g) were added thereto, followed by stirring at room temperature for 2 hours. Water was added thereto, followed by extraction with chloroform-isopropanol (4:1). The extract was dried over anhydrous sodium sulfate, and the solvent was evaporated under reduced pressure. The residue was purified by silica gel column chromatography (chloroform:methanol=3:2) to give Compound 43 (0.0045 g, 24%) as a pale yellow oily substance.

¹H NMR (270 MHz, CDCl₃) δ 7.60 (1H, brs), 7.48-7.32 (2H, m), 7.16 (1H, m), 6.94 (1H, brs), 6.87 (1H, d), 6.85 (2H, brs), 6.15 (1H, d), 4.80 (2H, brs), 4.63 (2H, s), 4.11 (2H, brs), 3.90-3.50 (4H, m), 3.48-3.24 (2H, m), 3.06 (3H, s), 2.99 (4H, m), 2.64-2.40 (2H, m), 2.29 (6H, s), 1.86 (4H, m), 1.26 (2H, m). The signal which corresponds to a sulfonamido group was not observed. MASS (m/e) 698 [(M+H)⁺]

### EXAMPLE 44

### N-[(4-Methoxycarbonylquinolin-2-yl)methyl]-N-[3-(piperidinomethyl)phenyl]-3-[3-(3,5-dimethylbenzyl)-2-dicyanomethylidene imidazolidin-1-yl]propionamide (Compound 44)

Compound 44 (0.50 g, 78%) was obtained as a pale yellow oily substance using Compound B (0.33 g) obtained in Reference Example 2, Compound AT (0.36 g) obtained in Reference Example 45, thionyl chloride (4.0 mL), and toluene-N,N-dimethylformamide (10:1; 6.6 mL) as described in Example 41.

¹H NMR (270 MHz, CDCl₃) δ 8.70 (1H, m), 8.00 (1H, m), 7.95 (1H, s), 7.68 (1H, m), 7.60 (1H, m), 7.42-7.10 (4H, m), 6.95 (1H, brs), 6.85 (2H, brs), 5.21 (2H, brs), 4.63 (2H, brs), 4.03 (3H, s), 3.96-3.82 (2H, m), 3.72-3.57 (2H, m), 3.46 (2H, brs), 3.38-3.22 (2H, m), 2.76-2.58 (2H, m), 2.30 (6H, s), 2.29 (4H, m), 1.47 (4H, m), 1.38 (2H, m). MASS (m/e) 696 [(M+H)⁺]

### EXAMPLE 45

### N-[(4-Carboxyquinolin-2-yl)methyl]-N-[3-(piperidinomethyl)phenyl]-3-[3-(3,5-dimethylbenzyl)-2-dicyanomethylidene imidazolidin-1-yl]propionamide (Compound 45)

Compound 45 (0.16 g, 33%) was obtained as a pale yellow amorphous solid using Compound 44 (0.50 g) obtained in Example 44, a 2.1 mol/L aqueous lithium hydroxide solution (5.0 mL), tetrahydrofuran (5.0 mL) and methanol (5.0 mL) as described in Example 29.

¹H NMR (270 MHz, CD₃OD) δ 8.40 (1H, m), 7.87 (1H, m), 7.66 (1H, s), 7.65 (1H, m), 7.53 (1H, m), 7.48-7.22 (4H, m), 6.93 (1H, brs), 6.89 (2H, brs), 5.22 (2H, brs), 4.61 (2H, brs), 4.00-3.52 (6H, m), 3.52-3.32 (2H, m), 2.76-2.30 (6H, m), 2.26 (6H, s), 1.52 (4H, m), 1.40 (2H, m). The signal which corresponds to a carboxyl group was not observed. MASS (m/e) 682 [(M+H)⁺]

The chemical formulae of Compounds 1 to 45 are shown in Tables 1-6 below.

In Tables 1-6, Me means a methyl group; Et means an ethyl group; ⁿPr means a n-propyl group; ⁱPr means an isopropyl group; and Ph means a phenyl group.

TABLE 1

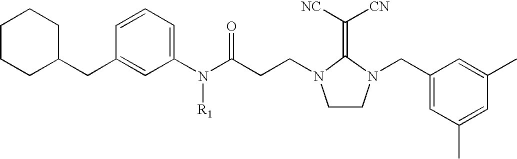

| Compound Number | R₁ |
|---|---|
| 1 | Me |
| 2 | ⁿPr |
| 3 | ⁱPr |
| 4 | cyclohexyl |
| 5 | CH₂Ph |
| 6 | 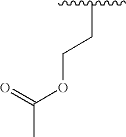 |
| 7 | 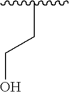 |
| 8 | 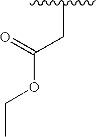 |
| 9 | 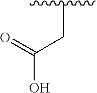 |
| 10 | 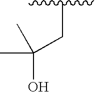 |
| 11 | 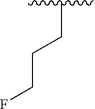 |
| 12 | 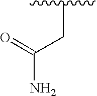 |
| 13 | 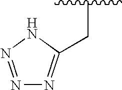 |

TABLE 2

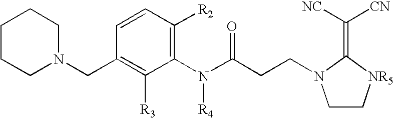

| Compound Number | R₂ | R₃ | R₄ | R₅ |
|---|---|---|---|---|
| 14 | H | H | ⁱPr | 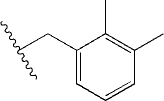 |
| 15 | H | H | ⁱPr | 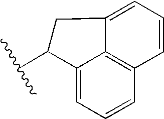 |
| 16 | H | H | ⁱPr | 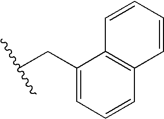 |
| 17 | H | H | ⁱPr | 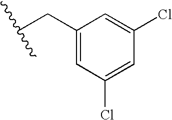 |
| 18 | Me | H | ⁿPr | 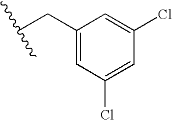 |
| 19 | Cl | H | ⁿPr | 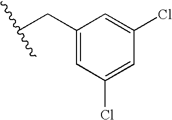 |
| 20 | H | Me | ⁿPr | 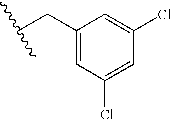 |
| 40 | H | H | ⁱPr | 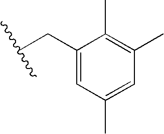 |

TABLE 3

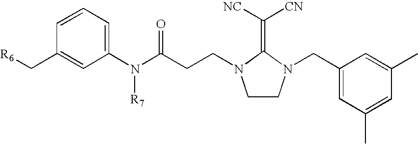

| Compound Number | R₆ | R₇ |
|---|---|---|
| 21 | 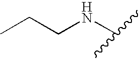 | Et |
| 22 | 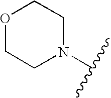 | ⁱPr |
| 23 | 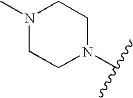 | ⁱPr |
| 24 | 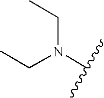 | ⁱPr |
| 25 | 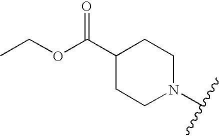 | ⁱPr |
| 26 | 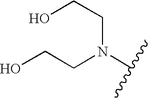 | ⁱPr |
| 27 | 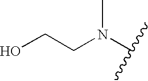 | ⁱPr |

Compounds 28 to 32 shown in Table 4 were synthesized in the above-described process.

TABLE 4

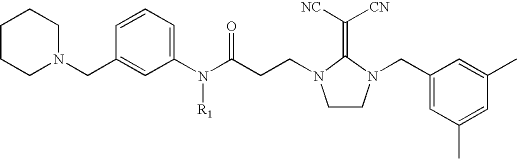

| Compound Number | R₁ |
|---|---|
| 28 | 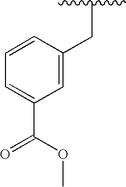 |
| 29 | 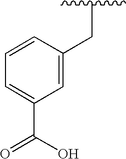 |
| 30 | 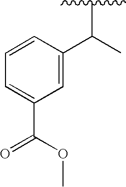 |
| 31 | 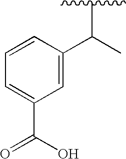 |
| 32 | 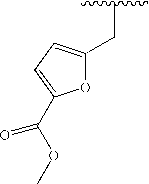 |
| 33 | 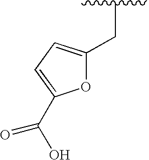 |

TABLE 5

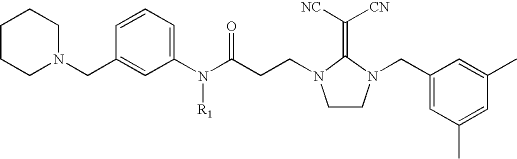

| Compound Number | R₁ |
|---|---|
| 34 | 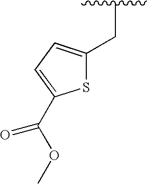 |
| 35 | 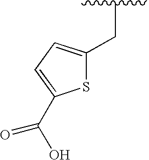 |
| 36 | 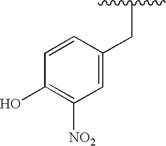 |
| 37 | 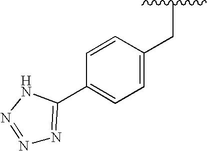 |
| 38 | 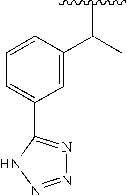 |
| 39 | 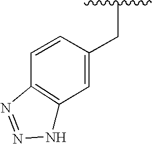 |

TABLE 6

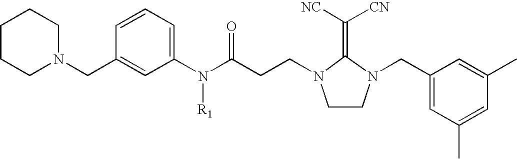

| Compound Number | R₁ |
|---|---|
| 41 | 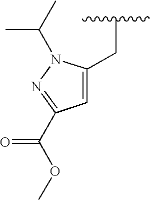 |
| 42 | 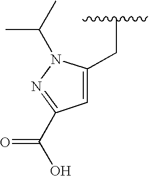 |
| 43 | 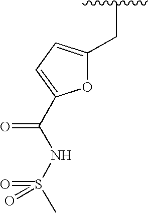 |
| 44 | 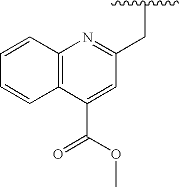 |
| 45 | 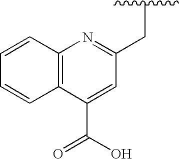 |

### REFERENCE EXAMPLE 1

### 2-[1-(2-Ethoxycarbonylethyl)-imidazolidinylidene]propanedinitrile (Compound A)

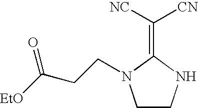

Step 1:

[(2-Ethoxycarbonylethylamino)(2-hydroxyethylamino)methylidene]propanedinitrile (Compound Aa):

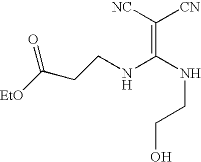

A mixture of β-alanine ethyl ester hydrochloride (30 g), triethylamine (38 mL), [bis(methylthio)methylidene]propanedinitrile (32 g) and ethanol (310 mL) was stirred at room temperature for 1 hour. The solvent was evaporated under reduced pressure, and 2-aminoethanol (20 mL) was added thereto, followed by stirring at 70° C. for 2 hours. After cooling, the reaction mixture was purified by silica gel column chromatography (chloroform:methanol=30:1 to 20:1) to give Compound Aa (28 g, 60%) as a pale yellow oily substance.

¹H NMR (270 MHz, CDCl₃) δ 7.00-6.78 (1H, m), 6.30-6.12 (1H, m), 4.19 (2H, q), 3.91-3.60 (4H, m), 3.57-3.30 (2H, m), 2.80-2.55 (3H, m), 1.29 (3H, t).

Step 2:

2-[1-(2-Ethoxycarbonylethyl)-imidazolidinylidene]propanedinitrile (Compound A):

Compound Aa (28 g) obtained in step 1 of Reference Example 1 was dissolved in pyridine (170 mL), and methanesulfonyl chloride (17 mL) was added thereto under stirring while ice-cooling. The stirring was continued for 20 minutes at that temperature. The solvent was evaporated under reduced pressure. To the residue was added 0.1 mol/L hydrochloric acid, followed by extraction with ethyl acetate. The extract was washed with a saturated aqueous sodium chloride solution and dried over anhydrous magnesium sulfate. The solvent was evaporated under reduced pressure, and the resulting crude sulfonate was dissolved in tetrahydrofuran (280 mL). 1,8-Diazabicyclo[5.4.0]undec-7-ene (DBU) (18 mL) was added thereto under ice-cooling, followed by stirring at room temperature for 1 hour. The solvent was evaporated under reduced pressure, and 1 mol/L hydrochloric acid was added thereto, followed by extraction with ethyl acetate. The extract was washed with a saturated aqueous sodium chloride solution and dried over anhydrous magnesium sulfate. The solvent was evaporated under reduced pressure. The residue was triturated with diethyl ether to give Compound A (22 g, 84%) as a pale yellow oily substance.

¹H NMR (270 MHz, CDCl₃) δ 5.88 (1H, brs), 4.14 (2H, q), 3.97-3.70 (4H, m), 3.58 (2H, t), 2.71 (2H, t), 1.26 (3H, t).

### REFERENCE EXAMPLE 2

### 2-[1-(2-Carboxyethyl)-3-(3,5-dimethylbenzyl)-imidazolidinylidene]propanedinitrile (Compound B)

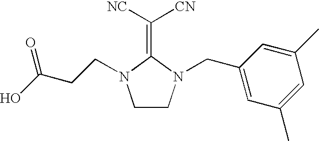

Step 1:

2-[1 -(2-Ethoxycarbonylethyl)-3-(3,5-dimethylbenzyl)-imidazolidinylidene]propanedinitrile (Compound Ba):

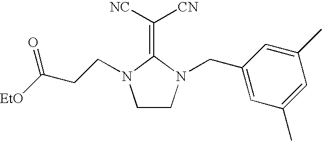

Compound Ba (2.5 g, 95%) was obtained as colorless crystals using Compound A (1.8 g) obtained in Reference Example 1, 3,5-dimethylbenzyl alcohol (1.7 mL), triphenylphosphine (3.0 g), diethyl azodicarboxylate (1.8 mL) and tetrahydrofuran (7.5 mL) as described in Example 14.

¹H NMR (270 MHz, CDCl₃) δ 6.96 (1H, brs), 6.86 (2H, brs), 4.68 (2H, s), 4.17 (2H, q), 3.89 (2H, t), 3.65 (2H, dd), 3.42 (2H, dd), 2.79 (2H, t), 2.31 (6H, s), 1.28 (3H, t).

Step 2:

2-[1-(2-Carboxyethyl)-3-(3,5-dimethylbenzyl)-imidazolidinylidene]propanedinitrile (Compound B):

Compound Ba (2.5 g) obtained in step 1 of Reference Example 2 was dissolved in tetrahydrofuran (7.5 mL), and a 1.5 mol/L aqueous lithium hydroxide solution (7.5 mL) was added thereto, followed by stirring at room temperature for 1 hour. The solvent was evaporated under reduced pressure, and ethyl acetate was added thereto, followed by extraction with a 1 mol/L aqueous potassium hydroxide solution. Ice was added to the aqueous layer, and then 1 mol/L hydrochloric acid was added to adjust the pH to about 1. The mixture was extracted with ethyl acetate, and the extract was washed with an aqueous saturated sodium chloride solution and dried over anhydrous sodium sulfate. The solvent was evaporated under reduced pressure to give Compound B (1.9 g, 82%) as colorless crystals.

¹H NMR (270 MHz, CD₃OD) δ 6.96 (1H, brs), 6.92 (2H, brs), 4.68 (2H, s), 3.85 (2H, t), 3.76-3.64 (2H, m), 3.55-3.43 (2H, m), 2.73 (2H, t), 2.30 (6H, s). The signal which corresponds to a carboxyl group was not observed.

### REFERENCE EXAMPLE 3

### 2-[1-(2-Carboxyethyl)-3-(naphthalenylmethyl)-imidazolidinylidene]propanedinitrile Compound C)

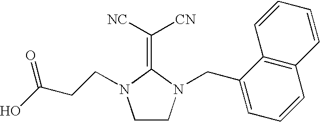

Compound C (1.1 g, 72%) was obtained as colorless crystals using Compound A (1.0 g) obtained in Reference Example 1, 1-naphthalenemethanol (2.0 g), triphenylphosphine (1.7 g), diethyl azodicarboxylate (1.0 mL), tetrahydrofuran (4.5 mL), a 1.5 mol/L aqueous lithium hydroxide solution (4.5 mL), and tetrahydrofuran (4.5 mL) as described in Reference Example 2.

¹H NMR (270 MHz, DMSO-d₆) δ 12.5 (1H, brs), 8.10-7.64 (3H, m), 7.50-7.30 (4H, m), 5.21 (2H, s), 3.97-3.30 (6H, m), 2.69 (2H, t).

### REFERENCE EXAMPLE 4

### 2-[1-(2-Carboxyethyl)-3-(3,5-dichlorobenzyl)-imidazolidinylidene]propanedinitrile (Compound D)

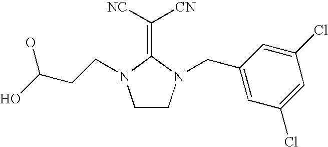

Compound D (1.4 g, 46%) was obtained as colorless crystals using Compound A (2.0 g) obtained in Reference Example 1, 3,5-dichlorobenzyl alcohol (5.3 g), triphenylphosphine (3.4 g), diethyl azodicarboxylate (2.0 mL), tetrahydrofuran (9.0 mL), a 1.5 mol/L aqueous lithium hydroxide solution (9.0 mL), and tetrahydrofuran (9.0 mL) as described in Reference Example 2.

¹H NMR (270 MHz, DMSO-d₆) δ 12.3 (1H, brs), 7.36 (1H, t), 7.31 (2H, d), 4.73 (2H, s), 3.86-3.66 (4H, m), 3.55 (2H, dd), 2.69 (2H, t).

### REFERENCE EXAMPLE 5

### N-Methyl-3-(piperidinomethyl)aniline (Compound E)

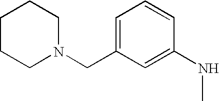

A 28% methanolic sodium methoxide solution (4.0 g) and a methanol solution (4.0 mL) of para-formaldehyde (0.16 g) were added to 1-(3-aminobenzyl)piperidine (0.72 g) obtained by the known process (WO99/32100), followed by stirring at room temperature for 5 hours and 20 minutes. Sodium borohydride (0.15 g) was added thereto, followed by refluxing for 15 minutes. A 1 mol/L aqueous potassium hydroxide solution (5.0 mL) was added thereto, followed by stirring at room temperature for 30 minutes. The reaction mixture was extracted with chloroform, and the extract was dried over anhydrous sodium sulfate. The solvent was evaporated under reduced pressure, and the residue was purified by silica gel preparative thin layer chromatography (chloroform:methanol=5:1) to give Compound E (0.58 g, 75%) as a pale yellow oily substance.

¹H NMR (270 MHz, CDCl₃) δ 7.08 (1H, brt), 6.62 (1H, brd), 6.58 (1H, brs), 6.45 (1H, brd), 3.92 (1H, brs), 3.39 (2H, s), 2.75 (3H, s), 2.37 (4H, m), 1.56 (4H, m), 1.41 (2H, m).

### REFERENCE EXAMPLE 6

### N-Propyl-3-(piperidinomethyl)aniline (Compound F)

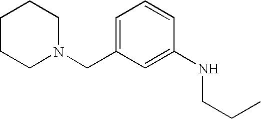

1-(3-Aminobenzyl)piperidine (0.70 g) obtained by the known process (WO99/32100), was dissolved in tetrahydrofuran (15 mL), and propionaldehyde (0.29 mL) and sodium triacetoxyborohydride (1.2 g) were added thereto. After stirring at room temperature for 1.5 hours, propionaldehyde (0.15 mL) was further added thereto, followed by stirring at room temperature for 1.5 hours. Then sodium triacetoxyborohydride (0.55 g) was added thereto, followed by stirring at room temperature for 1 hour. A saturated aqueous sodium bicarbonate solution was added thereto, followed by stirring at room temperature for 30 minutes. The reaction mixture was extracted with chloroform, and the extract was dried over anhydrous sodium sulfate. The solvent was evaporated under reduced pressure. The residue was purified by silica gel preparative thin layer chromatography (chloroform:methanol=5:1) to give Compound F (0.44 g, 52%) as a pale yellow oily substance.

¹H NMR (270 MHz, CDCl₃) δ 7.08 (1H, brt), 6.68-6.50 (2H, m), 6.48 (1H, brd), 4.55 (1H, brs), 3.44 (2H, s), 3.06 (2H, t), 2.41 (4H, m), 1.80-1.30 (8H, m), 0.98 (3H, t).

### REFERENCE EXAMPLE 7

### N-Isopropyl-3-(piperidinomethyl)aniline (Compound G)

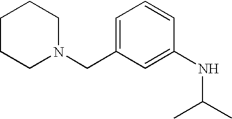

Compound G (0.72 g, 85%) was obtained as a pale yellow oily substance using 1-(3-aminobenzyl)piperidine (0.70 g) obtained by the known process (WO99/32100), acetone (0.45 mL), sodium triacetoxyborohydride (1.7 g), and tetrahydrofuran (15 mL) as described in Reference Example 6.

¹H NMR (270 MHz, CDCl₃) δ 7.06 (1H, brt), 6.64-6.52 (2H, m), 6.44 (1H, brd), 3.63 (1H, brs), 3.60 (1H, septet), 3.38 (2H, s), 2.37 (4H, m), 1.56 (4H, m), 1.41 (2H, m), 1.16 (6H, d).

### REFERENCE EXAMPLE 8

### N-Cyclohexyl-3-(piperidinomethyl)aniline (Compound H)

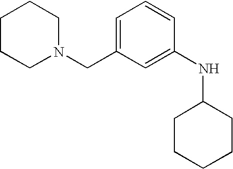

Compound H (0.93 g, 93%) was obtained as colorless crystals using 1-(3-aminobenzyl)piperidine (0.70 g) obtained by the known process (WO99/32100), cyclohexanone (0.52 mL), sodium triacetoxyborohydride (1.7 g) and tetrahydrofuran (15 mL) as described in Reference Example 6.

¹H NMR (270 MHz, CDCl₃) δ 7.04 (1H, brt), 6.64-6.50 (2H, m), 6.43 (1H, brd), 3.72 (1H, brs), 3.38 (2H, s), 3.23 (1H, m), 2.37 (4H, m), 2.01 (2H, m), 1.85-0.95 (14H, m).

### REFERENCE EXAMPLE 9

### N-Benzyl-3-(piperidinomethyl)aniline (Compound I)

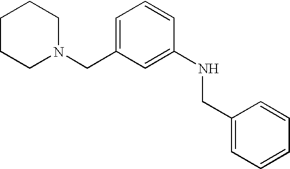

Compound I (0.95 g, 92%) was obtained as a pale yellow oily substance using 1-(3-aminobenzyl)piperidine (0.70 g) obtained by the known process (WO99/32100), benzaldehyde (0.46 mL), sodium triacetoxyborohydride (1.7 g), and tetrahydrofuran (15 mL) as described in Reference Example 6.

¹H NMR (270 MHz, CDCl₃) δ 7.38-7.08 (5H, m), 7.03 (1H, t), 6.68-6.54 (2H, m), 6.43 (1H, brd), 4.20 (2H, s), 4.07 (1H, brs), 3.34 (2H, s), 2.32 (4H, m), 1.52 (4H, m), 1.52 (4H, m), 1.37 (2H, m).

### REFERENCE EXAMPLE 10

### N-[3-(Piperidinomethyl)phenyl]-3-[3-(3,5-dimethylbenzyl)-2-dicyanomethylidene imidazolidin-1-yl]propionamide (Compound J)

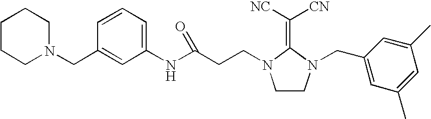

1-(3-Aminobenzyl)piperidine (0.12 g) obtained by the known process (WO99/32100) and Compound B obtained in Reference Example 2 were dissolved in N,N-dimethylformamide (0.70 mL). After ice-cooling, diethylphosphoric cyanide (0.11 mL) and triethylamine (0.20 mL) were added thereto, followed by stirring at room temperature for 2 hours. The solvent was evaporated under reduced pressure, and the residue was purified by silica gel preparative thin layer chromatography (chloroform:methanol=8:1) to give Compound J (0.15 g, 41%) as a pale yellow oily substance.

¹H NMR (270 MHz, CDCl₃) δ 8.94 (1H, brs), 7.51 (1H, brs), 7.45 (1H, brs), 7.18 (1H, brt), 7.04 (1H, brd), 6.91 (1H, brs), 6.85 (2H, brs), 4.65 (2H, s), 4.02-3.86 (2H, m), 3.74-3.58 (2H, m), 3.42 (2H, s), 3.44-3.26 (2H, m), 2.92-2.74 (2H, m), 2.37 (4H, m), 2.27 (6H, s), 1.54 (4H, m), 1.40 (2H, m). MASS (m/e) 497 [(M+H)⁺]

### REFERENCE EXAMPLE 11

### N-(2-Triphenylmethyltetrazol-5-ylmethyl)-N-[3-(piperidinomethyl)phenyl]-3-[3-(3,5-dimethylbenzyl)-2-dicyanomethylidene imidazolidin-1-yl]propionamide (Compound K)

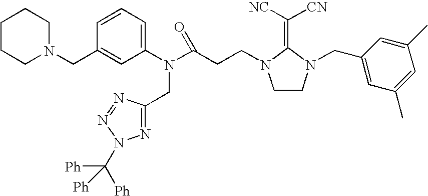

Compound K (0.0083 g, 13%) was obtained as a pale yellow oily substance using Compound J (0.037 g) obtained in Reference Example 10, (2-triphenylmethyltetrazol-5-yl)methyl chloride (0.41 g), potassium hydroxide (0.011 g), and dimethyl sulfoxide (0.30 mL) as described in Example 6.

### REFERENCE EXAMPLE 12

### 2-[1-(2-Carboxyethyl)-3-(methoxymethyl)-imidazolidinylidene]propanedinitrile (Compound L)

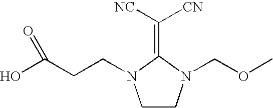

Step 1:

2-[1-(2-Ethoxycarbonylethyl)-3-(methoxymethyl)-imidazolidinylidene]propanedinitrile (Compound La):

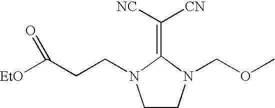

Compound A (1.5 g) obtained in Reference Example 1 was dissolved in tetrahydrofuran (10 mL), and chloromethyl methyl ether (0.55 mL) and a 60% dispersion (0.30 g) of sodium hydride in mineral oil were added thereto, followed by stirring at room temperature for 15 minutes. After ice-cooling, a saturated aqueous ammonium chloride solution was added thereto, followed by extraction with ethyl acetate. The extract was dried over anhydrous sodium sulfate, and the solvent was evaporated under reduced pressure to give crude Compound La (1.9 g) as a pale yellow oily substance.

¹H NMR (270 MHz, CDCl₃) δ 4.92 (2H, s), 4.17 (2H, q), 3.90 (2H, t), 3.84-3.62 (4H, m), 3.38 (3H, s), 2.78 (2H, t), 1.29 (3H, t).

Step 2:

2-[1-(2-Carboxyethyl)-3-methoxymethyl-imidazolidinylidene]propanedinitrile (Compound L)

Crude Compound La (1.9 g) as obtained in step 1 of Reference Example 12 was dissolved in tetrahydrofuran (6.5 mL), and a 1.5 mol/L aqueous lithium hydroxide solution (6.5 mL) was added thereto, followed by stirring at room temperature for 30 minutes. The solvent was evaporated under reduced pressure. Hexane was added thereto, followed by extraction with water. The pH of the aqueous layer was adjusted to about 4 by adding 1 mol/L hydrochloric acid and the mixture was extracted with ethyl acetate. The extract was washed with a saturated aqueous sodium chloride solution and dried over anhydrous sodium sulfate. The solvent was evaporated under reduced pressure to give Compound L (1.3 g, overall yield: 82%) as colorless crystals.

### REFERENCE EXAMPLE 13

### N-Isopropyl-N-[3-(piperidinomethyl)phenyl]-3-(2-dicyanomethylidene imidazolidin-1-yl)propionamide (Compound M)

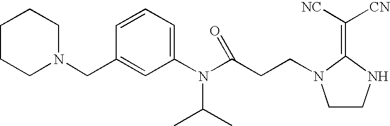

Compound M (0.84 g, 38%) was obtained as a pale yellow oily substance using Compound L (1.3 g) obtained in Reference Example 12, Compound G (1.5 g) obtained in Reference Example 7, thionyl chloride (5.0 mL), a 60% dispersion (0.50 g) of sodium hydride in mineral oil, and tetrahydrofuran (20 mL) as described in Example 3.

¹H NMR (270 MHz, CDCl₃) δ 7.46-7.30 (2H, m), 7.13 (1H, brs), 6.96 (1H, m), 5.40 (1H, brs), 4.96 (1H, septet), 3.98-3.74 (4H, m), 3.68-3.44 (4H, m), 2.41 (4H, m), 2.39-2.23 (2H, m), 1.59 (4H, m), 1.45 (2H, m), 1.06 (6H, d). MASS (m/e) 421 [(M+H)⁺]

### REFERENCE EXAMPLE 14

### 1-(3-Amino-4-methylbenzyl)piperidine (Compound N)

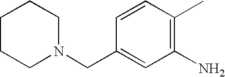

Step 1:

1-(3-Nitro4-methylbenzyl)piperidine (Compound Na):

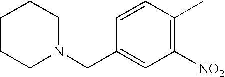

4-Methyl-3-nitrobenzyl chloride (1.0 g) was dissolved in ethanol (22 mL), and piperidine (2.1 mL) was added thereto, followed by stirring at 50° C. for 16 hours. The solvent was evaporated under reduced pressure. A saturated aqueous sodium bicarbonate solution was added thereto, followed by extraction with chloroform. The extract was washed successively with water and a saturated aqueous sodium chloride solution and dried over anhydrous magnesium sulfate. The solvent was evaporated under reduced pressure to give crude Compound Na (0.87 g) as yellow crystals.

Step 2:

1-(3-Amino-4-methylbenzyl)piperidine (Compound N):

Compound Na (0.87 g) obtained in step 1 of Reference Example 14 was dissolved in ethanol (19 mL), and stannic chloride dihydrate (5.0 g) and concentrated hydrochloric acid (4.1 mL) were added thereto, followed by stirring at room temperature for 9 hours. To the reaction mixture was added a 2 mol/L aqueous sodium hydroxide solution, and the solvent was removed by evaporation under reduced pressure. Water was added thereto, followed by extraction with chloroform. The extract was washed successively with water and a saturated aqueous sodium chloride solution and dried over anhydrous magnesium sulfate. The solvent was evaporated under reduced pressure to give Compound N (0.66 g, overall yield: 60%).

¹H NMR (270 MHz, CDCl₃) δ 6.96 (1H, brd), 6.72-6.58 (2H, m), 3.57 (2H, brs), 3.37 (2H, s), 2.37 (4H, m), 2.15 (3H, s), 1.57 (4H, m), 1.43 (2H, m).

### REFERENCE EXAMPLE 15

### N-[2-Methyl-5-(piperidinomethyl)phenyl]-3-[3-(3,5-dichlorobenzyl)-2-dicyanomethylidene imidazolidin-1-yl]propionamide (Compound O)

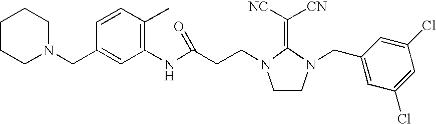

Compound D (0.13 g) obtained in Reference Example 4 and Compound N (0.050 g) obtained in Reference Example 14 were dissolved in dichloromethane (0.24 mL). 1-(3-Dimethylaminopropyl)-3-ethylcarbodiimide hydrochloride (0.069 g) and triethylamine (0.040 mL) were added thereto under ice-cooling, followed by stirring at room temperature for 8.5 hours. Water was added thereto, followed by extraction with ethyl acetate. The extract was dried over anhydrous magnesium sulfate, and the solvent was evaporated under reduced pressure. The residue was purified by silica gel preparative thin layer chromatography (chloroform:methanol=5:1) to give Compound O (0.030 g, 22%) as a pale yellow oily substance.

¹H NMR (270 MHz, CDCl₃) δ 8.27 (1H, brs), 7.72 (1H, brs), 7.32 (1H, t), 7.22-7.10 (2H, m), 7.19 (2H, d), 4.74 (2,H, s), 4.06-3.93 (2H, m), 3.90-3.77 (2H, m), 3.75 (2H, brs), 3.60-3.47 (2H, m), 2.99-2.87 (2H, m), 2.69 (4H, m), 2.28 (3H, s), 1.76 (4H, m), 1.50 (2H, m). MASS (m/e) 551 [(M+H)⁺]

### REFERENCE EXAMPLE 16

### 1-(3-Amino-4-chlorobenzyl)piperidine (Compound P)

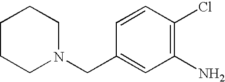

Step 1:

1-(4-Chloro-3-nitrobenzyl)piperidine (Compound Pa):

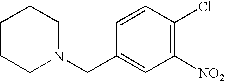

4-Chloro-3-nitrobenzaldehyde (6.0 g) was dissolved in tetrahydrofuran (260 mL), and piperidine (19 mL) and acetic acid (6.4 mL) were added thereto, followed by stirring at room temperature for 40 minutes. After ice-cooling, a mixture of sodium triacetoxyborohydride (21 g), acetic acid (20 mL), and tetrahydrofuran (150 mL) was added thereto, followed by stirring for 1 hour under ice-cooling. A 0.2 mol/L aqueous sodium hydroxide solution was added thereto under ice-cooling, followed by filtration. The residue was washed with ethyl acetate-hexane (1:2) and dried to give Compound Pa (3.3 g, 40%) as orange crystals.

Step 2:

1-(3-Amino-4-chlorobenzyl)piperidine (Compound P):

Compound P (0.49 g, 87%) was obtained as a pale yellow oily substance using Compound Pa(3.3 g) obtained in step 1 of Reference Example 16, stannic chloride dihydrate (3.4 g), concentrated hydrochloric acid (2.8 mL), and ethanol (13 mL) as described in step 2 of Reference Example 14.

¹H NMR (270 MHz, CDCl₃) δ 7.13 (1H, d), 6.77 (1H, d), 6.63 (1H, dd), 4.01 (2H, brs), 3.36 (2H, s), 2.36 (4H, m), 1.57 (4H, m), 1.43 (2H, m).

### REFERENCE EXAMPLE 17

### N-[2-Chloro-5-(piperidinomethyl)phenyl]-3-[3-(3,5-dichlorobenzyl)-2-dicyanomethylidene imidazolidin-1-yl]propionamide (Compound Q)

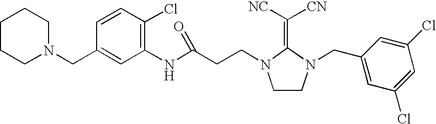

Compound Q (0.22 g, 44%) was obtained as a pale yellow oily substance using Compound D (0.36 g) obtained in Reference Example 4, thionyl chloride (1.0 mL), Compound P (0.19 g) obtained in Reference Example 16, a 60% dispersion (0.068 g) of sodium hydride in mineral oil, and tetrahydrofuran (1.7 mL) as described in Example 3.

¹H NMR (270 MHz, CDCl₃) d8.09 (1H, brs), 7.97 (1H, brs), 7.38-7.23 (2H, m), 7.18 (2H, d), 7.08 (1H, m), 4.74 (2H, brs), 4.07-3.94 (2H, m), 3.88-3.75 (2H, m), 3.52-3.40 (2H, m), 3.43 (2H, brs), 3.01-2.87 (2H, m), 2.36 (4H, m), 1.56 (4H, m), 1.42 (2H, m). MASS (m/e) 571 [(M+H)⁺]

### REFERENCE EXAMPLE 18

### 1-(3-Amino-2-methylbenzyl)piperidine (Compound R)

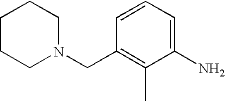

Step 1:

1-(2-Methyl-3-nitrobenzyl)piperidine (Compound Ra):

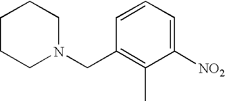

Compound Ra (1.2 g, 94%) was obtained as a yellow oily substance using 2-methyl-3-nitrobenzyl chloride (1.0 g), piperidine (2.1 mL), and ethanol (22 mL) as described in step 1 of Reference Example 14.

Step 2:

1-(3-Amino-2-methylbenzyl)-piperidine (Compound R):

Compound R (0.97 g, 94%) was obtained as a pale yellow oily substance using Compound Ra (1.2 g) obtained in step 1 of Reference Example 18, stannic chloride dihydrate (6.8 g), concentrated hydrochloric acid (5.5 mL), and ethanol (25 mL) as described in Reference Example 14.

¹H NMR (270 MHz, CDCl₃) δ 6.94 (1H, t), 6.71 (1H, d), 6.63 (1H, d), 3.58 (2H, brs), 3.40 (2H, s), 2.36 (4H, m), 2.14 (3H, s), 1.53 (4H, m), 1.42 (2H, m).

### REFERENCE EXAMPLE 19

### N-[2-Methyl-3-(piperidinomethyl)phenyl]-3-[3-(3,5-dichlorobenzyl)-2-dicyanomethylidene imidazolidin-1-yl]propionamide (Compound S)

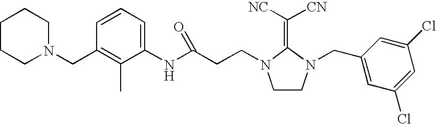

Compound S (0.094 g, 29%) was obtained as a pale yellow oily substance using Compound D (0.33 g) obtained in Reference Example 4, Compound R (0.12 g) obtained in Reference Example 18, 1-(3-dimethylaminopropyl)-3-ethylcarbodiimide hydrochloride (0.17 g), triethylamine (0.091 mL), and dichloromethane (0.60 mL) as described in Reference Example 15.

¹H NMR (270 MHz, CDCl₃) δ 7.62 (1H, brs), 7.51 (1H, m), 7.34 (1H, brt), 7.18 (2H, brd), 7.18-7.05 (2H, m), 4.74 (2H, s), 4.06-3.92 (2H, m), 3.88-3.74 (2H, m), 3.53-3.41 (2H, m), 3.39 (2H, s), 2.97-2.84 (2H, m), 2.36 (4H, m), 2.25 (3H, s), 1.53 (4H, m), 1.43 (2H, m). MASS (m/e) 551 [(M+H)⁺]

### REFERENCE EXAMPLE 20

### N-Ethyl-N-[3-[[N′-(tert-butyloxycarbonyl)propylamino]methyl]phenyl]-3-[3-(3,5-dimethylbenzyl)-2-dicyanomethylidene imidazolidin-1-yl]propionamide (Compound T)

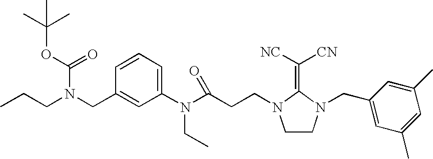

Step 1:

3-(Propylaminomethyl)nitrobenzene (Compound Ta):

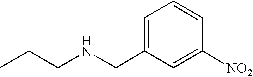

3-Nitrobenzyl chloride (5.3 g) was dissolved in ethanol (53 mL), and n-propylamine (13 mL) was added thereto, followed by stirring at 80° C. for 5 hours. The solvent was evaporated under reduced pressure. A saturated aqueous sodium bicarbonate solution was added thereto, followed by extraction with ethyl acetate. The extract was washed with a saturated aqueous sodium chloride solution and dried over anhydrous magnesium sulfate. The solvent was evaporated under reduced pressure to give crude Compound Ta (6.0 g, 100%) as a pale yellow oily substance.

¹H NMR (270 MHz, CDCl₃) δ 8.22 (1H, s), 8.10 (1H, d), 7.68 (1H, d), 7.49 (2H, dd), 3.90 (2H, s), 2.61 (2H, t), 1.54 (2H, tq), 0.94 (3H, t). MASS (m/e) 195 [(M+H)⁺]

Step 2:

3-[[N-(tert-Butyloxycarbonyl)propylamino]methyl]nitrobenzene (Compound Tb):

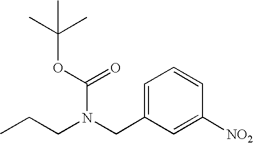

Compound Ta (6.0 g) obtained in step 1 of Reference Example 20 was dissolved in tetrahydrofuran (90 mL), and triethylamine (8.6 mL) and di-tert-butyl dicarbonate (8.8 g) were added thereto, followed by stirring at room temperature for 2 hours. The solvent was evaporated under reduced pressure, and the residue was purified by silica gel column chromatography (hexane:ethyl acetate=10:1) to give Compound Tb (8.6 g, 95%).

¹H NMR (270 MHz, CDCl₃) δ 8.18-8.01 (2H, m), 7.67-7.43 (2H, m), 4.51 (2H, brs), 3.18 (2H, brs), 1.65-1.25 (11H, m), 0.87 (3H, t).

Step 3:

3-[[N-(tert-Butyloxycarbonyl)propylamino]methyl]aniline (Compound Tc):

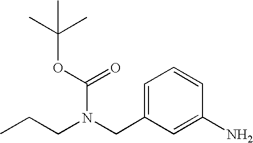

Compound Tb (1.0 g) obtained in step 2 of Reference Example 20 was dissolved in ethanol (10 mL), and 10% palladium carbon (water content: 50%) (0.20 g) was added thereto, followed by stirring under hydrogen atmosphere for 10 hours. The catalyst was removed, and the solvent was evaporated under reduced pressure. The residue was purified by flash silica gel column chromatography (hexane:ethyl acetate=5:1 to 4:1) to give Compound Tc (0.77 g, 86%) as a pale yellow oily substance.

¹H NMR (270 MHz, CDCl₃) δ 7.09 (1H, dd), 6.67-6.43 (3H, m), 4.34 (2H, brs), 3.64 (2H, brs), 3.10 (2H, brs), 1.65-1.25 (1H, m), 0.84 (3H, t).

Step 4:

N-Ethyl-3-[[N-(tert-butyloxycarbonyl)propylamino]methyl]aniline (Compound Td):

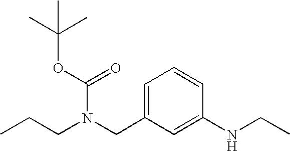

Compound Td (0.50 g, 78%) was obtained as a pale yellow oily substance using Compound Tc (0.58 g) obtained in step 3 of Reference Example 20, acetaldehyde (0.11 mL), sodium triacetoxyborohydride (0.70 g), and tetrahydrofuran (8.7 mL) as described in Reference Example 6.

¹H NMR (270 MHz, CDCl₃) δ 7.11 (1H, dd), 6.60-6.43 (3H, m), 4.35 (2H, brs), 3.54 (1H, brs), 3.14 (2H, q), 1.65-1.27 (11H, m), 1.25 (3H, t), 0.84 (3H, t). MASS (m/e) 293 [(M+H)⁺]

Step 5:

N-Ethyl-N-[3-[[N′-(tert-butyloxycarbonyl)propylamino]methyl]phenyl]-3-[3-(3,5-dimethylbenzyl)-2-dicyanomethylidene imidazolidin-1-yl]propionamide (Compound T):

Compound T (0.18 g, 55%) was obtained as a pale yellow oily substance using Compound B (0.27 g) obtained in Reference Example 2, Compound Td (0.16 g) obtained in step 4 in Reference Example 20, thionyl chloride (1.3 mL), triethylamine (0.15 mL), and tetrahydrofuran (3.2 mL) as described in Example 1.

¹H NMR (270 MHz, CDCl₃) δ 7.43 (1H, m), 7.37-7.18 (1H, m), 7.13-7.01 (2H, m), 6.95 (1H, brs), 6.86 (2H, brs), 4.65 (2H, brs), 4.47 (2H, brs), 3.90-3.60 (6H, m), 3.48-3.32 (2H, m), 3.19 (2H, brs), 2.54-2.38 (2H, m), 2.31 (6H, s), 1.67-1.33 (11H, m), 1.11 (3H, t), 0.87 (3H, t). MASS (m/e) 599 [(M+H)+]

### REFERENCE EXAMPLE 21

### tert-Butyldimethylsilyl 3-(N-isopropylamino)benzyl ether (Compound V)

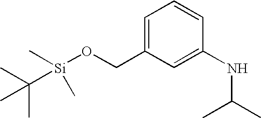

Step 1:

3-(N-Isopropylamino)benzyl alcohol (Compound Va):

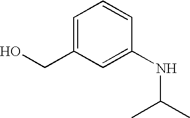

3-Aminobenzyl alcohol (7.0 g) and acetone (8.3 mL) were dissolved in tetrahydrofuran (500 mL), followed by stirring at room temperature for 1 hour. Sodium triacetoxyborohydride (24 g) was added thereto, followed by stirring at room temperature for 4 hours. An aqueous sodium bicarbonate solution was added thereto, followed by extraction with chloroform. The extract was dried over potassium carbonate, and the solvent was evaporated under reduced pressure. The residue was purified by silica gel column chromatography (hexane:ethyl acetate=4:1) to give Compound Va (9.6 g, 100%) as a colorless oily substance.

Step 2:

tert-Butyldimethylsilyl 3-(N-isopropylamino)benzyl ether (Compound V):

Compound Va (9.6 g) obtained in step 1 of Reference Example 21 and triethylamine (16 mL) were dissolved in dichloromethane (100 mL). After ice-cooling, tert-butyldimethylsilyl chloride (18 g) was added thereto, followed by stirring at room temperature for 12 hours. An aqueous sodium bicarbonate solution was added thereto, followed by extraction with ethyl acetate. The extract was dried over anhydrous sodium sulfate, and the solvent was evaporated under reduced pressure. The residue was purified by silica gel column chromatography (hexane:ethyl acetate=4:1) to give Compound V (14 g, 87%) as a pale yellow oily substance.

¹H NMR (270 MHz, CDCl₃) δ 7.12 (1H, dd), 6.64-6.61 (2H, m), 6.48 (1H, brd), 4.69 (2H, s), 3.65 (1H, septet), 3.42 (1H, brs), 1.23 (6H, d), 0.97 (9H, s), 0.12 (6H, s).

### REFERENCE EXAMPLE 22

### N-Isopropyl-N-[3-(chloromethyl)phenyl]-3-[3-(3,5-dimethylbenzyl)-2-dicyanomethylidene imidazolidin-1-yl]propionamide (Compound W)

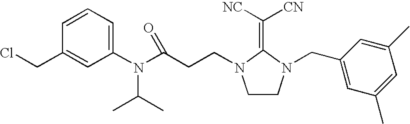

Step 1:

N-Isopropyl-N-[3-(ten-butyldimethylsilyloxymethyl)phenyl]-3-[3-(3,5-dimethylbenzyl)-2-dicyanomethylidene imidazolidin-1-yl]propionamide (Compound Wa):

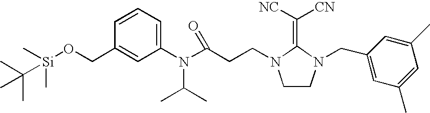

Crude Compound Wa was obtained as a pale yellow oily substance using Compound B (1.0 g) obtained in Reference Example 2, thionyl chloride (10 mL), Compound V (1.3 g) obtained in step 2 of Reference Example 21, a 60% dispersion (0.19 g) of sodium hydride in mineral oil, and tetrahydrofuran (50 mL) as described in Example 3.

Step 2:

N-Isopropyl-N-[3-(hydroxymethyl)phenyl]-3-[3-(3,5-dimethylbenzyl)-2-dicyanomethylidene imidazolidin-1-yl]propionamide (Compound Wb):

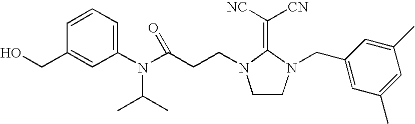

Compound Wa obtained in step 1 of Reference Example 22 was dissolved in tetrahydrofuran (10 mL). A 1 mol/L solution (4.0 mL) of tetrabutylammonium fluoride in tetrahydrofuran was added thereto under ice-cooling, followed by stirring for 1 hour. An aqueous sodium bicarbonate solution was added thereto, followed by extraction with chloroform. The extract was dried over anhydrous sodium sulfate, and the solvent was evaporated under reduced pressure. The residue was purified by silica gel column chromatography (chloroform:methanol=100:1 to 100:3) to give Compound Wb (0.84 g, overall yield: 57%).

Step 3:

N-Isopropyl-N-[3-(chloromethyl)phenyl]-3-[3-(3,5-dimethylbenzyl)-2-dicyanomethylidene imidazolidin-1-yl]propionamide (Compound W):

Compound Wb (0.48 g) obtained in step 2 of Reference Example 22, triethylamine (0.28 mL), and 4-dimethylaminopyridine (0.040 g) were dissolved in dichloromethane (50 mL). p-Toluenesulfonic chloride (0.25 g) was added thereto under ice-cooling, followed by stirring at room temperature for 12 hours. After ice-cooling, an aqueous sodium bicarbonate solution was added thereto, followed by extraction with chloroform. The extract was dried over anhydrous sodium sulfate, and the solvent was evaporated under reduced pressure. The residue was purified by silica gel column chromatography (ethyl acetate) to give Compound W (0.34 g, 78%) as a pale yellow oily substance.

¹H NMR (270 MHz, CDCl₃) δ 7.49-7.47 (2H, m), 7.19 (1H, s), 7.12-7.08 (1H, m), 6.96 (1H, s), 6.86 (2H, s), 4.97 (1H, septet), 4.66 (4H, brs), 3.81 (2H, t), 3.69-3.63 (2H, m), 3.42-3.36 (2H, m), 2.36 (2H, t), 2.31 (6H, s), 1.08 (6H, d).

### REFERENCE EXAMPLE 23

### N-(3-Methoxycarbonylbenzyl)-3-(piperidinomethyl)aniline (Compound X)

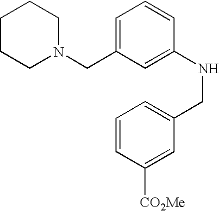

1-(3-Aminobenzyl)piperidine (0.52 g) obtained by the known process (WO99/32100) was dissolved in tetrahydrofuran (4.0 mL) and N,N-dimethylformamide (1.0 mL), and methyl 3-(bromomethyl)benzoate (0.63 g) and a 60% dispersion (0.013 g) of sodium hydride in mineral oil were added thereto, followed by stirring at 50° C. for 3.5 hours. The mixture was allowed to stand for cooling to room temperature, and then a saturated aqueous sodium bicarbonate solution and water were added thereto, followed by extraction with chloroform. The extract was dried over anhydrous sodium sulfate, and the solvent was evaporated under reduced pressure. The residue was purified by silica gel preparative thin layer chromatography (chloroform:methanol=5:1) to give Compound X (0.67 g, 72%) as a pale yellow oily substance.

¹H NMR (270 MHz, CDCl₃) δ 8.03 (1H, m), 7.91 (1H, m), 7.56 (1H, m), 7.38 (1H, t), 7.08 (1H, t), 6.71 (1H, m), 6.65 (1H, brd), 6.51 (1H, brdd), 4.48 (1H, brs), 4.37 (2H, brs), 3.89. (3H, s), 3.49 (2H, brs), 2.45 (4H, m), 1.61 (4H, m), 1.43 (2H, m).

### REFERENCE EXAMPLE 24

### N-(3-Methoxycarbonyl-α-methylbenzyl)-3-(piperidinomethyl)aniline (Compound Y)

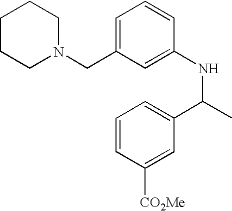

1-(3-Aminobenzyl)piperidine (1.1 g) obtained by the known process (WO99/32100) and methyl 3-acetylbenzoate (1.0 g) obtained by the known method (J. Med. Chem., 13: 674-680 (1970)) were dissolved in dichloromethane (4.0 mL) and acetic acid (1.2 mL), and borane-pyridine complex (a 8 mol/L solution in pyridine; 0.71 mL) was added thereto, followed by stirring at room temperature for 19 hours. Water and a saturated aqueous sodium bicarbonate solution were added thereto to adjust the pH to about 9, followed by stirring at room temperature for 30 minutes and extraction with chloroform. The extract was dried over anhydrous sodium sulfate, and the solvent was evaporated under reduced pressure. The residue was purified by silica gel preparative thin layer chromatography (chloroform:methanol=8:1) to give Compound Y (0.53 g, 27%) as a pale yellow oily substance.

¹H NMR (270 MHz, CDCl₃) δ 8.05 (1H, m), 7.88 (1H, m), 7.56 (1H, m), 7.35 (1H, t), 7.00 (1H, t), 6.64-6.48 (2H, m), 6.37 (1H, m), 4.53 (1H, brq), 4.24 (1H, brs), 3.88 (3H, s), 3.40 (2H, brs), 2.34 (4H, m), 1.56 (4H, m), 1.50 (3H, d), 1.39 (2H, m).

### REFERENCE EXAMPLE 25

### N-(5-Methoxycarbonylfurfuryl)-3-(piperidinomethyl)aniline (Compound Z)

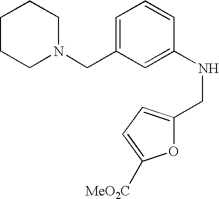

Compound Z (0.28 g, 81%) was obtained as a pale yellow oily substance using 1-(3-aminobenzyl)piperidine (0.20 g) obtained by the known process (WO99/32100), methyl 5-formyl-2-furoate (0.24 g) obtained by the known method (J. Med. Chem., 16: 709-710 (1973)), sodium triacetoxyborohydride (1.1 g), acetic acid (0.30 mL) and tetrahydrofuran (5 mL) as described in Reference Example 6.

¹H NMR (270 MHz, CDCl₃) δ 7.13-7.01 (2H, m), 6.72-6.60 (2H, m), 6.51 (1H, m), 6.32 (1H, d), 4.40 (1H, brs), 4.36 (2H, brs), 3.85 (3H, s), 3.40 (2H, brs), 2.38 (4H, m), 1.56 (4H, m), 1.41 (2H, m).

### REFERENCE EXAMPLE 26

### N-(5-Methoxycarbonyl-2-thenyl)-3-(piperidinomethyl)aniline (Compound AA)

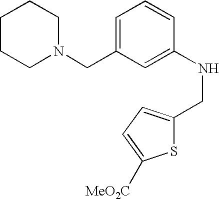

Compound AA (0.19 g, 75%) was obtained as pale yellow crystals using 1-(3-aminobenzyl)piperidine (0.16 g) obtained by the known process (WO99/32100), methyl 5-formyl-2-thiophenecarboxylate (0.12 g) obtained by the known method (J. Heterocycl. Chem., 28: 17-28 (1991)), sodium triacetoxyborohydride (0.89 g), acetic acid (0.25 mL) and tetrahydrofuran (5.0 mL) as described in Reference Example 6.

¹H NMR (270 MHz, CDCl₃) δ 7.63 (1H, d), 7.09 (1H, t), 6.96 (1H, brd), 6.72-6.63 (2H, m), 6.52 (1H, m), 4.49 (2H, brs), 4.37 (1H, brs), 3.82 (3H, s), 3.41 (2H, brs), 2.38 (4H, m), 1.57 (4H, m), 1.42 (2H, m).

### REFERENCE EXAMPLE 27

### N-(4-Hydroxy-3-nitrobenzyl)-3-(piperidinomethyl)aniline (Compound AB)

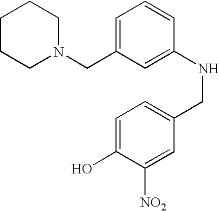

Compound AB (0.25 g, 86%) was obtained as a yellow oily substance using 1-(3-aminobenzyl)piperidine (0.16 g) obtained by the known process (WO99/32100), 4-hydroxy-3-nitrobenzaldehyde (0.14 g), sodium triacetoxyborohydride (0.89 g), acetic acid (0.25 mL) and tetrahydrofuran (5.0 mL) as described in Reference Example 6.

¹H NMR (270 MHz, CDCl₃) d8.67 (1H, brs), 8.01 (1H, d), 7.50 (1H, dd), 7.13-6.97 (2H, m), 6.70 (1H, m), 6.63 (1H, brd), 6.49 (1H, brdd), 4.34 (1H, brs), 4.23 (2H, brs), 3.54 (2H, s), 2.52 (4H, m), 1.63 (4H, m), 1.44 (2H, m).

### REFERENCE EXAMPLE 28

### N-(2-Chlorophenyldiphenylmethyl)-5-(4-bromophenyl)tetrazole (Compound AC)

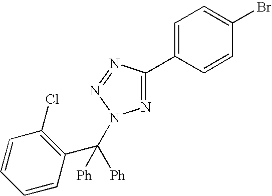

5-(4-Bromophenyl)-1H-tetrazole (0.51 g) was dissolved in tetrahydrofuran (2.5 mL), and triethylamine (0.70 mL) and 2-chlorophenyldiphenylmethyl chloride (0.78 g) were added thereto, followed by stirring at room temperature for 13 hours. Water and a saturated aqueous sodium bicarbonate solution were added thereto to adjust the pH to about 8, followed by extraction with ethyl acetate. The extract was dried over anhydrous sodium sulfate, and the solvent was evaporated under reduced pressure to give Compound AC (1.12 g, 100%) as pale yellow crystals.

¹H NMR (270 MHz, CDCl₃) δ 8.03 (2H, m), 7.57 (2H, m), 7.45 (1H, dd), 7.40-7.12 (12H, m), 6.82 (1H, dd).

### REFERENCE EXAMPLE 29

### N-(2-Chlorophenyldiphenylmethyl)-5-(4-formylphenyl)tetrazole (Compound AD)

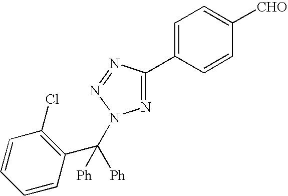

Compound AC (0.56 g) obtained in Reference Example 28 was dissolved in tetrahydrofuran (5.0 mL). After ice-cooling to −78° C., n-butyl lithium (1.59 mol/L in hexane; 0.85 mL) was added thereto, followed by stirring at −78° C. for 1 minute. To the reaction mixture was added N,N-dimethylformamide (0.45 mL), followed by stirring at room temperature for 30 minutes. Water and 1 mol/L hydrochloric acid were added to adjust the pH to about 9, followed by extraction with chloroform. The extract was dried over anhydrous sodium sulfate, and the solvent was evaporated under reduced pressure. The residue was purified by silica gel preparative thin layer chromatography (toluene:ethyl acetate:triethylamine=300:15:1) to give Compound AD (0.39 g, 76%) as a pale yellow oily substance.

¹H NMR (270 MHz, CDCl₃) δ 10.03 (1H, s), 8.34 (2H, m), 7.96 (2H, m), 7.46 (1H, dd), 7.43-7.10 (12H, m), 6.84 (1H, dd).

### REFERENCE EXAMPLE 30

### N-[4-[N-(2-Chlorophenyldiphenylmethyl)tetrazol-5-yl]benzyl]-3-piperidinomethylaniline (Compound AE)

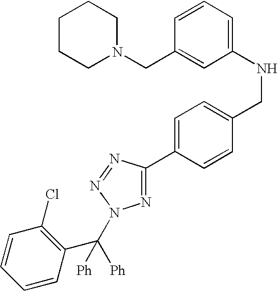

Compound AE (0.42 g, 79%) was obtained as a yellow oily substance using 1-(3-aminobenzyl)piperidine (0.20 g) obtained by the known process (WO99/32100), Compound AD (0.39 g) obtained in Reference Example 29, sodium triacetoxyborohydride (1.1 g), acetic acid (0.30 mL) and tetrahydrofuran (6.0 mL) as described in Reference Example 6.

¹H NMR (270 MHz, CDCl₃) δ 8.11 (2H, m), 7.52-7.10 (15H, m), 7.06 (1H, t), 6.82 (1H, dd), 6.70-6.54 (2H, m), 6.48 (1H, brdd), 4.34 (2H, brs), 4.20 (1H, brs), 3.41 (2H, brs), 2.36 (4H, m), 1.54 (4H, m), 1.36 (2H, m).

### REFERENCE EXAMPLE 31

### N-[4-[N-(2-Chlorophenyldiphenylmethyl)tetrazol-5-yl]benzyl]-N-[3-(piperidinomethyl)phenyl]-3-[3-(3,5-dimethylbenzyl)-2-dicyanomethylidene imidazolidin-1-yl]propionamide (Compound AF)

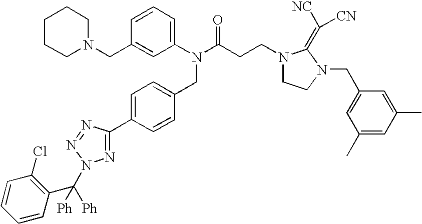

Compound AF (0.50 g, 79%) was obtained as a pale yellow oily substance using Compound B (0.27 g) obtained in Reference Example 2, Compound AE (0.42 g) obtained in Reference Example 30, thionyl chloride (1.0 mL), a 60% dispersion (0.050 g) of sodium hydride in mineral oil and tetrahydrofuran (5.0 mL) as described in Example 3.

¹H NMR (270 MHz, CDCl₃) δ 8.06 (2H, m), 7.43 (1H, dd), 7.40-7.12 (16H, m), 7.01 (1H, brs), 6.97 (1H, m), 6.92 (1H, brs), 6.86 (2H, brs), 6.81 (1H, dd), 4.93 (2H, brs), 4.63 (2H, s), 3.93-3.78 (2H, m), 3.72-3.55 (2H, m), 3.44 (2H, brs), 3.43-3.28 (2H, m), 2.63-2.46 (2H, m), 2.27 (6H, s), 2.27 (4H, m), 1.47 (4H, m), 1.31 (2H, m).

### REFERENCE EXAMPLE 32

### N-(2-Chlorophenyldiphenylmethyl)-5-(3-bromophenyl)tetrazole (Compound AG)

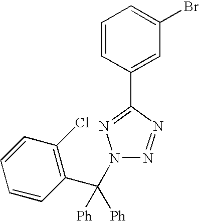

Compound AG (2.3 g, 100%) was obtained as pale yellow crystals using 5-(3-bromophenyl)-1H-tetrazole (1.0 g), 2-chlorophenyldiphenylmethyl chloride (1.6 g), triethylamine (1.4 mL) and tetrahydrofuran (5.0 mL) as described in Reference Example 28.

¹H NMR (270 MHz, CDCl₃) δ 8.31 (1H, m), 8.10 (1H, m), 7.52 (1H, m), 7.44 (1H, dd), 7.40-7.12 (13H, m), 6.83 (1H, dd).

### REFERENCE EXAMPLE 33

### N-(2-Chlorophenyldiphenylmethyl)-5-(3-acetylphenyl)tetrazole (Compound AH)

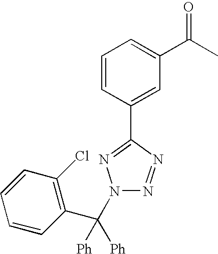

Compound AH (0.43 g, 40%) was obtained as a pale yellow oily substance using Compound AG obtained in Reference Example 32, n-butyl lithium (1.56 mol/L in hexane; 1.8 mL), N,N-dimethylacetamide (1.2 mL) and tetrahydrofuran (10 mL) as described in Reference Example 29.

¹H NMR (270 MHz, CDCl₃) δ 8.75 (1H, m), 8.35 (1H, m), 8.01 (1H, m), 7.51 (1H, t), 7.43 (1H, dd), 7.40-7.10 (12H, m), 6.84 (1H, dd), 2.61 (3H, s).

### REFERENCE EXAMPLE 34

### N-[3-[N-(2-Chlorophenyldiphenylmethyl)tetrazol-5-yl]-α-methylbenzyl]-3-piperidinomethylaniline (Compound AI)

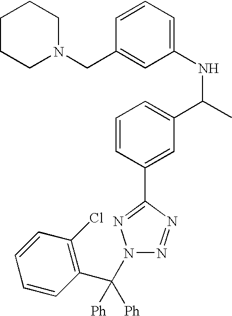

Compound AI (0.086 g, 14%) was obtained as a yellow oily substance using 1-(3-aminobenzyl)piperidine (0.18 g) obtained by the known process (WO99/32100), Compound AH (0.39 g) obtained in Reference Example 33, borane-pyridine complex (a 8 mol/L solution in pyridine; 0.12 mL), dichloromethane (0.70 mL) and acetic acid (0.20 mL) as described in Reference Example 24.

¹H NMR (270 MHz, CDCl₃) δ 8.18 (1H, m), 7.99 (1H, m), 7.54-7.08 (15H, m), 7.01 (1H, t), 6.82 (1H, dd), 7.64-7.52 (2H, m), 7.43 (1H, m), 4.57 (1H, brq), 4.19 (1H, brs), 3.44 (2H, brs), 2.36 (4H, m), 1.55 (4H, m), 1.53 (3H, d), 1.34 (2H, m).

### REFERENCE EXAMPLE 35

### N-[3-[N-(2-Chlorophenyldiphenylmethyl)tetrazol-5-yl]-α-methylbenzyl]-N-[3-(piperidinomethyl)phenyl]-3-[3-(3,5-dimethylbenzyl)-2-dicyanomethylidene imidazolidin-1-yl]propionamide (Compound AJ)

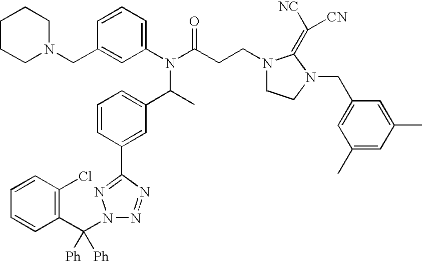

Compound AJ (0.058 g, 46%) was obtained as a pale yellow oily substance using Compound B (0.055 g) obtained in Reference Example 2, Compound AI (0.085 g) obtained in Reference Example 34, thionyl chloride (0.5 mL), a 60% dispersion (0.012 g) of sodium hydride in mineral oil and tetrahydrofuran (1.0 mL) as described in Example 3.

¹H NMR (270 MHz, CDCl₃) δ 8.20-6.10 (26H, m), 4.90-4.30 (2H, m), 4.10-3.05 (8H, m), 3.00-1.90 (12H, m), 1.90-1.00 (9H, m).

### REFERENCE EXAMPLE 36

### N-[[N′-[[2-(Trimethylsilyl)ethoxy]methyl]benzotriazol-5-yl]methyl]-3-(piperidinomethyl)aniline (Compound AK)

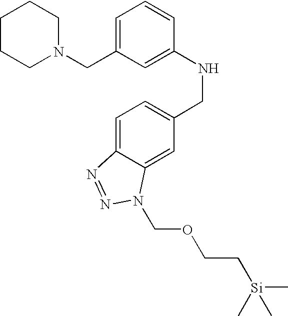

Step: 1

Ethyl 3-[[2-(Trimethylsilyl)ethoxy]methyl]-3H-benzotriazole-5-carboxylate (Compound AKa):

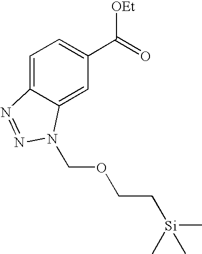

Ethyl 1H-benzotriazole-5-carboxylate (3.0 g) obtained by the known method (Synth. Commun., 23: 2019-2025 (1993)) was dissolved in tetrahydrofuran (30 mL), and [2-(trimethylsilyl)ethoxy]methyl chloride (2.9 mL) and a 60% dispersion (0.79 g) of sodium hydride in mineral oil were added thereto, followed by stirring at room temperature for 20 minutes. Water was added thereto, followed by extraction with ethyl acetate. The extract was washed with water and dried over anhydrous sodium sulfate. The solvent was evaporated under reduced pressure to give crude Compound AKa (4.8 g) as a pale yellow oily substance.

Step 2:

5-Hydroxymethyl-N-[[2-(trimethylsilyl)ethoxy]methyl]benzotriazole (Compound AKb):

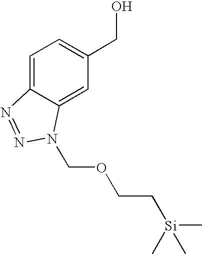

Crude Compound AKa (4.8 g) obtained in step 1 of Reference Example 36 was dissolved in tetrahydrofuran (7.5 mL), and lithium aluminum hydride (1.7 g) was added thereto, followed by refluxing for 12 hours. The mixture was allowed to stand for cooling to room temperature, and then water (1.8 mL) and a 15% aqueous sodium hydroxide solution (1.8 mL) and water (6.0 mL) were added thereto in this order, followed by stirring at room temperature for 20 minutes. Then, the mixture was dried over anhydrous sodium sulfate, and the solvent was evaporated under reduced pressure. The residue was purified by silica gel preparative thin layer chromatography (toluene:ethyl acetate=1:3) to give Compound AKb (0.36 g, 8% by two steps) as a pale yellow oily substance.

Step 3:

5-Formyl-N-[[2-(trimethylsilyl)ethoxy]methyl]benzotriazole (Compound AKc):

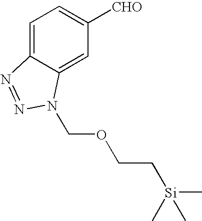

Compound AKb (0.36 g) obtained in step 2 of Reference Example 36 was dissolved in chloroform (80 mL), and manganese dioxide (2.8 g) was added thereto, followed by stirring at room temperature for 4 hours. The reaction mixture was filtered, and the solvent was evaporated from the filtrate under reduced pressure to give crude Compound AKc (0.40 g) as a pale yellow oily substance.

Step 4:

N-[[N′ [[2-(Trimethylsilyl)ethoxy]methyl]benzotriazol-5-yl]methyl]-3-(piperidinomethyl)aniline (Compound AK):

Compound AK (0.15 g, 25% by two steps) was obtained as a pale yellow oily substance using 1-(3-aminobenzyl)piperidine (0.25 g) obtained by the known process (WO99/32100), crude Compound AKc (0.40 g) obtained in step 3 of Reference Example 36, sodium triacetoxyborohydride (1.4 g), acetic acid (0.40 mL) and tetrahydrofuran (20 mL) as described in Reference Example 6.

¹H NMR (270 MHz, CDCl₃) δ 8.02 (1H, d), 7.69 (1H, brs), 7.41 (1H, dd), 7.09 (1H, t), 6.73 (1H, brs), 6.66 (1H, brd), 6.52 (1H, brdd), 5.94 (2H, s), 4.53 (2H, brs), 4.25 (1H, brs), 3.56 (2H, m), 3.47 (2H, brs), 2.43 (4H, m), 1.58 (4H, m), 1.42 (2H, m), 0.88 (2H, m), −0.07 (9H, s).

### REFERENCE EXAMPLE 37

### N-[[N-[[2-(Trimethylsilyl)ethoxy]methoxy]benzotriazol-5-yl]methyl]-N-[3-(piperidinomethyl)phenyl]-3-[3-(3,5-dimethylbenzyl)-2-dicyanomethylidene imidazolidin-1-yl]propionamide (Compound AL)

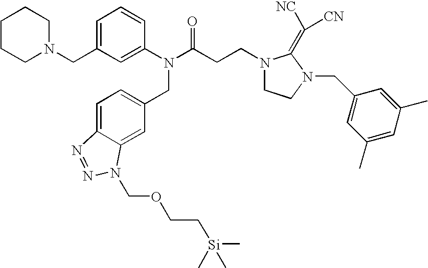

Compound AL (0.23 g, 94%) was obtained as a pale yellow oily substance using Compound B (0.13 g) obtained in Reference Example 2, Compound AK (0.15 g) obtained in Reference Example 36, thionyl chloride (0.5 mL), a 60% dispersion (0.028 g) of sodium hydride in mineral oil and tetrahydrofuran (2.0 mL) as described in Example 3.

¹H NMR (270 MHz, CDCl₃) δ 7.95 (1H, d), 7.49 (1H, brs), 7.33-7.22 (3H, m), 7.05 (1H, brs), 6.95 (1H, brs), 6.92 (1H, m), 6.86 (2H, brs), 5.91 (2H, s), 5.06 (2H, brs), 4.64 (2H, s), 3.93-3.80 (2H, m), 3.76-3.30 (8H, m), 2.63-2.50 (2H, m), 2.30 (6H, s), 2.27 (4H, m), 1.48 (4H, m), 1.42 (2H, m), 0.86 (2H, m), −0.07 (9H, s). MASS (m/e) 758 [(M+H)⁺]

### REFERENCE EXAMPLE 38

### 2,3,5-Trimethylphenyl trifluoromethanesulfonate (Compound AM)

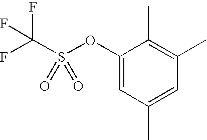

2,3,5-Trimethylphenol (5.0 g) was dissolved in pyridine (64 mL), and after ice-cooling, trifluoromethanesulfonic acid anhydride (9.3 mL) was added thereto, followed by stirring at room temperature for 1 hour. A saturated aqueous ammonium chloride solution was added thereto, followed by extraction with ethyl acetate. The extract was washed with water and a saturated aqueous ammonium chloride solution in this order and dried over anhydrous sodium sulfate, and the solvent was evaporated under reduced pressure. The residue was purified by silica gel column chromatography (hexane:ethyl acetate=50:1) to give Compound AM (9.8 g, 100%) as a pale yellow oily substance.

¹H NMR (270 MHz, CDCl₃) δ 6.97 (1H, brs), 6.89 (1H, brs), 2.30 (3H, brs), 2.27 (3H, brs), 2.21 (3H, brs).

### REFERENCE EXAMPLE 39

### Methyl 2,3,5-trimethylbenzoate (Compound AN)

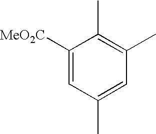

Compound AM (5.0 g) obtained in Reference Example 38 was dissolved in methanol (37 mL) and dimethyl sulfoxide (55 mL), followed by ultrasonic treatment for 20 minutes. Then, triethylamine (6.7 mL), palladium (II) acetate (0.54 g) and 1,3-bis(diphenylphosphino)propane (dppp; 1.2 g) were added thereto, followed by stirring at 60° C. for 4 hours in a carbon monoxide atmosphere. The mixture was allowed to stand for cooling to room temperature, and then the solvent was evaporated under reduced pressure. The residue was purified by florisil column chromatography (ethyl acetate) and silica gel column chromatography (hexane:ethyl acetate=10:1) to give Compound AN (2.8 g, 85%) as a pale yellow oily substance.

¹H NMR (270 MHz, CDCl₃) δ 7.43 (1H, brs), 7.10 (1H, brs), 3.88 (3H, s), 2.40 (3H, brs), 2.29 (3H, brs), 2.28 (3H, brs).

### REFERENCE EXAMPLE 40

### 2,3,5-Trimethylbenzyl Alcohol (Compound AO)

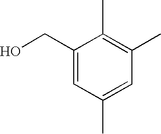

Compound AN (0.50 g) obtained in Reference Example 39 was dissolved in tetrahydrofuran (5.0 mL), and then lithium aluminum hydride (0.16 g) was added thereto, followed by stirring at room temperature for 1 hour. Water (0.20 mL), a 15% aqueous sodium hydroxide solution (0.20 mL) and water (0.60 mL) were added thereto in this order, followed by stirring at room temperature for 20 minutes. After extraction with ethyl acetate, the extract was dried with anhydrous sodium sulfate, and the solvent was evaporated under reduced pressure to give Compound AO (0.32 g, 76%) as a pale yellow oily substance.

¹H NMR (270 MHz, CDCl₃) δ 7.01 (1H, brs), 6.95 (1H, brs), 4.67 (2H, brs), 2.29 (3H, brs), 2.26 (3H, brs), 2.22 (3H, brs), 1.85 (1H, brs).

### REFERENCE EXAMPLE 41

### 3,5-Bis(ethoxycarbonyl)-1-isopropylpyrazole (Compound AP)

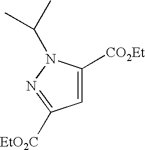

Diethyl 3,5-pyrazoledicarboxylate (3.0 g) obtained in the known method (J. Org. Chem., 64: 6135-6146 (1999)) was dissolved in N,N-dimethylformamide (15 mL), and a 60% dispersion (0.62 g) of sodium hydride in mineral oil and isopropyl iodide (2.1 mL) were added thereto, followed by stirring at room temperature for 3 hours. Water was added thereto, followed by extraction with ethyl acetate. The extract was dried over anhydrous sodium sulfate, and the solvent was evaporated under reduced pressure to give Compound AP (3.6 g, 99%) as pale yellow crystals.

¹H NMR (270 MHz, CDCl₃) δ 7.32 (1H, s), 5.57 (1H, septet), 4.40 (2H, q), 4.35 (2H, q), 1.54 (6H, d), 1.393 (3H, t), 1.388 (3H, t).

### REFERENCE EXAMPLE 42

### Methyl 5-hydroxymethyl-1-isopropylpyrazole-3-carboxylate (Compound AQ)

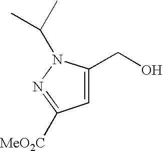

Step 1:

1-Isopropyl-3-methoxycarbonylpyrazole-5-carboxylic acid (Compound AQa):

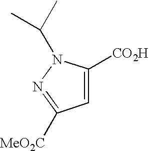

Compound AP (3.6 g) obtained in Reference Example 41 was dissolved in methanol (26 mL), and potassium hydroxide (a 2.0 mol/L solution in methanol; 6.4 mL) was added thereto, followed by stirring at room temperature for 2 days. The solvent was evaporated under reduced pressure, and water was added thereto, followed by washing with ethyl acetate. The aqueous layer was acidified by adding 1 mol/L hydrochloric acid, followed by extraction with ethyl acetate. The extract was dried over anhydrous sodium sulfate, and the solvent was evaporated under reduced pressure to give crude Compound AQa (2.2 g) as pale yellow crystals.

Step 2:

Methyl 5-hydroxymethyl-1-isopropylpyrazole-3-carboxylate (Compound AQ):

Crude Compound AQa (2.2 g) obtained in step 1 of Reference Example 42 was dissolved in tetrahydrofuran (10 mL), borane-tetrahydrofuran complex (a 1.0 mol/L solution in tetrahydrofuran; 1.0 mL) was added thereto, followed by stirring at room temperature for 1 day, and then borane-dimethyl sulfide complex (a 10 mol/L solution in dimethyl sulfide; 1.0 mL) was added thereto, followed by stirring at room temperature. Water was added thereto, followed by extraction with ethyl acetate, the extract was dried over anhydrous sodium sulfate, and the solvent was evaporated under reduced pressure. The residue was purified by silica gel column chromatography (hexane:ethyl acetate=8:1) to give Compound AQ (0.28 g, overall yield: 10%) as a pale yellow oily substance.

¹H NMR (270 MHz, CDCl₃) δ 6.71 (1H, brs), 4.82-4.58 (3H, m), 3.91 (3H, s), 1.82 (1H, t), 1.55 (6H, d).

### REFERENCE EXAMPLE 43

### Methyl 5-formyl-1-isopropylpyrazole-3-carboxylate (Compound AR)

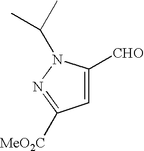

Oxalyl chloride (0.16 mL) was dissolved in dichloromethane (6.0 mL). After cooling to −78° C., dimethyl sulfoxide (a 1.6 mol/L solution in dichloromethane; 2.3 mL) was added thereto, followed by stirring at −78° C. for 15 minutes. To the reaction mixture was added a dichloromethane solution (3 mL) of Compound AQ (0.28 g) obtained in Reference Example 42, followed by stirring at −78° C. for 15 minutes. Triethylamine (0.79 mL) was added thereto, followed by stirring at room temperature for 20 minutes. Water was added thereto, followed by extraction with dichloromethane. The extract was dried over anhydrous sodium sulfate, and the solvent was evaporated under reduced pressure to give Compound AR (0.28 g, 100%) as a pale yellow oily substance.

¹H NMR (270 MHz, CDCl₃) δ 9.86 (1H, s), 7.41 (1H, s), 5.45 (1H, septet), 3.95 (3H, s), 1.55 (6H, d).

### REFERENCE EXAMPLE 44

### N-[(1-Isopropyl-3-methoxycarbonylpyrazol-5-yl)methyl]-3-(piperidinomethyl)aniline (Compound AS)

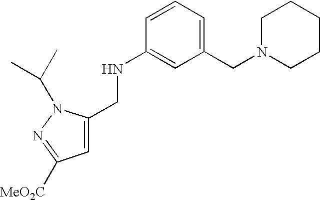

Compound AS (0.37 g, 71%) was obtained as a pale yellow oily substance using 1-(3-aminobenzyl)piperidine (0.28 g) obtained by the known process (WO99/32100), Compound AR (0.28 g) obtained in Reference Example 43, sodium triacetoxyborohydride (0.90 g), acetic acid (0.24.mL) and tetrahydrofuran (5.0 mL) as described in Reference Example 6.

¹H NMR (270 MHz, CDCl₃) δ 7.13 (1H, t), 6.84-6.62 (3H, m), 6.56 (1H, m), 4.62 (1H, septet), 4.34 (2H, brd), 3.96 (1H, brt), 3.88 (3H, s), 3.44 (2H, brs), 2.41 (4H, m), 1.59 (4H, m), 1.54 (6H, d), 1.44 (2H, m).

### REFERENCE EXAMPLE 45

### N-[(4-Methoxycarbonylquinolin-2-yl)methyl]-3-(piperidinomethyl)aniline (Compound AT)

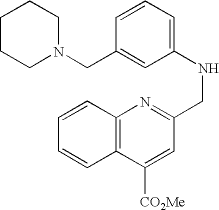

Compound AT (0.36 g, 46%) was obtained as a pale yellow oily substance using 1-(3-aminobenzyl)piperidine (0.38 g) obtained by the known process (WO99/32100), 2-formyl-4-methoxycarbonylquinoline (0.43 g) obtained by the known method (Bull. Soc. Chem. Fr., 789-792 (1976)), sodium triacetoxyborohydride (1.2 g), acetic acid (0.33 mL) and tetrahydrofuran (7.0 mL) as described in Reference Example 6.

¹H NMR (270 MHz, CDCl₃) δ 8.72 (1H, m), 8.16 (1H, m), 7.96 (1H, s), 7.77 (1H, m), 7.63 (1H, m), 7.14 (1H, t), 6.80 (1H, brs), 6.74-6.56 (2H, m), 5.13 (1H, brs), 4.67 (2H, brs), 4.02 (3H, s), 3.46 (2H, brs), 2.40 (4H, m), 1.57 (4H, m), 1.42 (2H, m).

### TEST EXAMPLE

Preparation of CXCR3 Transfectants Cells:

L1/2 cells were grown in RPMI medium 1640, 10% Fetal Clone (Hyclone, Inc., Logan, Utah), 50 U/mL Penicillin/Streptomycin, 1 mmol/L NaPyruvate, and 5.5×10⁻⁵ mol/L β-mercaptoethanol. Media components other than serum were purchased from GibcoBRL (Gaithersburg, Md.). Two days prior to transfection, the L1/2 cells were diluted 1:5 into fresh medium. This resulted in 150 million cells in log phase growth at a concentration of about 1-3 million cells/mL.

CXCR3 DNA and Transfection:

E. coli XL1Blue cells (Stratagene, Inc., La Jolla, Calif.) were transformed with a pCDNA3-based (Invitrogen, San Diego, Calif.) CXCR3 cDNA expression plasmid (Qin, S. et al., J. Clin. Invest., 101: 746-754 (1998), Loetscher, M. et al., J. Exp. Med., 184: 963-969 (1996)) according to the manufacturer's protocol. Transformants were grown at 37° C. while shaking at 250 rpm in 500 mL of LB containing 100 μg/mL Ampicillin. The culture was then collected by centrifugation at 8,000×g, and the plasmid was purified using a Maxi plasmid purification column and protocol (Qiagen, Chatsworth, Calif.). Plasmid concentration and purity were determined using a 1% agarose gel and OD_{260/280} ratios. Plasmid DNA was suspended in ddH₂O, and stored at −20° C. until use.

ScaI endonuclease was used to linearize the CXCR3 expression plasmid. 100 μg of DNA was digested with 10 μl of ScaI for 8 hours at 37° C. following the manufacturer's protocol (GibcoBRL, Cat# 15436-017). 20 μg was used directly in stable transfection (see below). 80 μg was cleaned of proteins and salts with a phenol:chloroform:isoamyl alcohol (25:24:1) extraction, 100% ethanol precipitation (with 0.1 volume NH₄COOH), and a 70% ethanol wash.

Stable transfectants of murine pre-B lymphoma cell line (L1/2) were prepared as described (Ponath, P. D. et al., J. Exp. Med., 183: 2437-2448 (1996)). 25 million L1/2 cells in 0.8 mL of 1×PBS were electroporated with 20 μg of linearized DNA, 20 μg linearized DNA that had been cleaned (see above under Linearization of DNA), or without DNA. Before electroporation, the L1/2 cells and the DNA were incubated for 10 minutes in 50 mL conical tubes (Falcon Model 2070, Becton Dickinson LabWare, Lincoln Park, N.J.) with gentle mixing (swirling) every 2 minutes. The L1/2 cell-DNA mixture was transferred into Gene Pulser cuvettes (BioRad, Richmond, Calif.) with a 0.4 cm electrode gap. The mixture was then electroporated at 250V and 960 μF, with the duration of shock and the actual voltage being measured. After electroporation, the cuvette was left undisturbed for 10 minutes at room temperature. All of the L1/2 cells-DNA mixture was then transferred to a T-25 tissue culture flask (Costar, Cambridge, Mass.), and grown for two days in 10 mL non-selective medium.

Selection:

L1/2 cells expressing CXCR3 were then subjected to selection for neomycin resistance. After two days of growth in non-selective medium, 10 mL of 1.6 g/L G418 (GibcoBRL) was added to the culture for a final concentration of 0.8 g/L (the selective and maintenance concentration). This was then allowed to grow for 10 to 15 days, with fresh selective medium added when cells started to over-grow. Fresh selective medium consisted of RPMI-1640 supplemented with 10% bovine serum and 0.8 g/L G418.

The cell surface expression of CXCR3 was assessed by chemotaxis, and ligand binding and Scatchard analysis was also used to monitor surface expression. After G418 selection, CXCR3 expressing L1/2 cells were selected based on chemotaxis ability. For each electroporation reaction culture, 30 mL (800,000 cells/mL) were collected, and suspended in 600 μl selective medium. Selective medium, 600 μl, containing 10 nmol/L IP-10, was placed into the bottom chamber of BioCoat cell culture plates from Becton Dickinson. 100 μl/well of the L1/2 cells were added into the top chamber of the BioCoat plates. The cells were then left to chemotax overnight in a CO₂ incubator at 37° C. The next day, the top chambers with the non-chemotaxing cells were removed. The cells which chemotaxed were collected from the bottom chamber, transferred into fresh medium, and allowed to grow in a 24-well plate. They were subsequently expanded into a T-25 and then a T-75 flask from Costar.

Transfectants expressing high level of receptors were cloned by limiting dilution. CXCR3 transfected cells were diluted to between 30 cells/mL and 3 cells/mL in selection medium containing G418. Aliquots of the dilutions were added to 96-well tissue culture plates at 100 μl/well. After 14 days at 37° C. and 5% CO₂, wells containing single colonies were identified under an inverted microscope. 50 μL of the cells were then transferred and stained with anti-CXCR3 mAb and analyzed by flow cytometry as described (Qin, S. et al., J. Clin. Invest., 101: 746-754 (1998)). The level of receptor expression correlated with mean fluorescence intensity and cells which expressed high levels of CXCR3 were selected. Once a stable cell line was established, the line was expanded for use, and is referred to herein as CXCR3.L1/2.

CXCR3/IP-10 Radioligand Binding:

CXCR3.L1/2 Membrane Preparation:

CXCR3.L1/2 cells were pelleted by centrifugation and stored at −80° C. The cells were lysed by thawing and resuspending at about 1.5×10⁷ cells/mL in a hypotonic buffer (5 mmol/L HEPES (pH 7.2), 2 mmol/L EDTA, 10 μg/mL each leupeptin, aprotinin, and chymostatin, and 100 μg/mL PMSF (all from Sigma, St. Louis)). Nuclei and cellular debris are removed by centrifugation (500 g to 100 g, at 4° C.) for 10 min. The supernatant was transferred to chilled centrifuge tubes (Nalge, Rochester, N.Y.) and the membrane fraction was recovered by centrifugation (25,000 g at 4° C.) for 45 min. The membrane pellet was resuspended in freezing buffer (10 mmol/L HEPES (pH 7.2), 300 mmol/L Sucrose, 5 μg/mL each of leupeptin, aprotinin, and chymostatin, and 10 μg/mL PMSF). The total protein concentration was determined using a coomassie blue staining protein concentration assay kit (BioRad). The membrane preparations are aliquoted and stored at −80° C. until time of use.

Binding Assay:

CXCR3/IP-10 binding was performed in 96-well polypropylene plates (Costar) in a final volume of 0.1 mL of HBB buffer (50 mmol/L Hepes pH 7.4, 1 mmol/L CaCl₂, 5 mmol/L MgCl₂, 0.02% sodium azide, 0.5% BSA (bovine serum albumin)), containing 1 to 5 μg CXCR3.L1/2 transfectant cell membrane protein and 0.05 to 0.2 nmol/L of ¹²⁵I-labeled IP-10 (NEN, Boston, Mass.). Competition binding experiments were performed by including variable concentrations of unlabeled IP-10 or test compound. Nonspecific binding was determined following the addition of a 250 nmol/L unlabelled IP-10. Samples were incubated for 60 min at room temperature, and bound and free tracer (¹²⁵I-labeled IP-10) were separated by filtration through 96-well GF/B filterplates presoaked in 0.3% polyethyleneimine. The filters were washed in HBB further supplemented with 0.5 mol/L NaCl, dried, and the amount of bound radioactivity determined by liquid scintillation counting. The competition is presented as the percent specific binding as calculated by 100×[(S−B)/(T−B)], where S is the radioactivity bound for each sample, B is background binding, and T is total bound in the absence of competitors. Duplicates were used throughout the experiments.

The results are shown in Tables 5 and 6 below.

TABLE 5

| Compound Number | % inhibition at 10 μmol/L |
|---|---|
| 1 | 92 |
| 2 | 99 |
| 3 | 106 |
| 4 | 109 |
| 5 | 95 |
| 6 | 93 |
| 7 | 83 |
| 8 | 95 |
| 9 | 83 |
| 10 | 55 |
| 11 | 95 |
| 12 | 84 |
| 13 | 67 |

TABLE 6

| Compound Number | % inhibition at 10 μmol/L |
|---|---|
| 14 | 105 |
| 15 | 95 |
| 16 | 95 |
| 17 | 93 |
| 18 | 94 |
| 19 | 101 |
| 20 | 68 |
| 21 | 71 |
| 22 | 93 |
| 23 | 92 |
| 24 | 90 |
| 25 | 107 |
| 26 | 93 |
| 27 | 92 |

While the invention has been described in detail and with reference to specific embodiments thereof, it will be apparent to one skilled in the art that various changes and modifications can be made therein without departing from the spirit and scope thereof.

What is claimed is:

1. An imidazolidine compound represented by the following Formula (I): 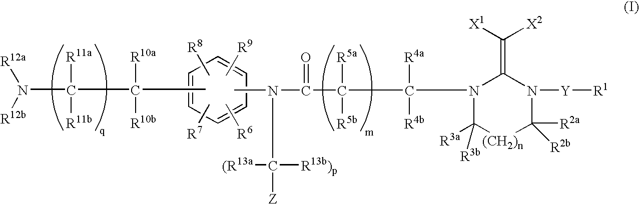
or a physiologically acceptable salt thereof, wherein:
Z is
hydrogen,
halogen,
hydroxy,
—13 COOH,
—CONH₂,
substituted or unsubstituted lower alkyl,
substituted or unsubstituted haloalkyl,
substituted or unsubstituted heteroalkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted polycycloalkenyl,
substituted or unsubstituted lower alkoxy,
substituted or unsubstituted lower alkanoyloxy,
substituted or unsubstituted lower alkanoyl,
substituted or unsubstituted lower alkoxycarbonyl,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group, or
Z and R⁶ taken together form a bond, or
Z and R^{13a} taken together form a bond;
X¹ and X² are each, independently,
hydrogen,
—C,
—NO₂,
—SO₂R^{15a},
—SO₂NR^{15a}R^{15b},
—C(═O)—R^{15a},
—C(═O)—OR^{15a}, or
—C(═O)—NR^{15a}R^{15b}, wherein
R^{15a} and R^{15b} are each, independently,
hydrogen,
substituted or unsubstituted lower alkyl,
substituted or unsubstituted cycloalkyl,
substituted or unsubstituted aryl, or
substituted or unsubstituted aralkyl;
Y is
a bond,
—(C═O)—, or
—(CR^{16a}R^{16b})—, wherein
R^{16a} and R^{16b} are each, independently,
hydrogen,
substituted or unsubstituted lower alkyl,
substituted or unsubstituted cycloalkyl,
substituted or unsubstituted aryl, or
substituted or unsubstituted aralkyl;
R¹ is
substituted or unsubstituted lower alkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted lower alkoxy,
substituted or unsubstituted lower alkanoyloxy,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group;
R^{2a}, R^{2b}, R^{3a}, R^{3b}, R^{4a}, R^{4b}, R^{5a}, and R^{5b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl;
R⁶, R⁷, R⁸, and R⁹ are each, independently,
hydrogen,
hydroxy,
substituted or unsubstituted lower alkyl,
substituted or unsubstituted lower alkoxy,
substituted or unsubstituted lower alkanoyl,
substituted or unsubstituted lower alkanoyloxy,
substituted or unsubstituted lower alkoxycarbonyl,
substituted or unsubstituted aryl,
substituted or unsubstituted heteroaryl,
halogen,
—CN,
—NO₂,
—C(═O)—OR^{17a},
—NR^{17a}R^{17b} or
—C(═O)—NR^{17a}R^{17b}, wherein
R^{17a} and R^{17b} are each, independently,
hydrogen,
substituted or unsubstituted lower alkyl,
substituted or unsubstituted cycloalkyl,
substituted or unsubstituted aryl, or
substituted or unsubstituted aralkyl, or
R^{17a} and R^{17b} taken together with the nitrogen atom to which they are bonded form a substituted or unsubstituted heterocyclic group containing at least one nitrogen atom;
R^{10a}, R^{10b}, R^{11a}, and R^{11b} are each, independently,
hydrogen,
substituted or unsubstituted lower alkyl,
substituted or unsubstituted cycloalkyl,
substituted or unsubstituted aryl,
substituted or unsubstituted aralkyl,
substituted or unsubstituted heteroaralkyl, or
substituted or unsubstituted lower alkoxyalkyl;
R^{12a} and R^{12b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl, or
R^{12a} and R^{12b} taken together with the nitrogen atom to which they are bonded form a substituted or unsubstituted heterocyclic group containing at least one nitrogen atom;
R^{13a} and R^{13b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl,
wherein when p is 2 or more, multiple R^{13a}'s are independently the same or different and multiple R^{13b}'s are independently the same or different;
m is an integer from 0 to 4;
n is an integer from 0 to 6;
p is an integer from 0 to 9; and
q is an integer from 0 to 5

2. The imidazolidine compound according to claim 1 wherein
Z is
hydrogen,
halogen,
hydroxy,
COOH,
CONH₂,
substituted or unsubstituted lower alkyl,
substituted or unsubstituted haloalkyl,
substituted or unsubstituted heteroalkyl,
substituted or unsubstituted cycloalkyl,
substituted or unsubstituted lower alkenyl,
substituted or unsubstituted lower alkoxy,
substituted or unsubstituted lower alkanoyloxy,
substituted or unsubstituted lower alkanoyl,
substituted or unsubstituted lower alkoxycarbonyl,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group, or
Z and R⁶ taken together form a bond, or
Z and R^{13a} taken together form a bond;
X¹ and X² are each, independently,
hydrogen,
—CN,
—NO₂,
C(═O)—R^{15a},
C(═O)—OR^{15a}, or
—C(═O)—NR^{15a}R^{15b}, wherein
R^{15a} and R^{15b} are each, independently,
hydrogen,
substituted or unsubstituted lower alkyl,
substituted or unsubstituted cycloalkyl,
substituted or unsubstituted aryl, or
substituted or unsubstituted aralkyl;
R⁶, R⁷, R⁸, and R⁹ are each, independently,
hydrogen,
substituted or unsubstituted lower alkyl,
substituted or unsubstituted lower alkoxy,
substituted or unsubstituted lower alkanoyl,
substituted or unsubstituted lower alkoxycarbonyl,
substituted or unsubstituted aryl,
substituted or unsubstituted heteroaryl,
halogen,
—CN,
—NO₂,
—C(═O)—OR^{17a},
—NR^{17a}R^{17b}, or
—C(═O)—NR^{17a}R^{17b}, wherein
R^{17a} and R^{17b} are each, independently,
hydrogen,
substituted or unsubstituted lower alkyl,
substituted or unsubstituted cycloalkyl,
substituted or unsubstituted aryl, or
substituted or unsubstituted aralkyl, or
R^{17a} and R^{17b} taken together with the nitrogen atom to which they are bonded form a substituted or unsubstituted heterocyclic group containing at least one nitrogen atom;
m is an integer from 0 to 3;
n is an integer from 0 to 3;
p is an integer from 0 to 8; and
q is an integer from 0 to 3.

3. The imidazolidine compound according to claim 2 wherein
X¹ and X² are each, independently,
hydrogen,
—CN, or
—NO₂;
R¹ is
substituted or unsubstituted lower alkyl,
substituted or unsubstituted cycloalkyl,
substituted or unsubstituted lower alkenyl,
substituted or unsubstituted lower alkoxy,
substituted or unsubstituted lower alkanoyloxy,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group;
R^{2a}, R^{2b}, R^{3a}, R^{3b}, R^{4a}, R^{4b}, R^{5a}, and R^{5b} are each, independently,
hydrogen,
substituted or unsubstituted lower alkyl,
substituted or unsubstituted cycloalkyl,
substituted or unsubstituted aryl, or
substituted or unsubstituted aralkyl;
R⁶, R⁷, R⁸, and R⁹ are each, independently,
hydrogen,
substituted or unsubstituted lower alkyl,
substituted or unsubstituted lower alkoxy,
substituted or unsubstituted heteroaryl,
halogen,
—CN, or
—NO₂:
R^{10a}, R^{10b}, R^{11a}, and R^{11b} are each, independently,
hydrogen,
substituted or unsubstituted lower alkyl,
substituted or unsubstituted cycloalkyl, or
substituted or unsubstituted aryl.

4. A composition comprising the imidazolidine compound according to claim 1 or a pharmaceutically acceptable salt thereof and a pharmaceutically acceptable carrier.

5. A method of inhibiting inflammation in an individual, comprising administering to the individual a therapeutically effective amount of an imidazolidine compound represented by the following Formula (I): 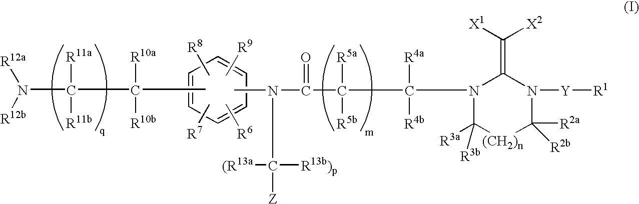
or a physiologically acceptable salt thereof, wherein:
Z is
hydrogen,
halogen,
hydroxy,
—COOH,
—CONH₂,
substituted or unsubstituted lower alkyl,
substituted or unsubstituted haloalkyl,
substituted or unsubstituted heteroalkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted polycycloalkenyl,
substituted or unsubstituted lower alkoxy,
substituted or unsubstituted lower alkanoyloxy,
substituted or unsubstituted lower alkanoyl,
substituted or unsubstituted lower alkoxycarbonyl,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group, or
Z and R⁶ taken together form a bond, or
Z and R^{13a} taken together form a bond;
X¹ and X² are each, independently,
hydrogen,
—CN,
—NO₂,
—SO₂R^{15a},
—SO₂NR^{15a}R^{15b},
—C(═O)—R^{15a},
—C(═O)—OR^{15a}, or
—C(═O)—NR^{15a}R^{15b}, wherein
R^{15a} and R^{15b} are each, independently,
hydrogen,
substituted or unsubstituted lower alkyl,
substituted or unsubstituted cycloalkyl,
substituted or unsubstituted aryl, or
substituted or unsubstituted aralkyl;
Y is
a bond,
—(C═O)—, or
—(CR^{16a}R^{16b})—, wherein
R^{16a} and R^{16b} are each, independently,
hydrogen,
substituted or unsubstituted lower alkyl,
substituted or unsubstituted cycloalkyl,
substituted or unsubstituted aryl, or
substituted or unsubstituted aralkyl;
R¹ is
substituted or unsubstituted lower alkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted lower alkoxy,
substituted or unsubstituted lower alkanoyloxy,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group;
R^{2a}, R^{2b}, R^{3a}, R^{3b}, R^{4a}, R^{4b}, R^{5a}, and R^{5b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl;
R⁶, R⁷, R⁸, and R⁹ are each, independently,
hydrogen,
hydroxy,
substituted or unsubstituted lower alkyl,
substituted or unsubstituted lower alkoxy,
substituted or unsubstituted lower alkanoyl,
substituted or unsubstituted lower alkanoyloxy,
substituted or unsubstituted lower alkoxycarbonyl,
substituted or unsubstituted aryl,
substituted or unsubstituted heteroaryl,
halogen,
—CN,
—NO₂,
—C(═O)—OR^{17a},
—NR^{17a}R^{17b}, or
—C(═O)—NR^{17a}R^{17b}, wherein
R^{17a} and R^{17b} are each, independently,
hydrogen,
substituted or unsubstituted lower alkyl,
substituted or unsubstituted cycloalkyl,
substituted or unsubstituted aryl, or
substituted or unsubstituted aralkyl, or
R^{17a} and R^{17b} taken together with the nitrogen atom to which they are bonded form a substituted or unsubstituted heterocyclic group containing at least one nitrogen atom;
R^{10a}, R^{10b}, R^{11a}, and R^{11b} are each, independently,
hydrogen,
substituted or unsubstituted lower alkyl,
substituted or unsubstituted cycloalkyl,
substituted or unsubstituted aryl,
substituted or unsubstituted aralkyl,
substituted or unsubstituted heteroaralkyl, or
substituted or unsubstituted lower alkoxyalkyl;
R^{12a} and R^{12b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl, or
R^{12a} and R^{12b} taken together with the nitrogen atom to which they are bonded form a substituted or unsubstituted heterocyclic group containing at least one nitrogen atom;
R^{13a} and R^{13b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl,
wherein when p is 2 or more, multiple R^{13a}'s are independently the same or different and multiple R^{13b}'s are independently the same or different;
m is an integer from 0 to 4;
n is an integer from 0 to 6;
p is an integer from 0 to 9; and
q is an integer from 0 to 5

6. The method according to claim 5 wherein said inflammation is a consequence of an autoimmune disease.

7. The method according to claim 5 wherein said inflammation is a consequence of an allergic disease or condition.

8. The method according to claim 5 wherein said inflammation is a consequence of infection.

9. The method according to claim 8 wherein said infection is bacterial, viral, fungal or parasitic.

10. A method of treating an individual having a disease associated with pathogenic leukocyte recruitment and/or activation, comprising administering to the individual a therapeutically effective amount of an imidazolidine compound represented by the following Formula (I): 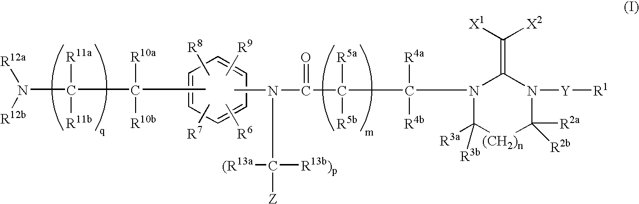
or a physiologically acceptable salt thereof, wherein:
Z is
hydrogen,
halogen,
hydroxy,
—COOH,
—CONH₂,
substituted or unsubstituted lower alkyl,
substituted or unsubstituted haloalkyl,
substituted or unsubstituted heteroalkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted polycycloalkenyl,
substituted or unsubstituted lower alkoxy,
substituted or unsubstituted lower alkanoyloxy,
substituted or unsubstituted lower alkanoyl,
substituted or unsubstituted lower alkoxycarbonyl,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group, or
Z and R⁶ taken together form a bond, or
Z and R^{13a} taken together form a bond;
X¹ and X² are each, independently,
hydrogen,
—CN,
—NO₂,
—SO₂R^{15a},
—SO₂NR^{15a}R^{15b},
—C(═O)—R^{15a},
—C(═O)—OR^{15a}, or
—C(═O)—NR^{15a}R^{15b}, wherein
—R^{15a} and R^{15b} are each, independently,
hydrogen,
substituted or unsubstituted lower alkyl,
substituted or unsubstituted cycloalkyl,
substituted or unsubstituted aryl, or
substituted or unsubstituted aralkyl;
Y is
a bond,
—(C═O)—, or
—(CR^{16a}R^{16b})—, wherein
R^{16a} and R^{16b} are each, independently,
hydrogen,
substituted or unsubstituted lower alkyl,
substituted or unsubstituted cycloalkyl,
substituted or unsubstituted aryl, or
substituted or unsubstituted aralkyl;
R¹ is
substituted or unsubstituted lower alkyl,
substituted or unsubstituted cycloalkyl,
substituted or unsubstituted polycycloalkyl,
substituted or unsubstituted lower alkenyl,
substituted or unsubstituted cycloalkenyl,
substituted or unsubstituted lower alkoxy,
substituted or unsubstituted lower alkanoyloxy,
substituted or unsubstituted aralkyl,
substituted or unsubstituted heteroaralkyl,
substituted or unsubstituted aryl,
substituted or unsubstituted heteroaryl, or
a substituted or unsubstituted non-aromatic heterocyclic group;
R^{2a}, R^{2b}, R^{3a}, R^{3b}, R^{4a}, R^{4b}, R^{5a}, and R^{5b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl;
R⁶, R⁷, R⁸, and R⁹ are each, independently,
hydrogen,
hydroxy,
substituted or unsubstituted lower alkyl,
substituted or unsubstituted lower alkoxy,
substituted or unsubstituted lower alkanoyl,
substituted or unsubstituted lower alkanoyloxy,
substituted or unsubstituted lower alkoxycarbonyl,
substituted or unsubstituted aryl,
substituted or unsubstituted heteroaryl,
halogen,
—CN,
—NO₂,
—C(═O)—OR^{17a},
—NR^{17a}R^{17b}, or
—C(═O)—NR^{17a}R^{17b}, wherein
R^{17a} and R^{17b} are each, independently,
hydrogen,
substituted or unsubstituted lower alkyl,
substituted or unsubstituted cycloalkyl,
substituted or unsubstituted aryl, or
substituted or unsubstituted aralkyl, or
R^{17a} and R^{17b} taken together with the nitrogen atom to which they are bonded form a substituted or unsubstituted heterocyclic group containing at least one nitrogen atom;
R^{10a}, R^{10b}, R^{11a}, and R^{11b} are each, independently,
hydrogen,
substituted or unsubstituted lower alkyl,
substituted or unsubstituted cycloalkyl,
substituted or unsubstituted aryl,
substituted or unsubstituted aralkyl,
substituted or unsubstituted heteroaralkyl, or
substituted or unsubstituted lower alkoxyalkyl;
R^{12a} and R^{12b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl, or
R^{12a} and R^{12b} taken together with the nitrogen atom to which they are bonded form a substituted or unsubstituted heterocyclic group containing at least one nitrogen atom;
R^{13a} and R^{13b} are each, independently,
hydrogen,
substituted or unsubstituted lower alkyl,
substituted or unsubstituted cycloalkyl,
substituted or unsubstituted aryl,
substituted or unsubstituted aralkyl, or
substituted or unsubstituted heteroaralkyl,
wherein when p is 2 or more, multiple R^{13a}'s are independently the same or different and multiple R^{13b}'s are independently the same or different;
m is an integer from 0 to 4;
n is an integer from 0 to 6;
p is an integer from 0 to 9; and
q is an integer from 0 to 5

11. The method according to claim 10, wherein said disease is an autoimmune disease.

12. The method according to claim 10, wherein said disease is an allergic disease or condition.
